(19) United States
(12) Patent Application Publication
(10) Pub. No.: US 2022/0251595 A1
(43) Pub. Date: Aug. 11, 2022

(54) ENGINEERED ATRLP23 PATTERN RECOGNITION RECEPTORS AND METHODS OF USE

(71) Applicant: Two Blades Foundation, Evanston, IL (US)

(72) Inventors: Freddy France Guy Boutrot, Mirebeau (FR); Carolina Grandellis, Norwich (GB); Kamil Witek, Norwich (GB); Cyril B. Zipfel, Zurich (CH)

(73) Assignee: Two Blades Foundation, Evanston, IL (US)

(21) Appl. No.: 17/621,899

(22) PCT Filed: Jun. 26, 2020
(86) PCT No.: PCT/US2020/039730

§ 371 Date: Dec. 22, 2021

Related U.S. Application Data
(60) Provisional application No. 62/867,327, filed on Jun. 27, 2019.

(51) Int. Cl. C12N 15/82; C07K 14/415; C12N 9/12

(52) U.S. Cl. CPC C12N 15/8279; C07K 2319/00; C12N 9/1205; C07K 14/415

(57) ABSTRACT

Compositions and methods for enhancing the disease resistance of plants are provided. The invention provides compositions comprising engineered, pattern recognition receptors which comprise one or more domains derived from the receptor-like protein AtRLP23, particularly a leucine-rich repeat domain, and one or more other domains including, for example, a kinase domain from a receptor-like kinase. The compositions further comprise nucleic acid molecules encoding the engineered proteins and plants, plant cells, and other host cells comprising such nucleic acid molecules and/or the engineered proteins. The invention additionally provides methods for making and using the engineered proteins and nucleic acid molecules encoding the engineered proteins.

### CROSS-REFERENCE TO RELATED APPLICATION

This application claims the benefit of U.S. Provisional Patent Application No. 62/867,327, filed Jun. 27, 2019, which is hereby incorporated herein in its entirety by reference.

### REFERENCE TO A SEQUENCE LISTING SUBMITTED AS A TEXT FILE

The official copy of the sequence listing is submitted electronically via EFS-Web as an ASCII formatted sequence listing with a file named 070294-0169SEQLST.TXT, created on Jun. 23, 2020 and having a size of 963 kilobytes, and is filed concurrently with the specification. The sequence listing contained in this ASCII formatted document is part of the specification and is herein incorporated by reference in its entirety.

### FIELD OF THE INVENTION

The present invention relates to the fields of plant disease resistance and crop plant improvement, particularly to making engineered plant disease resistance proteins and using such engineered disease resistance proteins to enhance the resistance of crop plants to plant diseases.

### BACKGROUND OF THE INVENTION

According to the United Nations estimates, the world's human population is expected to increase by nearly 1 billion by 2030 (United Nations, Department of Economic and Social Affairs, Population Division (2017) “World Population Prospects: The 2017 Revision, Key Findings and Advance Tables,” Working Paper No. ESA/P/WP/248, p. 1). Due to the projected increase in world's human population and also the projected loss of land available for agricultural production, agricultural scientists need to increase agricultural productivity to keep pace with the growing demand for agricultural products for consumption by humans, livestock, aqua-cultured organisms, and pets. A variety of strategies will need to be employed to increase agricultural productivity, which may include improved crop plant cultivars and traits, new and improved agricultural chemicals, improved fertilizers and biologics, and improved crop production systems.

While synthetic agricultural chemicals will continue to be important for intensive crop production in developed countries, many farmers in developing countries cannot afford to use synthetic agricultural chemicals on their crops. Additionally, in developed nations, consumers are demanding sustainably produced food products. The sustainable intensification of agriculture will require increased use of genetic solutions instead of chemical solutions (e.g. synthetic pesticides) to protect crops against pathogens and pests (Jones et al. (2014) Philos. T Roy. Soc. B 369:20130087). Such genetic solutions include, for example, crop plants which have been bred to be resistant to pathogens through introgression of naturally occurring resistance (R) genes which provide the plant with resistance against plant pathogens such as, for example, bacteria, oomycetes, viruses, fungi, and nematodes.

While R genes have been successfully used to enhance the resistance of crop plants to plant pathogens, the resistance conferred to plants by most R genes has not been durable as pathogens evolve to overcome the resistance provided by the R genes. The reliance on monocultures in modern agriculture promotes the rapid emergence of new virulent isolates of plant pathogens, because plant pathogens experience a strong selective pressure as cultivars with new R genes are released (McDonald & Linde (2002) Euphytica 124:163-180). Thus, to stay ahead of the rapidly evolving pathogens, it is imperative for plant scientists not only to discover and integrate new R genes into crop plants, but also to develop new strategies for enhancing the resistance of crop plants. Such new strategies may involve plant proteins that have roles in the very early stages of the plant signal transduction pathway that is initiated following a pathogen attack such as the pattern recognition receptors (PRRs) that plants employ to detect pathogen-associated molecular patterns (PAMPs).

### BRIEF SUMMARY OF THE INVENTION

The present invention provides methods for making engineered AtRLP23 proteins. AtRLP23 is a receptor-like protein (RLP) that is a pattern recognition receptor (PRR) capable of recognizing, in a plant, pathogen-associated molecular patterns (PAMPs) derived from the necrosis- and ethylene-inducing protein 1 (Nep1)-like protein family of PAMPs that are known to occur in bacteria, fungi, and oomycetes. The engineered AtRLP23 proteins of the present invention are synthetic or artificial (i.e. non-naturally occurring) proteins. In some embodiments, the methods for making engineered AtRLP23 proteins involve producing chimeric proteins that have the receptor function of AtRLP23 and the kinase domain derived from a receptor-like kinase (RLK). In particular, the methods comprise producing a polypeptide comprising an amino acid sequence having the following domains in operable linkage and in an N-terminal-to-C-terminal direction: the leucine-rich-repeat (LRR) domain from AtRLP23 or a derivative thereof that is capable of recognizing in a plant, a pathogen-associated molecular pattern derived from a Nep1-like protein (NLP); an extra-juxtamembrane (eJM) domain, a transmembrane (TM) domain, and a kinase domain derived from an RLK. If desired, the polypeptide can further comprise a signal peptide (SP) domain that is operably linked to the N-terminal end of LRR domain. The SP, eJM, and TM domains can be derived from AtRLP23 or one or more other PRRs, particularly one or more other RLPs.

The present invention further provides methods for making recombinant nucleic acid molecules that encode the engineered AtRLP23 proteins of the present invention. In some embodiments of the invention, such methods involve synthesizing in vitro nucleic acid molecules that encode the engineered AtRLP23 proteins of the present invention. In other embodiments, the methods involve genome editing to create in plant cells genes nucleotide sequences that encode the engineered AtRLP23 proteins of the present invention. In still other embodiments, the methods involve both in vitro synthesis of at least a portion of the nucleic acid molecule or sequence encoding an engineered AtRLP23 protein and genome editing.

The present invention further provides of methods for producing plants with enhanced resistance to plant pathogens comprising modifying a plant cell to express an engineered AtRLP23 protein of the present invention. In some embodiments, the methods comprise modifying a plant cell by introducing into a plant cell a nucleic acid molecule or sequence comprising a nucleotide sequence that encodes an engineered AtRLP23 protein, and optionally regenerating the plant cell into a plant comprising the nucleic acid molecule or sequence stably incorporated in its genome, wherein the regenerated plant comprises enhanced resistance to one or more plant pathogens, particularly plant pathogens that comprise an NLP, more particularly bacterial, fungal, and oomycete pathogens that comprise an NLP. In other embodiments, the methods comprise modifying a plant cell by modifying the genome of a plant or at least one cell thereof to comprise a polynucleotide comprising a nucleotide sequence that encodes an engineered AtRLP23 protein using genome editing methods to cause single-strand or double-strand breaks in specific locations in the genomes of cells, whereby a plant comprising the polynucleotide is produced and comprises enhanced resistance to one or more plant pathogens, particularly plant pathogens that comprise an NLP, more particularly bacterial, fungal, and oomycete pathogens that comprise an NLP.

Methods of using the plants of the present invention in agricultural crop production to limit plant diseases caused by plant pathogens are also provided. In some embodiments, the methods comprise planting a seed, seedling, a tuber, or other plant part, wherein the seed, seedling, a tuber, or other plant part comprises a nucleic acid molecule comprising a nucleotide sequence encoding an engineered AtRLP23 of the present invention. In some other embodiments, the methods comprise planting a seed, seedling, a tuber, or other plant part, wherein the seed, seedling, a tuber, or other plant part from a plant that has been modified to be capable of expression of at least one engineered AtRLP23 of the present invention. The methods further comprise growing the plant under environmental conditions that are favorable for the growth and development of the plant, and then optionally harvesting from the plant at least one seed, tuber, fruit, flower, or other plant part or parts.

Additionally provided are the engineered AtRLP23 proteins and nucleic acid molecules encoding the engineered AtRLP23 proteins of the present invention. Further provided are plants, plant parts, seeds, fruits, tubers, plant cells, other host cells, expression cassettes, and vectors comprising one or more of the nucleic acid molecules of the present invention.

### BRIEF DESCRIPTION OF THE FIGURES

FIG. 1 is a graphical representation of the protein domain structure of the receptor-like proteins (RLPs) AtRLP23 (SEQ ID NO: 2) and AtRLP42 (SEQ ID NO: 4), of the receptor-like kinases (RLKs) AtEFR (SEQ ID NO: 6) and AtSOBIR1 (SEQ ID NO: 8), and of the chimeric receptor AtRLP23-eJMAtRLP42-AtEFR-3×FLAG (SEQ ID NO: 62). These receptors typically contain a signal peptide (SP), an apoplastic ectodomain rich in Leucine-Rich Repeats (LRR domain), an apoplastic extra-juxtamembrane (eJM) domain, a transmembrane (TM) domain, and either a cytoplasmic carboxy-terminal extension (tail) or a kinase domain for the RLPs or RLKs, respectively.

FIG. 2 is a graphical representation of the calcium burst measured in corn protoplasts transiently transfected with 10 μg of ZmUbi::R-GECO1.2::rbcS (SEQ ID NO: 75) and 10 μg of either pUC19 (SEQ ID NO: 74) or the 2×35S+Ω promoter construct (SEQ ID NO: 72) operably linked to AtRLP23-3×FLAG coding sequence (SEQ ID NO: 13) which is operably linked to rbcS terminator (SEQ ID NO: 71). Fluorescence was measured in response to 1 μM PpNLP20 (AIMYSWYFPKDSPVTGLGHR, SEQ ID NO: 63); prepared by diluting a 100 μM stock solution with the protoplast incubation buffer). Each measurement was performed in 384-well multiplate using a 25 μL aliquot from a 120 μL solution containing 0.32×10⁶ corn cells transiently transfected. 12.5 μL of 3 μM PpNLP20 were then added before immediately monitoring the fluorescence of the samples with excitation 556 nm and emission 585 nm. Values are average and standard error (n=8).

FIG. 3 is a graphical representation of the calcium burst measured in corn protoplasts transiently transfected with 10 μg of ZmUbi::R-GECO1.2::rbcS (SEQ ID NO: 75) and 10 μg of either the AtRLP23-3×FLAG (SEQ ID NO: 13), AtRLP23-eJM(EEEE/ADQ−)-3×FLAG (SEQ ID NO: 45), AtRLP23-eJMAtRLP1-3×FLAG (SEQ ID NO: 49), AtRLP23-eJMAtRLP42-3×FLAG (SEQ ID NO: 57) or AtRLP23-eJMVe1-3×FLAG (SEQ ID NO: 53), all of which were operably linked to 2×35S+Ω promoter construct and the rbcS terminator. Fluorescence was measured in response to the protoplast incubation buffer or to 1 μM PpNLP20 (SEQ ID NO: 63); prepared by diluting a 100 μM stock solution with the protoplast incubation buffer). Each measurement was performed in 96-well multiplate using a 100 μL aliquot from a 120 μL solution containing 0.32×10⁶ corn cells transiently transfected. 50 μL of either buffer or 3 μM PpNLP20 were then added before immediately monitoring the fluorescence of the samples with excitation 556 nm and emission 585 nm. For each fluorescence measurement, the transient increase of fluorescence was measured over background determined as the average signal in presence of buffer. Total transient increase of fluorescence was measured for 40 minutes from the time of treatment. Values are average and standard error (n=8). Statistical significance by comparison with the total transient increase of fluorescence observed with AtRLP23-3×FLAG+1 μM PpNLP20 using one-way ANOVA test followed by Dunnett's post-test, n.s. non-significant, ***P<0.001.

FIG. 4 is a graphical representation of the calcium burst measured in corn protoplasts transiently transfected with 10 μg of ZmUbi::R-GECO1.2::rbcS (SEQ ID NO: 75) and 10 μg of either pUC19 (SEQ ID NO: 74) or the following AtRLP23 constructs: AtRLP23-3×FLAG (SEQ ID NO: 13), AtRLP23-OsXA21-3×FLAG (SEQ ID NO: 21), AtRLP23-AtEFR-3×FLAG (SEQ ID NO: 17), AtRLP23+TM-AtEFR-3×FLAG (SEQ ID NO: 33), AtRLP23-AtBAK1-3×FLAG (SEQ ID NO: 25), AtRLP23+TM-AtBAK1-3×FLAG (SEQ ID NO: 37), AtRLP23-AtSOBIR1-3×FLAG (SEQ ID NO: 29) or AtRLP23+TM-AtSOBIR1-3×FLAG (SEQ ID NO: 41). All of the AtRLP23 constructs were expressed under the control of an operably linked 2×35S+Ω promoter construct (SEQ ID NO: 72) and also contained an operably linked rbcS terminator (SEQ ID NO: 73). Fluorescence was measured in response to the protoplast incubation buffer or to 1 μM PpNLP20 (SEQ ID NO: 63); prepared by diluting a 100 μM stock solution with the protoplast incubation buffer). Each measurement was performed in 384-well multiplate using a 25 μL aliquot from a 120 μL solution containing 0.32×10⁶ corn cells transiently transfected. 12.5 μL of either buffer or 3 μM PpNLP20 were then added before immediately monitoring the fluorescence of the samples with excitation 556 nm and emission 585 nm. For each fluorescence measurement, the transient increase of fluorescence was measured over background determined as the average signal in presence of buffer. Total transient increase of fluorescence was measured for 40 minutes from the time of treatment. Values are average (n=8).

FIG. 5 is a graphical representation of the calcium burst measured in corn protoplasts transiently transfected with 10 μg of ZmUbi::Apoaequorin::rbcS (SEQ ID NO: 76) and 10 μg of a construct comprising AtRLP23-eJMAtRLP42-AtEFR-3×FLAG (SEQ ID NO: 61) expressed under the control of an operably linked 2×35S+Ω promoter construct (SEQ ID NO: 72) and also containing an operably linked rbcS terminator (SEQ ID NO: 73). Luminescence was measured in response to the protoplast incubation buffer or to 1 μM PpNLP20 (AIMYSWYFPKDSPVTGLGHR, SEQ ID NO: 63; prepared by diluting a 100 μM stock solution with the protoplast incubation buffer). Each measurement was performed in 96-well multiplate using a 100 μL aliquot from a 120 μL solution containing 0.32×10⁶ corn cells transiently transfected and incubated for 2 hours with 1 μM coelenterazine. 50 μL of 3 μM PpNLP20 were then added before immediately monitoring the luminescence of the samples. Values are average and standard error (n=8).

FIG. 6 is a graphical representation of the calcium burst measured in corn protoplasts transiently transfected with 10 μg of ZmUbi::Apoaequorin::rbcS (SEQ ID NO: 76) and 10 of one of the following constructs: AtRLP23-eJMAtRLP42-3×FLAG (SEQ ID NO: 57) or AtRLP23-eJMAtRLP42-AtEFR-3×FLAG (SEQ ID NO: 61). Both constructs were expressed under the control of an operably linked 2×35S+Ω promoter construct (SEQ ID NO: 72) and also contained an operably linked rbcS terminator (SEQ ID NO: 73). Luminescence was measured in response to the protoplast incubation buffer or to 1 μM PpNLP20 (SEQ ID NO: 63), CgNLP24b (SEQ ID NO: 64), FgNLP24c (SEQ ID NO: 65), FvNLP24a (SEQ ID NO: 66) or SmNLP24 (SEQ ID NO: 67). Each measurement was performed in 96-well multiplate using a 100 μL aliquot from a 120 μL solution containing 0.32×10⁶ corn cells transiently transfected and incubated for 2 hours with 1 μM coelenterazine. 50 μL of buffer or 3 μM of either PpNLP20, CgNLP24b, FgNLP24c, FvNLP24a or SmNLP24 solutions are then added before immediately monitoring the luminescence of the samples. Total transient increase of luminescence was measured for 40 minutes from the time of treatment. Values are average and standard error (n=8). Statistical significance by comparison with the corresponding buffer treatments using one-way ANOVA test followed by Dunnett's post-test, *P<0.05, **P<0.01, ***P<0.001.

FIG. 7 is a graphical representation of the calcium burst measured in corn protoplasts transiently transfected with 10 μg of ZmUbi::Apoaequorin::rbcS (SEQ ID NO: 76) and 10 of AtRLP23-eJMAtRLP42-AtEFR-3×FLAG (SEQ ID NO: 61) expressed under the control of an operably linked 2×35S+Ω promoter construct (SEQ ID NO: 72) and also containing an operably linked rbcS terminator (SEQ ID NO: 73). Luminescence was measured in response to the protoplast incubation buffer or to 1 μM PpNLP20 (SEQ ID NO: 63), SmNLP24 (SEQ ID NO: 67), AfNLP24a (SEQ ID NO: 68), ApNLP24a (SEQ ID NO: 71), AfNLP24b (SEQ ID NO: 69) or AfNLP24c (SEQ ID NO: 70). Each measurement was performed in 96-well multiplate using a 100 μL aliquot from a 120 μL solution containing 0.32×10⁶ corn cells transiently transfected and incubated for 2 hours with 1 μM coelenterazine. 50 μL of buffer or 3 μM of either PpNLP20, SmNLP24, AfNLP24a, ApNLP24a, AfNLP24b or AfNLP24c solutions are then added before immediately monitoring the luminescence of the samples. Total transient increase of luminescence was measured for 40 minutes from the time of treatment. Values are average and standard error (n=8). Statistical significance by comparison with the corresponding buffer treatments using one-way ANOVA test followed by Dunnett's post-test, ***P<0.001.

FIG. 8 is a graphical representation of the calcium burst measured in corn protoplasts transiently transfected with 10 μg of one of the following constructs: AtRLP23-eJMAtRLP42-AtEFR-3×FLAG (SEQ ID NO: 61) or AtRLP23-AtPEPR1-3×FLAG (SEQ ID NO: 572). Both constructs were expressed under the control of an operably linked 2×35S+Ω promoter construct (SEQ ID NO: 72) and an operably linked rbcS terminator (SEQ ID NO: 73). Luminescence (RLU) was measured in response to the protoplast incubation buffer or to SmNLP24 (SEQ ID NO: 67), CgNLP24b (SEQ ID NO: 64), FgNLP24c (SEQ ID NO: 65), or FvNLP24a (SEQ ID NO: 66). Each measurement was performed in 96-well multiplate using a 100 μL aliquot from a 120 μL solution containing 0.32×10⁶ corn protoplasts transiently transfected and incubated for 2 hours with 1 μM coelenterazine. 50 μL of buffer or 3 μM of either SmNLP24, CgNLP24b, FgNLP24c or FvNLP24a solutions were then added before immediately monitoring the luminescence of the samples. Total transient increase of luminescence (RLU) was measured for 40 minutes from the time of treatment. Values are average and standard error (n=4). Statistical significance between buffer and peptide treatments was tested using ANOVA post-hoc pairwise comparisons performed with Bonferroni. All pairs showed statistically significant difference of p<0.001, except for AtRLP2-AtPEPR1-3×FLAG treated with FgNLP24a, where p-value <0.001.

FIG. 9 is a graphical representation is the results of a Diplodia stalk rot assay using greenhouse-grown, transgenic corn plants expressing an AtRLP23-eJMAtRLP42-AtEFR construct which comprises the ectodomain of AtRLP23, the eJM domain of AtRLP42, the TM and cytoplasmic domains of AtEFR. “Events 1-10” are ten individual transgenic events produced from the transformation of corn with the construct. “Transformation germplasm” represents untransformed, control plants. The errors bars indicate standard error of the difference.

FIG. 10 is a graphical representation is the results of a Diplodia stalk rot assay using greenhouse-grown, transgenic corn plants expressing an AtRLP23-eJMAtRLP42-AtRLP23-TM+C-term construct which comprises the ectodomain of AtRLP23, the eJM domain of AtRLP42, and the TM and the cytoplasmic domain of AtRLP23. “Events 1-9” are nine individual transgenic events produced from the transformation of corn with the construct. “Transformation germplasm” represents untransformed, control plants. The errors bars indicate standard error of the difference.

FIG. 11 is a graphical representation is the results of a Diplodia stalk rot assay using greenhouse-grown, transgenic corn plants expressing an AtRLP23-eJMAtRLP42-TMAtRLP23-AtSOBIR1 construct which comprises the ectodomain of AtRLP23, the eJM domain of AtRLP42, the TM domain of AtRLP23, and the cytoplasmic domain of AtSOBIR1. “Events 1-9” are nine individual transgenic events produced from the transformation of corn with the construct. “Transformation germplasm” represents untransformed, control plants. The errors bars indicate standard error of the difference.

### SEQUENCE LISTING

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth the nucleotide sequence of the coding region of AtRLP23 from Arabidopsis thaliana. If desired, a stop codon (e.g. TAA, TAG, or TGA) can be operably linked to the 3′ end of a nucleic acid molecule comprising SEQ ID NO: 1. It is noted that the native stop codon of AtRLP23 is TAG.

SEQ ID NO: 2 sets forth the amino acid sequence of AtRLP23, the protein encoded by AtRLP23.

SEQ ID NO: 3 sets forth the nucleotide sequence of the coding region of AtRLP42 from Arabidopsis thaliana. If desired, a stop codon (e.g. TAA, TAG, or TGA) can be operably linked to the 3′ end of a nucleic acid molecule comprising SEQ ID NO: 3. It is noted that the native stop codon of AtRLP23 is TAA.

SEQ ID NO: 4 sets forth the amino acid sequence of AtRLP42, the protein encoded by AtRLP42.

SEQ ID NO: 5 sets forth the nucleotide sequence of the coding region of AtEFR from Arabidopsis thaliana. If desired, a stop codon (e.g. TAA, TAG, or TGA) can be operably linked to the 3′ end of a nucleic acid molecule comprising SEQ ID NO: 5. It is noted that the native stop codon of AtEFR is TAG.

SEQ ID NO: 6 sets forth the amino acid sequence of AtEFR, the protein encoded by AtEFR.

SEQ ID NO: 7 sets forth the nucleotide sequence of the coding region of AtSOBIR1 from Arabidopsis thaliana. If desired, a stop codon (e.g. TAA, TAG, or TGA) can be operably linked to the 3′ end of a nucleic acid molecule comprising SEQ ID NO: 7. It is noted that the native stop codon of AtSOBIR1 is TAG.

SEQ ID NO: 8 sets forth the amino acid sequence of AtSOBIR1, the protein encoded by AtSOBIR1.

SEQ ID NO: 9 sets forth the nucleotide sequence of the coding region of SlVe1 from Solanum lycopersicum. If desired, a stop codon (e.g. TAA, TAG, or TGA) can be operably linked to the 3′ end of a nucleic acid molecule comprising SEQ ID NO: 9. It is noted that the native stop codon of SlVe1 is TGA.

SEQ ID NO: 10 sets forth the amino acid sequence of SlVe1, the protein encoded by SlVe1.

SEQ ID NO: 11 sets forth the nucleotide sequence of the coding region of OsXA21 from Oryza sativa. If desired, a stop codon (e.g. TAA, TAG, or TGA) can be operably linked to the 3′ end of a nucleic acid molecule comprising SEQ ID NO: 11. It is noted that the native stop codon of OsXA21 is TGA.

SEQ ID NO: 12 sets forth the amino acid sequence of OsXA21, the protein encoded by OsXA21.

SEQ ID NO: 13 sets forth the nucleotide sequence of the AtRLP23-3×FLAG polynucleotide construct. The construct comprises a first nucleotide sequence encoding AtRLP23 (nucleotides 1-2670) which includes the apoplastic domain, the extra-juxtamembrane (eJM) domain, the transmembrane (TM) domain and the cytoplasmic domain, operably linked a second nucleotide sequence encoding a linking amino acid (nucleotides 2671-2673) which is operably linked to a nucleotide sequence encoding the 3×FLAG peptide (nucleotides 2674-2754). If desired, a stop codon (e.g. TAA, TAG, or TGA) can be operably linked to the 3′ end of a nucleic acid molecule comprising SEQ ID NO: 13. It is noted that in the Examples below, the stop codon TGA was used with this construct.

SEQ ID NO: 14 sets forth the amino acid sequence of AtRLP23-3×FLAG protein encoded by SEQ ID NO: 13. The AtRLP23-3×FLAG protein comprises AtRLP23 (amino acids 1-890) which includes the apoplastic, eJM, TM, cytoplasmic domains operably linked to a linking amino acid (amino acid 891) which is operably linked to the 3×FLAG peptide (amino acids 892-918).

SEQ ID NO: 15 sets forth the nucleotide sequence of the AtRLP23-AtEFR polynucleotide construct. The construct comprises a first nucleotide sequence encoding the apoplastic and eJM domains of AtRLP23 (nucleotides 1-2550) operably linked to a second nucleotide sequence comprising a coding sequence for the AtEFR TM domain and the first of two parts of the AtEFR cytoplasmic domain (nucleotides 2551-3304), an AtEFR intron (nucleotides 3305-3391), and the coding sequence of the second part of AtEFR cytoplasmic domain (nucleotides 3392-3783). If desired, a stop codon (e.g. TAA, TAG, or TGA) can be operably linked to the 3′ end of a nucleic acid molecule comprising SEQ ID NO: 15.

SEQ ID NO: 16 sets forth the amino acid sequence of AtRLP23-AtEFR protein encoded by SEQ ID NO: 15. The AtRLP23-AtEFR protein comprises a first polypeptide comprising the AtRLP23 apoplastic and eJM domains (amino acids 1-850) operably linked to a second polypeptide comprising the AtEFR TM and cytoplasmic domains (amino acids 851-1232).

SEQ ID NO: 17 sets forth the nucleotide sequence of the AtRLP23-AtEFR-3×FLAG polynucleotide construct. The construct comprises a first nucleotide sequence encoding the apoplastic and eJM domains of AtRLP23 (nucleotides 1-2550), operably linked to a second nucleotide sequence comprising a coding sequence for the AtEFR TM domain and the first of two parts of the AtEFR cytoplasmic domain (nucleotides 2551-3304), an AtEFR intron (nucleotides 3305-3391), and the coding sequence of the second part of AtEFR cytoplasmic domain (nucleotides 3392-3783), operably linked to third nucleotide sequence encoding a linker (nucleotides 3784-3789), operably linked to a fourth nucleotide sequence encoding the 3×FLAG peptide (nucleotides 3790-3870). If desired, a stop codon (e.g. TAA, TAG, or TGA) can be operably linked to the 3′ end of a nucleic acid molecule comprising SEQ ID NO: 17. It is noted that in the Examples below, the stop codon TGA was used with this construct.

SEQ ID NO: 18 sets forth the amino acid sequence of AtRLP23-AtEFR-3×FLAG protein encoded by SEQ ID NO: 17. The AtRLP23-AtEFR-3×FLAG protein comprises a first polypeptide comprising the AtRLP23 apoplastic and eJM domains (amino acids 1-850), operably linked to a second polypeptide comprising the AtEFR TM and cytoplasmic domains (amino acids 851-1232), operably linked to a linker dipeptide (amino acids 1233-1234), operably linked to the 3×FLAG peptide (amino acids 1235-1261).

SEQ ID NO: 19 sets forth the nucleotide sequence of the AtRLP23-OsXA21 polynucleotide construct. The construct comprises a first nucleotide sequence encoding the apoplastic and eJM domains of AtRLP23 (nucleotides 1-2550) operably linked to a second nucleotide sequence encoding the OsXA21 TM and cytoplasmic domains (nucleotides 2551-3675). If desired, a stop codon (e.g. TAA, TAG, or TGA) can be operably linked to the 3′ end of a nucleic acid molecule comprising SEQ ID NO: 19.

SEQ ID NO: 20 sets forth the amino acid sequence of AtRLP23-OsXA21 protein encoded by SEQ ID NO: 19. The AtRLP23-OsXA21 protein comprises a first polypeptide comprising the AtRLP23 apoplastic and eJM domains (amino acids 1-850) operably linked to a second polypeptide comprising the OsXA21 TM and cytoplasmic domains (amino acids 851-1225).

SEQ ID NO: 21 sets forth the nucleotide sequence of the AtRLP23-OsXA21-3×FLAG polynucleotide construct. The construct comprises a first nucleotide sequence encoding the apoplastic and eJM domains of AtRLP23 (nucleotides 1-2550), operably linked to a second nucleotide sequence encoding the OsXA21 TM and cytoplasmic domains (nucleotides 2551-3675), operably linked to third nucleotide sequence encoding a linking amino acid (nucleotides 3676-3678), operably linked to a fourth nucleotide sequence encoding the 3×FLAG peptide (nucleotides 3679-3762). If desired, a stop codon (e.g. TAA, TAG, or TGA) can be operably linked to the 3′ end of a nucleic acid molecule comprising SEQ ID NO: 21. It is noted that in the Examples below, the stop codon TGA was used with this construct.

SEQ ID NO: 22 sets forth the amino acid sequence of AtRLP23-OsXA21-3×FLAG protein encoded by SEQ ID NO: 21. The AtRLP23-OsXA21-3×FLAG protein comprises a first polypeptide comprising the AtRLP23 apoplastic and eJM domains (amino acids 1-850), operably linked to a second polypeptide comprising the OsXA21 TM and cytoplasmic domains (amino acids 851-1225), operably linked to a linking amino acid (amino acid 1226), operably linked to the 3×FLAG peptide (amino acids 1227-1253).

SEQ ID NO: 23 sets forth the nucleotide sequence of the AtRLP23-AtBAK1 polynucleotide construct. The construct comprises a first nucleotide sequence encoding the apoplastic and eJM domains of AtRLP23 (nucleotides 1-2550) operably linked to a second nucleotide sequence comprising a coding sequence for the AtBAK1 TM domain and the first of four parts of the AtBAK1 cytoplasmic domain (nucleotides 2551-2671), a first AtBAK1 intron (nucleotides 2672-2748), the coding sequence of the second part of AtBAK1 cytoplasmic domain (nucleotides 2749-3090), a second AtBAK1 intron (nucleotides 3091-3187), the coding sequence of the third part of AtBAK1 cytoplasmic domain (nucleotides 3188-3582), a third AtBAK1 intron (nucleotides 3583-3663), the coding sequence of the fourth part of AtBAK1 cytoplasmic domain (nucleotides 3664-3984). If desired, a stop codon (e.g. TAA, TAG, or TGA) can be operably linked to the 3′ end of a nucleic acid molecule comprising SEQ ID NO: 23.

SEQ ID NO: 24 sets forth the amino acid sequence of AtRLP23-AtBAK1 protein encoded by SEQ ID NO: 23. The AtRLP23-AtBAK1 protein comprises a first polypeptide comprising the AtRLP23 apoplastic and eJM domains (amino acids 1-850) operably linked to a second polypeptide comprising the AtBAK1 TM and cytoplasmic domains (amino acids 851-1242).

SEQ ID NO: 25 sets forth the nucleotide sequence of the AtRLP23-AtBAK1-3×FLAG polynucleotide construct. The construct comprises a first nucleotide sequence encoding the apoplastic and eJM domains of AtRLP23 (nucleotides 1-2550) operably linked to a second nucleotide sequence comprising a coding sequence for the AtBAK1 TM domain and the first of four parts of the AtBAK1 cytoplasmic domain (nucleotides 2551-2671), a first AtBAK1 intron (nucleotides 2672-2748), the coding sequence of the second part of AtBAK1 cytoplasmic domain (nucleotides 2749-3090), a second AtBAK1 intron (nucleotides 3091-3187), the coding sequence of the third part of AtBAK1 cytoplasmic domain (nucleotides 3188-3582), a third AtBAK1 intron (nucleotides 3583-3663), the coding sequence of the fourth part of AtBAK1 cytoplasmic domain (nucleotides 3664-3984), operably linked to third nucleotide sequence encoding a linking amino acid (nucleotides 3985-3987), operably linked to a fourth nucleotide sequence encoding the 3×FLAG peptide (nucleotides 3988-4068). If desired, a stop codon (e.g. TAA, TAG, or TGA) can be operably linked to the 3′ end of a nucleic acid molecule comprising SEQ ID NO: 25. It is noted that in the Examples below, the stop codon TGA was used with this construct.

SEQ ID NO: 26 sets forth the amino acid sequence AtRLP23-AtBAK1-3×FLAG protein encoded by SEQ ID NO: 25. The AtRLP23-AtBAK1-3×FLAG protein comprises a first polypeptide comprising the AtRLP23 apoplastic and eJM domains (amino acids 1-850), operably linked to a second polypeptide comprising the AtBAK1 TM and cytoplasmic domains (amino acids 851-1243), operably linked to a linking amino acid (amino acid 1244), operably linked to the 3×FLAG peptide (amino acids 1245-1271).

SEQ ID NO: 27 sets forth the nucleotide sequence of the AtRLP23-AtSOBIR1 polynucleotide construct. The construct comprises a first nucleotide sequence encoding the apoplastic and eJM domains of AtRLP23 (nucleotides 1-2550) operably linked to a second nucleotide sequence encoding the AtSOBIR1 TM and cytoplasmic domains (nucleotides 2551-3624). If desired, a stop codon (e.g. TAA, TAG, or TGA) can be operably linked to the 3′ end of a nucleic acid molecule comprising SEQ ID NO: 27.

SEQ ID NO: 28 sets forth the amino acid sequence of AtRLP23-AtSOBIR1 protein encoded by SEQ ID NO: 27. The AtRLP23-AtSOBIR1 protein comprises a first polypeptide comprising the AtRLP23 apoplastic and eJM domains (amino acids 1-850) operably linked to a second polypeptide comprising the AtSOBIR1 TM and cytoplasmic domains (amino acids 851-1208).

SEQ ID NO: 29 sets forth the nucleotide sequence of the AtRLP23-AtSOBIR1-3×FLAG polynucleotide construct. The construct comprises a first nucleotide sequence encoding the apoplastic and eJM domains of AtRLP23 (nucleotides 1-2550), operably linked to a second nucleotide sequence encoding the AtSOBIR1 TM and cytoplasmic domains (nucleotides 2551-3624), operably linked to third nucleotide sequence encoding a linking amino acid (nucleotides 3625-3627), operably linked to a fourth nucleotide sequence encoding the 3×FLAG peptide (nucleotides 3628-3708). If desired, a stop codon (e.g. TAA, TAG, or TGA) can be operably linked to the 3′ end of a nucleic acid molecule comprising SEQ ID NO: 29. It is noted that in the Examples below, the stop codon TGA was used with this construct.

SEQ ID NO: 30 sets forth the amino acid sequence of AtRLP23-AtSOBIR1-3×FLAG protein encoded by SEQ ID NO: 29. The AtRLP23-AtSOBIR1-3×FLAG protein comprises a first polypeptide comprising the AtRLP23 apoplastic and eJM domains (amino acids 1-850), operably linked to a second polypeptide comprising the AtSOBIR1 TM and cytoplasmic domains (amino acids 851-1208), operably linked to a linking amino acid (amino acid 1209), operably linked to the 3×FLAG peptide (amino acids 1210-1236).

SEQ ID NO: 31 sets forth the nucleotide sequence of the AtRLP23+TM-AtEFR polynucleotide construct. The construct comprises a first nucleotide sequence encoding the apoplastic, eJM, and TM domains of AtRLP23 (nucleotides 1-2619) operably linked to a second nucleotide sequence comprising a coding sequence for first of two parts of the AtEFR cytoplasmic domain (nucleotides 2620-3298), an AtEFR intron (nucleotides 3299-3385), and the coding sequence of the second part of AtEFR cytoplasmic domain (nucleotides 3386-3777). If desired, a stop codon (e.g. TAA, TAG, or TGA) can be operably linked to the 3′ end of a nucleic acid molecule comprising SEQ ID NO: 31.

SEQ ID NO: 32 sets forth the amino acid sequence of AtRLP23+TM-AtEFR protein encoded by SEQ ID NO: 31. The AtRLP23+TM-AtEFR protein comprises a first polypeptide comprising the AtRLP23 apoplastic, eJM and TM domains (amino acids 1-873) operably linked to a second polypeptide comprising the AtEFR cytoplasmic domain (amino acids 874-1230).

SEQ ID NO: 33 sets forth the nucleotide sequence of the AtRLP23+TM-AtEFR-3×FLAG polynucleotide construct. The construct comprises a first nucleotide sequence encoding the apoplastic, eJM, and TM domains of AtRLP23 (nucleotides 1-2619), operably linked to a second nucleotide sequence comprising a coding sequence for first of two parts of the AtEFR cytoplasmic domain (nucleotides 2620-3298), an AtEFR intron (nucleotides 3299-3385), and the coding sequence of the second part of AtEFR cytoplasmic domain (nucleotides 3386-3777), operably linked to third nucleotide sequence encoding a linker (nucleotides 3778-3783), operably linked to a fourth nucleotide sequence encoding the 3×FLAG peptide (nucleotides 3784-3864). If desired, a stop codon (e.g. TAA, TAG, or TGA) can be operably linked to the 3′ end of a nucleic acid molecule comprising SEQ ID NO: 33. It is noted that in the Examples below, the stop codon TGA was used with this construct.

SEQ ID NO: 34 sets forth the amino acid sequence of AtRLP23+TM-AtEFR-3×FLAG protein encoded by SEQ ID NO: 33. The AtRLP23+TM-AtEFR-3×FLAG protein comprises a first polypeptide comprising the AtRLP23 apoplastic, eJM, and TM domains (amino acids 1-873), operably linked to a second polypeptide comprising the AtEFR cytoplasmic domain (amino acids 874-1230), operably linked to a linker dipeptide (amino acids 1231-1232), operably linked to the 3×FLAG peptide (amino acids 1233-1259).

SEQ ID NO: 35 sets forth the nucleotide sequence of the AtRLP23+TM-AtBAK1 polynucleotide construct. The construct comprises a first nucleotide sequence encoding the apoplastic, eJM, and TM domains of AtRLP23 (nucleotides 1-2619) operably linked to a second nucleotide sequence comprising a coding sequence for the first of four parts of the AtBAK1 cytoplasmic domain (nucleotides 2620-2659), a first AtBAK1 intron (nucleotides 2660-2736), the coding sequence of the second part of AtBAK1 cytoplasmic domain (nucleotides 2737-3078), a second AtBAK1 intron (nucleotides 3079-3175), the coding sequence of the third part of AtBAK1 cytoplasmic domain (nucleotides 3176-3570), a third AtBAK1 intron (nucleotides 3571-3651), the coding sequence of the fourth part of AtBAK1 cytoplasmic domain (nucleotides 3652-3972). If desired, a stop codon (e.g. TAA, TAG, or TGA) can be operably linked to the 3′ end of a nucleic acid molecule comprising SEQ ID NO: 35.

SEQ ID NO: 36 sets forth the amino acid sequence of AtRLP23+TM-AtBAK1 protein encoded by SEQ ID NO: 35. The AtRLP23+TM-AtBAK1 protein comprises a first polypeptide comprising the AtRLP23 apoplastic, eJM, and TM domains (amino acids 1-873) operably linked to a second polypeptide comprising the AtBAK1 cytoplasmic domain (amino acids 874-1239).

SEQ ID NO: 37 sets forth the nucleotide sequence of the AtRLP23+TM-AtBAK1-3×FLAG polynucleotide construct. The construct comprises a first nucleotide sequence encoding the apoplastic, eJM, and TM domains of AtRLP23 (nucleotides 1-2619) operably linked to a second nucleotide sequence comprising a coding sequence for the first of four parts of the AtBAK1 cytoplasmic domain (nucleotides 2620-2659), a first AtBAK1 intron (nucleotides 2660-2736), the coding sequence of the second part of AtBAK1 cytoplasmic domain (nucleotides 2737-3078), a second AtBAK1 intron (nucleotides 3079-3175), the coding sequence of the third part of AtBAK1 cytoplasmic domain (nucleotides 3176-3570), a third AtBAK1 intron (nucleotides 3571-3651), the coding sequence of the fourth part of AtBAK1 cytoplasmic domain (nucleotides 3652-3972), operably linked to third nucleotide sequence encoding a linking amino acid (nucleotides 3973-3975), operably linked to a fourth nucleotide sequence encoding the 3×FLAG peptide (nucleotides 3976-4056). If desired, a stop codon (e.g. TAA, TAG, or TGA) can be operably linked to the 3′ end of a nucleic acid molecule comprising SEQ ID NO: 37. It is noted that in the Examples below, the stop codon TGA was used with this construct.

SEQ ID NO: 38 sets forth the amino acid sequence AtRLP23+TM-AtBAK1-3×FLAG protein encoded by SEQ ID NO: 37. The AtRLP23+TM-AtBAK1-3×FLAG protein comprises a first polypeptide comprising the AtRLP23 apoplastic, eJM, and TM domains (amino acids 1-873), operably linked to a second polypeptide comprising the AtBAK1 cytoplasmic domain (amino acids 874-1239), operably linked to a linking amino acid (amino acid 1240), operably linked to the 3×FLAG peptide (amino acids 1241-1267).

SEQ ID NO: 39 sets forth the nucleotide sequence of the AtRLP23+TM-AtSOBIR1 polynucleotide construct. The construct comprises a first nucleotide sequence encoding the apoplastic, eJM, and TM domains of AtRLP23 (nucleotides 1-2619) operably linked to a second nucleotide sequence encoding the AtSOBIR1 cytoplasmic domain (nucleotides 2620-3615). If desired, a stop codon (e.g. TAA, TAG, or TGA) can be operably linked to the 3′ end of a nucleic acid molecule comprising SEQ ID NO: 39.

SEQ ID NO: 40 sets forth the amino acid sequence of AtRLP23+TM-AtSOBIR1 protein encoded by SEQ ID NO: 39. The AtRLP23+TM-AtSOBIR1 protein comprises a first polypeptide comprising the AtRLP23 apoplastic, eJM, and TM domains (amino acids 1-873) operably linked to a second polypeptide comprising the AtSOBIR1 cytoplasmic domain (amino acids 874-1205).

SEQ ID NO: 41 sets forth the nucleotide sequence of the AtRLP23+TM-AtSOBIR1-3×FLAG polynucleotide construct. The construct comprises a first nucleotide sequence encoding the apoplastic, eJM, and TM domains of AtRLP23 (nucleotides 1-2619) operably linked to a second nucleotide sequence encoding the AtSOBIR1 cytoplasmic domain (nucleotides 2620-3615), operably linked to third nucleotide sequence encoding a linking amino acid (nucleotides 3616-3618), operably linked to a fourth nucleotide sequence encoding the 3×FLAG peptide (nucleotides 3619-3799). If desired, a stop codon (e.g. TAA, TAG, or TGA) can be operably linked to the 3′ end of a nucleic acid molecule comprising SEQ ID NO: 41. It is noted that in the Examples below, the stop codon TGA was used with this construct.

SEQ ID NO: 42 sets forth the amino acid sequence of AtRLP23+TM-AtSOBIR1-3×FLAG protein encoded by SEQ ID NO: 41. The AtRLP23+TM-AtSOBIR1-3×FLAG protein comprises a first polypeptide comprising the AtRLP23 apoplastic, eJM, and TM domains (amino acids 1-873) operably linked to a second polypeptide comprising the AtSOBIR1 cytoplasmic domain (amino acids 874-1205), operably linked to a linking amino acid (amino acid 1206), operably linked to the 3×FLAG peptide (amino acids 1207-1233).

SEQ ID NO: 43 sets forth the nucleotide sequence of the AtRLP23-eJM(EEEE/ADQ−) polynucleotide construct. The construct comprises in operable linkage nucleotide sequences encoding the AtRLP23 apoplastic domain, a modified AtRLP23 eJM domain, the AtRLP23 TM domain, and the AtRLP23 cytoplasmic domain. Relative to the amino acid sequence of the wild-type AtRLP23 eJM domain, the modified AtRLP23 eJM domain comprises the substitution of D for E at amino acid 841 of wild-type AtRLP23, Q for E at amino acid 843, and EV for EEV at amino acids 844-846. If desired, a stop codon (e.g. TAA, TAG, or TGA) can be operably linked to the 3′ end of a nucleic acid molecule comprising SEQ ID NO: 43.

SEQ ID NO: 44 sets forth the amino acid sequence of AtRLP23-eJM(EEEE/ADQ−) protein encoded by SEQ ID NO: 43. The AtRLP23-eJM(EEEE/ADQ−) protein comprises the AtRLP23 apoplastic domain, a modified AtRLP23 eJM domain, the AtRLP23 TM domain, and the AtRLP23 cytoplasmic domain. Relative to the amino acid sequence of the wild-type AtRLP23 eJM domain, the modified AtRLP23 eJM domain comprises the substitution of D for E at amino acid 841 of wild-type AtRLP23, Q for E at amino acid 843, and EV for EEV at amino acids 844-846.

SEQ ID NO: 45 sets forth the nucleotide sequence of the AtRLP23-eJM(EEEE/ADQ−)-3×FLAG polynucleotide construct. The construct comprises the nucleotide sequence of SEQ ID NO: 43 (nucleotides 1-2667), operably linked to a nucleotide sequence encoding a linking amino acid (nucleotides 2668-2670), operably linked to a nucleotide sequence encoding the 3×FLAG peptide (nucleotides 2671-2751). If desired, a stop codon (e.g. TAA, TAG, or TGA) can be operably linked to the 3′ end of a nucleic acid molecule comprising SEQ ID NO: 45. It is noted that in the Examples below, the stop codon TGA was used with this construct.

SEQ ID NO: 46 sets forth the amino acid sequence of AtRLP23-eJM(EEEE/ADQ−)-3×FLAG protein encoded by SEQ ID NO: 45. The AtRLP23-eJM(EEEE/ADQ−)-3×FLAG protein comprises the amino acid sequence set forth in SEQ ID NO: 44 (amino acids 1-889), operably linked to a linking amino acid (amino acid 890), operably linked to the 3×FLAG peptide (amino acids 891-917).

SEQ ID NO: 47 sets forth the nucleotide sequence of the AtRLP23-eJMAtRLP1 polynucleotide construct. The construct comprises in operable linkage nucleotide sequences encoding the AtRLP23 apoplastic domain (nucleotides 1-2496), the AtRLP1 eJM domain (nucleotides 2497-2544), the AtRLP23 TM and cytoplasmic domains (nucleotides 2545-2664). If desired, a stop codon (e.g. TAA, TAG, or TGA) can be operably linked to the 3′ end of a nucleic acid molecule comprising SEQ ID NO: 47.

SEQ ID NO: 48 sets forth the amino acid sequence of AtRLP23-eJMAtRLP1 protein encoded by SEQ ID NO: 47. The AtRLP23-eJMAtRLP1 protein comprises the AtRLP23 apoplastic domain (amino acids 1-832), the AtRLP1 eJM domain (amino acids 833-848), and the AtRLP23 TM and cytoplasmic domains (amino acids 849-888).

SEQ ID NO: 49 sets forth the nucleotide sequence of the AtRLP23-eJMAtRLP1-3×FLAG polynucleotide construct. The construct comprises the nucleotide sequence of SEQ ID NO: 47 (nucleotides 1-2664), operably linked to a nucleotide sequence encoding a linking amino acid (nucleotides 2665-2667), operably linked to a nucleotide sequence encoding the 3×FLAG peptide (nucleotides 2668-2748). If desired, a stop codon (e.g. TAA, TAG, or TGA) can be operably linked to the 3′ end of a nucleic acid molecule comprising SEQ ID NO: 49. It is noted that in the Examples below, the stop codon TGA was used with this construct.

SEQ ID NO: 50 sets forth the amino acid sequence of AtRLP23-eJMAtRLP1-3×FLAG protein encoded by SEQ ID NO: 49. The AtRLP23-eJMAtRLP1-3×FLAG protein comprises the amino acid sequence set forth in SEQ ID NO: 48 (amino acids 1-888), operably linked to a linking amino acid (amino acid 889), operably linked to the 3×FLAG peptide (amino acids 890-916).

SEQ ID NO: 51 sets forth the nucleotide sequence of the AtRLP23-eJMVe1 polynucleotide construct. The construct comprises in operable linkage nucleotide sequences encoding the AtRLP23 apoplastic domain (nucleotides 1-2496), the SlVe1 eJM domain (nucleotides 2497-2547), the AtRLP23 TM and cytoplasmic domains (nucleotides 2548-2667). If desired, a stop codon (e.g. TAA, TAG, or TGA) can be operably linked to the 3′ end of a nucleic acid molecule comprising SEQ ID NO: 51.

SEQ ID NO: 52 sets forth the amino acid sequence of AtRLP23-eJMVe1 protein encoded by SEQ ID NO: 51. The AtRLP23-eJMVe1 protein comprises the AtRLP23 apoplastic domain (amino acids 1-832), the SlVe1 eJM domain (amino acids 833-849), and the AtRLP23 TM and cytoplasmic domains (amino acids 850-889).

SEQ ID NO: 53 sets forth the nucleotide sequence of the AtRLP23-eJMVe1-3×FLAG polynucleotide construct. The construct comprises the nucleotide sequence of SEQ ID NO: 51 (nucleotides 1-2667), operably linked to a nucleotide sequence encoding a linking amino acid (nucleotides 2668-2670), operably linked to a nucleotide sequence encoding the 3×FLAG peptide (nucleotides 2671-2751). If desired, a stop codon (e.g. TAA, TAG, or TGA) can be operably linked to the 3′ end of a nucleic acid molecule comprising SEQ ID NO: 53. It is noted that in the Examples below, the stop codon TGA was used with this construct.

SEQ ID NO: 54 sets forth the amino acid sequence of AtRLP23-eJMVe1-3×FLAG protein encoded by SEQ ID NO: 53. The AtRLP23-eJMVe1-3×FLAG protein comprises the amino acid sequence set forth in SEQ ID NO: 52 (amino acids 1-889), operably linked to a linking amino acid (amino acid 890), operably linked to the 3×FLAG peptide (amino acids 891-917).

SEQ ID NO: 55 sets forth the nucleotide sequence of the AtRLP23-eJMAtRLP42 polynucleotide construct. The construct comprises in operable linkage nucleotide sequences encoding the AtRLP23 apoplastic domain (nucleotides 1-2496), the AtRLP42 eJM domain (nucleotides 2497-2544), the AtRLP23 TM and cytoplasmic domains (nucleotides 2545-2664). If desired, a stop codon (e.g. TAA, TAG, or TGA) can be operably linked to the 3′ end of a nucleic acid molecule comprising SEQ ID NO: 55.

SEQ ID NO: 56 sets forth the amino acid sequence of AtRLP23-eJMAtRLP42 protein encoded by SEQ ID NO: 55. The AtRLP23-eJMAtRLP42 protein comprises the AtRLP23 apoplastic domain (amino acids 1-832), the AtRLP42 eJM domain (amino acids 833-848), and the AtRLP23 TM and cytoplasmic domains (amino acids 849-888).

SEQ ID NO: 57 sets forth the nucleotide sequence of the AtRLP23-eJMAtRLP42-3×FLAG polynucleotide construct. The construct comprises the nucleotide sequence of SEQ ID NO: 55 (nucleotides 1-2664), operably linked to a nucleotide sequence encoding a linking amino acid (nucleotides 2665-2667), operably linked to a nucleotide sequence encoding the 3×FLAG peptide (nucleotides 2668-2748). If desired, a stop codon (e.g. TAA, TAG, or TGA) can be operably linked to the 3′ end of a nucleic acid molecule comprising SEQ ID NO: 57. It is noted that in the Examples below, the stop codon TGA was used with this construct.

SEQ ID NO: 58 sets forth the amino acid sequence of AtRLP23-eJMAtRLP42-3×FLAG protein encoded by SEQ ID NO: 57. The AtRLP23-eJMAtRLP42-3×FLAG protein comprises the amino acid sequence set forth in SEQ ID NO: 56 (amino acids 1-888), operably linked to a linking amino acid (amino acid 889), operably linked to the 3×FLAG peptide (amino acids 890-916).

SEQ ID NO: 59 sets forth the nucleotide sequence of the AtRLP23-eJMAtRLP42-AtEFR polynucleotide construct. The construct comprises in operable linkage nucleotide sequences encoding the AtRLP23 apoplastic domain (nucleotides 1-2496), the AtRLP42 eJM domain (nucleotides 2497-2544), the AtRLP23 TM and the first part of two parts of the cytoplasmic domain (nucleotides 2545-3298), an AtEFR intron (nucleotides 3299-3385), and the second part of the cytoplasmic domain (nucleotides 3386-3777). If desired, a stop codon (e.g. TAA, TAG, or TGA) can be operably linked to the 3′ end of a nucleic acid molecule comprising SEQ ID NO: 59.

SEQ ID NO: 60 sets forth the amino acid sequence of AtRLP23-eJMAtRLP42-AtEFR protein encoded by SEQ ID NO: 59. The AtRLP23-eJMAtRLP42-AtEFR protein comprises the AtRLP23 apoplastic domain (amino acids 1-832), the AtRLP42 eJM domain (amino acids 833-848), and the AtEFR TM and cytoplasmic domains (amino acids 849-1230).

SEQ ID NO: 61 sets forth the nucleotide sequence of the AtRLP23-eJMAtRLP42-AtEFR-3×FLAG polynucleotide construct. The construct comprises the nucleotide sequence of SEQ ID NO: 59 (nucleotides 1-3777), operably linked to a nucleotide sequence encoding a linker dipeptide (nucleotides 3778-3783), operably linked to a nucleotide sequence encoding the 3×FLAG peptide (nucleotides 3784-3864). If desired, a stop codon (e.g. TAA, TAG, or TGA) can be operably linked to the 3′ end of a nucleic acid molecule comprising SEQ ID NO: 61. It is noted that in the Examples below, the stop codon TGA was used with this construct.

SEQ ID NO: 62 sets forth the amino acid sequence of AtRLP23-eJMAtRLP42-AtEFR-3×FLAG protein encoded by SEQ ID NO: 61. The AtRLP23-eJMAtRLP42-AtEFR-3×FLAG protein comprises the amino acid sequence set forth in SEQ ID NO: 60 (amino acids 1-1230), operably linked to a linker dipeptide (amino acids 1231-1232), operably linked to the 3×FLAG peptide (amino acids 1233-1259).

SEQ ID NO: 63 sets forth the amino acid sequence of the PpNLP20 peptide from Phytophthora parasitica.

SEQ ID NO: 64 sets forth the amino acid sequence of the CgNLP24b peptide from Colletotrichum graminicola M1.001.

SEQ ID NO: 65 sets forth the amino acid sequence of the FgNLP24c peptide from Fusarium graminearum PH-1.

SEQ ID NO: 66 sets forth the amino acid sequence of the FvNLP24a peptide from Fusarium verticillioides 7600.

SEQ ID NO: 67 sets forth the amino acid sequence of the SmNLP24 peptide from Stenocarpella maydis A1-1.

SEQ ID NO: 68 sets forth the amino acid sequence of the AfNLP24a peptide from Aspergillus flavus NRRL3357.

SEQ ID NO: 69 sets forth the amino acid sequence of the AfNLP24b peptide from Aspergillus flavus NRRL3357.

SEQ ID NO: 70 sets forth the amino acid sequence of the AfNLP24c peptide from Aspergillus flavus NRRL3357.

SEQ ID NO: 71 sets forth the amino acid sequence of the ApNLP24a peptide from Aspergillus parasiticus SU-1.

SEQ ID NO: 72 sets forth the nucleotide sequence of the 2×355+Omega (Ω) promoter construct. The construct comprises in operable linkage two copies of the Cauliflower Mosaic Virus 35S (CaMV 35S) promoter (nucleotides 1-327 and 328-653) and the 5′-untranslated region (UTR) omega (Ω) region of from Tobacco Mosaic Virus (nucleotides 627-828).

SEQ ID NO: 73 sets forth the nucleotide sequence of the 3′ UTR of the small subunit ribulose bisphosphate carboxylase/oxygenase E9 gene from pea (Pisum sativum) (referred to here as “rbcS termination region”, “rbcS terminator”, or simply “rbcS”).

SEQ ID NO: 74 sets forth the nucleotide sequence of the circular pUC19 vector.

SEQ ID NO: 75 sets forth the nucleotide sequence of the circular ZmUbi::R-GECO1.2::rbcS vector construct. The construct comprises in operable linkage the Zea mays ubiquitin promoter (ZmUbi) (nucleotides 5-899), the first of two parts of the Z. mays ubiquitin 5′-UTR (nucleotides 900-981), an intron from the Z. mays ubiquitin gene that is found in this same position within the Z. mays ubiquitin 5′-UTR (nucleotides 982-1991), and the second part of the Z. mays ubiquitin 5′-UTR (nucleotides 1992-1993), a coding sequence for R-GECO1.2 (nucleotides 1994-3247), and the rbcS termination region (nucleotides 3252-3893).

SEQ ID NO: 76 sets forth the nucleotide sequence of the circular ZmUbi::Aequorin::rbcS vector construct. The construct comprises in operable linkage the Zea mays ubiquitin promoter (ZmUbi) (nucleotides 5-899), the first of two parts of the Z. mays ubiquitin 5′-UTR (nucleotides 900-981), an intron from the Z. mays ubiquitin gene that is found in this same position within the Z. mays ubiquitin 5′-UTR (nucleotides 982-1991), and the second part of the Z. mays ubiquitin 5′-UTR (nucleotides 1992-1993), a coding sequence for Aequorin (nucleotides 1994-2584), and the rbcS termination region (nucleotides 2589-3230).

Each of SEQ ID NOS: 77-240 and 561-563 sets forth the amino acid sequence of a kinase domain from a plant receptor-like kinases (RLK) from Arabidopsis thaliana, Medicago truncatula, Oryza sativa, or Solanum lycopersicum that can be used in the methods and compositions of present invention as described elsewhere herein. In the following descriptions for each of these sequences, the RLK subgroup (if assigned or otherwise known) and the plant species from which each sequence the RLK was derived is provided in parentheses.

SEQ ID NO: 77 sets forth the amino acid sequence of the kinase domain from the RLK protein encoded by gene locus AT1g01740 (RLCK; Arabidopsis thaliana).

SEQ ID NO: 78 sets forth the amino acid sequence of the kinase domain from the RLK protein encoded by gene locus AT1g80640 (RLCK; Arabidopsis thaliana).

SEQ ID NO: 79 sets forth the amino acid sequence of the kinase domain from the RLK protein encoded by gene locus AT3g59350 (RLCK; Arabidopsis thaliana).

SEQ ID NO: 80 sets forth the amino acid sequence of the kinase domain from the RLK protein encoded by gene locus AT4g35230 (RLCK; Arabidopsis thaliana).

SEQ ID NO: 81 sets forth the amino acid sequence of the kinase domain from the RLK protein encoded by gene locus AT5G13160 (RLCK; Arabidopsis thaliana).

SEQ ID NO: 82 sets forth the amino acid sequence of the kinase domain from the RLK protein encoded by gene locus AT5g57670 (RLCK; Arabidopsis thaliana).

SEQ ID NO: 83 sets forth the amino acid sequence of the kinase domain from the RLK protein encoded by gene locus AT5g58940 (RLCK; Arabidopsis thaliana).

SEQ ID NO: 84 sets forth the amino acid sequence of the kinase domain from the RLK protein encoded by gene locus AT2G39180 (CR4L; Arabidopsis thaliana).

SEQ ID NO: 85 sets forth the amino acid sequence of the kinase domain from the RLK protein encoded by gene locus LOC_Os08g01830 (CR4L; Oryza sativa).

SEQ ID NO: 86 sets forth the amino acid sequence of the kinase domain from the RLK protein encoded by gene locus Medtr1g064560 (CR4L; Medicago truncatula).

SEQ ID NO: 87 sets forth the amino acid sequence of the kinase domain from the RLK protein encoded by gene locus Solyc09g007750.2 (CR4L; Solanum lycopersicum).

SEQ ID NO: 88 sets forth the amino acid sequence of the kinase domain from the RLK protein encoded by gene locus Solyc07g049180.2 (LysM; Solanum lycopersicum).

SEQ ID NO: 89 sets forth the amino acid sequence of the kinase domain from the RLK protein encoded by gene locus AT3G24550 (PERK; Arabidopsis thaliana).

SEQ ID NO: 90 sets forth the amino acid sequence of the kinase domain from the RLK protein encoded by gene locus LOC_Os03g16260 (PERK; Oryza sativa).

SEQ ID NO: 91 sets forth the amino acid sequence of the kinase domain from the RLK protein encoded by gene locus LOC_Os05g12680 (PERK; Oryza sativa).

SEQ ID NO: 92 sets forth the amino acid sequence of the kinase domain from the RLK protein encoded by gene locus Medtr5g034210 (PERK; Medicago truncatula).

SEQ ID NO: 93 sets forth the amino acid sequence of the kinase domain from the RLK protein encoded by gene locus Medtr6g088610 (PERK; Medicago truncatula).

SEQ ID NO: 94 sets forth the amino acid sequence of the kinase domain from the RLK protein encoded by gene locus Solyc10g051330.1 (PERK; Solanum lycopersicum).

SEQ ID NO: 95 sets forth the amino acid sequence of the kinase domain from the RLK protein encoded by gene locus Solyc12g007110.1 (PERK; Solanum lycopersicum).

SEQ ID NO: 96 sets forth the amino acid sequence of the kinase domain from the RLK protein encoded by gene locus AT1G11050 (RKF3; Arabidopsis thaliana).

SEQ ID NO: 97 sets forth the amino acid sequence of the kinase domain from the RLK protein encoded by gene locus LOC_Os05g34950 (RKF3; Oryza sativa).

SEQ ID NO: 98 sets forth the amino acid sequence of the kinase domain from the RLK protein encoded by gene locus Medtr2g006910 (RKF3; Medicago truncatula).

SEQ ID NO: 99 sets forth the amino acid sequence of the kinase domain from the RLK protein encoded by gene locus Solyc01g104050.2 (RKF3; Solanum lycopersicum).

SEQ ID NO: 100 sets forth the amino acid sequence of the kinase domain from the RLK protein encoded by gene locus AT3G28040 (LRR-VIIa; Arabidopsis thaliana).

SEQ ID NO: 101 sets forth the amino acid sequence of the kinase domain from the RLK protein encoded by gene locus LOC_Os01g72700 (LRR-VIIa; Oryza sativa).

SEQ ID NO: 102 sets forth the amino acid sequence of the kinase domain from the RLK protein encoded by gene locus Medtr7g022160v2 (LRR-VIIa; Medicago truncatula).

SEQ ID NO: 103 sets forth the amino acid sequence of the kinase domain from the RLK protein encoded by gene locus Solyc09g098290.2 (LRR-VIIa; Solanum lycopersicum).

SEQ ID NO: 104 sets forth the amino acid sequence of the kinase domain from the RLK protein encoded by gene locus AT1G66980 (LRK10L-2/GDPD; Arabidopsis thaliana).

SEQ ID NO: 105 sets forth the amino acid sequence of the kinase domain from the RLK protein encoded by gene locus AT1g49730 (URK; Arabidopsis thaliana).

SEQ ID NO: 106 sets forth the amino acid sequence of the kinase domain from the RLK protein encoded by gene locus Medtr3g075440 (unassigned; Medicago truncatula).

SEQ ID NO: 107 sets forth the amino acid sequence of the kinase domain from the RLK protein (WAK1) encoded by gene locus AT1G21250 (WAK; Arabidopsis thaliana).

SEQ ID NO: 108 sets forth the amino acid sequence of the kinase domain from the RLK protein encoded by gene locus LOC_Os04g51040 (WAK; Oryza sativa).

SEQ ID NO: 109 sets forth the amino acid sequence of the kinase domain from the RLK protein encoded by gene locus LOC_Os04g51050 (WAK5; Oryza sativa).

SEQ ID NO: 110 sets forth the amino acid sequence of the kinase domain from the RLK protein encoded by gene locus Medtr1g010250 (WAK; Medicago truncatula).

SEQ ID NO: 111 sets forth the amino acid sequence of the kinase domain from the RLK protein encoded by gene locus Medtr1g027670 (WAK; Medicago truncatula).

SEQ ID NO: 112 sets forth the amino acid sequence of the kinase domain from the RLK protein encoded by gene locus Medtr6g083020 (WAK; Medicago truncatula).

SEQ ID NO: 113 sets forth the amino acid sequence of the kinase domain from the RLK protein encoded by gene locus OsXa21 (LRR; Oryza sativa).

SEQ ID NO: 114 sets forth the amino acid sequence of the kinase domain from the RLK protein encoded by gene locus Solyc09g015240.1 (WAK; Solanum lycopersicum).

SEQ ID NO: 115 sets forth the amino acid sequence of the kinase domain from the RLK protein encoded by gene locus Solyc11g072140.1 (WAK; Solanum lycopersicum).

SEQ ID NO: 116 sets forth the amino acid sequence of the kinase domain from the RLK protein encoded by gene locus AT1G18390v2 (LRK10L-1; Arabidopsis thaliana).

SEQ ID NO: 117 sets forth the amino acid sequence of the kinase domain from the RLK protein encoded by gene locus AT5G38210v2 (LRK10L-1; Arabidopsis thaliana).

SEQ ID NO: 118 sets forth the amino acid sequence of the kinase domain from the RLK protein encoded by gene locus Solyc02g086210.2 (LRK10L-2; Solanum lycopersicum).

SEQ ID NO: 119 sets forth the amino acid sequence of the kinase domain from the RLK protein encoded by gene locus Medtr7g082470v2 (WAK; Medicago truncatula).

SEQ ID NO: 120 sets forth the amino acid sequence of the kinase domain from the RLK protein encoded by gene locus Solyc03g119240.2 (WAK; Solanum lycopersicum).

SEQ ID NO: 121 sets forth the amino acid sequence of the kinase domain from the RLK protein encoded by gene locus AT1G66930v2 (LRK10L-2/WAK; Arabidopsis thaliana).

SEQ ID NO: 122 sets forth the amino acid sequence of the kinase domain from the RLK protein encoded by gene locus LOC_Os01g49614 (LRK10L-2/WAK; Oryza sativa).

SEQ ID NO: 123 sets forth the amino acid sequence of the kinase domain from the RLK protein encoded by gene locus AT4G23190 (DUF26-Ib; Arabidopsis thaliana).

SEQ ID NO: 124 sets forth the amino acid sequence of the kinase domain from the RLK protein encoded by gene locus Medtr1g105650 (DUF26-Ib; Medicago truncatula).

SEQ ID NO: 125 sets forth the amino acid sequence of the kinase domain from the RLK protein encoded by gene locus Solyc02g080040.2 (DUF26-Ib; Solanum lycopersicum).

SEQ ID NO: 126 sets forth the amino acid sequence of the kinase domain from the RLK protein encoded by gene locus LOC_Os07g35310 (DUF26-Ig; Oryza sativa).

SEQ ID NO: 127 sets forth the amino acid sequence of the kinase domain from the RLK protein encoded by gene locus LOC_Os01g04409 (WAK; Oryza sativa).

SEQ ID NO: 128 sets forth the amino acid sequence of the kinase domain from the RLK protein encoded by gene locus AT1G52310 (C-LEC; Arabidopsis thaliana).

SEQ ID NO: 129 sets forth the amino acid sequence of the kinase domain from the RLK protein encoded by gene locus LOC_Os01g01410 (C-LEC; Oryza sativa).

SEQ ID NO: 130 sets forth the amino acid sequence of the kinase domain from the RLK protein encoded by gene locus Medtr2g043380 (C-Lec; Medicago truncatula).

SEQ ID NO: 131 sets forth the amino acid sequence of the kinase domain from the RLK protein encoded by gene locus Solyc02g068370.2 (C-Lec; Solanum lycopersicum).

SEQ ID NO: 132 sets forth the amino acid sequence of the kinase domain from the RLK protein encoded by gene locus AT4G02010v2 (Extensin; Arabidopsis thaliana).

SEQ ID NO: 133 sets forth the amino acid sequence of the kinase domain from the RLK protein encoded by gene locus AT5G56890 (Extensin; Arabidopsis thaliana).

SEQ ID NO: 134 sets forth the amino acid sequence of the kinase domain from the RLK protein encoded by gene locus LOC_Os01g14932 (Extensin; Oryza sativa).

SEQ ID NO: 135 sets forth the amino acid sequence of the kinase domain from the RLK protein encoded by gene locus LOC_Os01g51290 (Extensin; Oryza sativa).

SEQ ID NO: 136 sets forth the amino acid sequence of the kinase domain from the RLK protein encoded by gene locus LOC_Os05g11750 (Extensin; Oryza sativa).

SEQ ID NO: 137 sets forth the amino acid sequence of the kinase domain from the RLK protein encoded by gene locus LOC_Os05g33080 (Extensin; Oryza sativa).

SEQ ID NO: 138 sets forth the amino acid sequence of the kinase domain from the RLK protein encoded by gene locus Medtr1g082580 (Extensin; Medicago truncatula).

SEQ ID NO: 139 sets forth the amino acid sequence of the kinase domain from the RLK protein encoded by gene locus Medtr2g039290 (Extensin; Medicago truncatula).

SEQ ID NO: 140 sets forth the amino acid sequence of the kinase domain from the RLK protein encoded by gene locus Medtr2g084120 (Extensin; Medicago truncatula).

SEQ ID NO: 141 sets forth the amino acid sequence of the kinase domain from the RLK protein encoded by gene locus Medtr7g109670 (Extensin; Medicago truncatula).

SEQ ID NO: 142 sets forth the amino acid sequence of the kinase domain from the RLK protein encoded by gene locus Solyc01g079340.2 (Extensin; Solanum lycopersicum).

SEQ ID NO: 143 sets forth the amino acid sequence of the kinase domain from the RLK protein encoded by gene locus Solyc07g039340.2 (Extensin; Solanum lycopersicum).

SEQ ID NO: 144 sets forth the amino acid sequence of the kinase domain from the RLK protein encoded by gene locus Solyc07g055180.2 (Extensin; Solanum lycopersicum).

SEQ ID NO: 145 sets forth the amino acid sequence of the kinase domain from the RLK protein encoded by gene locus AT1G65790 (SD-1a/G-Lec; Arabidopsis thaliana).

SEQ ID NO: 146 sets forth the amino acid sequence of the kinase domain from the RLK protein encoded by gene locus AT2G41890 (SD-3/G-Lec; Arabidopsis thaliana).

SEQ ID NO: 147 sets forth the amino acid sequence of the kinase domain from the RLK protein encoded by gene locus AT4G00340 (SD-2b/G-Lec; Arabidopsis thaliana).

SEQ ID NO: 148 sets forth the amino acid sequence of the kinase domain from the RLK protein encoded by gene locus LOC_Os01g45520 (SD-2b/G-Lec; Oryza sativa).

SEQ ID NO: 149 sets forth the amino acid sequence of the kinase domain from the RLK protein encoded by gene locus LOC_Os03g35600 (SD-1a/G-Lec; Oryza sativa).

SEQ ID NO: 150 sets forth the amino acid sequence of the kinase domain from the RLK protein encoded by gene locus LOC_Os07g36780 (SD-2b/G-Lec; Oryza sativa).

SEQ ID NO: 151 sets forth the amino acid sequence of the kinase domain from the RLK protein encoded by gene locus Medtr0280s0040 (SD-2b/G-Lec; Medicago truncatula).

SEQ ID NO: 152 sets forth the amino acid sequence of the kinase domain from the RLK protein encoded by gene locus Medtr4g091670 (SD-1a/G-Lec; Medicago truncatula).

SEQ ID NO: 153 sets forth the amino acid sequence of the kinase domain from the RLK protein encoded by gene locus Medtr7g092050 (SD-3/G-Lec; Medicago truncatula).

SEQ ID NO: 154 sets forth the amino acid sequence of the kinase domain from the RLK protein encoded by gene locus Medtr8g030500 (SD-2b/G-Lec; Medicago truncatula).

SEQ ID NO: 155 sets forth the amino acid sequence of the kinase domain from the RLK protein encoded by gene locus Solyc01g094830.2v2 (SD-2b/G-Lec; Solanum lycopersicum).

SEQ ID NO: 156 sets forth the amino acid sequence of the kinase domain from the RLK protein encoded by gene locus Solyc02g079590.2 (SD-1a/G-Lec; Solanum lycopersicum).

SEQ ID NO: 157 sets forth the amino acid sequence of the kinase domain from the RLK protein encoded by gene locus Solyc03g063650.1 (SD-3/G-Lec; Solanum lycopersicum).

SEQ ID NO: 158 sets forth the amino acid sequence of the kinase domain from the RLK protein encoded by gene locus AT2G31880 (unassigned; Arabidopsis thaliana) which is the locus that encodes AtSOBIR1. The full-length AtSOBIR1 amino acid sequence is set forth in SEQ ID NO: 8.

SEQ ID NO: 159 sets forth the amino acid sequence of the kinase domain from the RLK protein encoded by gene locus LOC_Os01g72990 (LRR-VIII-1; Oryza sativa).

SEQ ID NO: 160 sets forth the amino acid sequence of the kinase domain from the RLK protein encoded by gene locus LOC_Os05g40770 (LRR-VIII-1; Oryza sativa).

SEQ ID NO: 161 sets forth the amino acid sequence of the kinase domain from the RLK protein encoded by gene locus LOC_Os12g10740 (LRR-VIII-1; Oryza sativa).

SEQ ID NO: 162 sets forth the amino acid sequence of the kinase domain from the RLK protein encoded by gene locus Medtr3g062590 (LRR-VIII-1; Medicago truncatula).

SEQ ID NO: 163 sets forth the amino acid sequence of the kinase domain from the RLK protein encoded by gene locus Medtr8g070880 (LRR-VIII-1; Medicago truncatula).

SEQ ID NO: 164 sets forth the amino acid sequence of the kinase domain from the RLK protein encoded by gene locus AT3G14840 (LRR-VIII-2; Arabidopsis thaliana).

SEQ ID NO: 165 sets forth the amino acid sequence of the kinase domain from the RLK protein encoded by gene locus LOC_Os09g17630 (LRR-VIII-2; Oryza sativa).

SEQ ID NO: 166 sets forth the amino acid sequence of the kinase domain from the RLK protein encoded by gene locus LOC_Os12g41710 (LRR-VIII-2; Oryza sativa).

SEQ ID NO: 167 sets forth the amino acid sequence of the kinase domain from the RLK protein encoded by gene locus Medtr2g075010 (LRR-VIII-2; Medicago truncatula).

SEQ ID NO: 168 sets forth the amino acid sequence of the kinase domain from the RLK protein encoded by gene locus Solyc12g014350.1 (LRR-VIII-2; Solanum lycopersicum).

SEQ ID NO: 169 sets forth the amino acid sequence of the kinase domain from the RLK protein encoded by gene locus AT3G51550 (CrRLK1L-1; Arabidopsis thaliana).

SEQ ID NO: 170 sets forth the amino acid sequence of the kinase domain from the RLK protein encoded by gene locus LOC_Os03g21540 (CrRLK1L-1; Oryza sativa).

SEQ ID NO: 171 sets forth the amino acid sequence of the kinase domain from the RLK protein encoded by gene locus Medtr7g073660 (CrRLK1L-1; Medicago truncatula).

SEQ ID NO: 172 sets forth the amino acid sequence of the kinase domain from the RLK protein encoded by gene locus AT3G08870 (L-LEC; Arabidopsis thaliana).

SEQ ID NO: 173 sets forth the amino acid sequence of the kinase domain from the RLK protein encoded by gene locus AT3G59700 (L-LEC; Arabidopsis thaliana).

SEQ ID NO: 174 sets forth the amino acid sequence of the kinase domain from the RLK protein encoded by gene locus LOC_Os07g03830 (L-Lec; Oryza sativa).

SEQ ID NO: 175 sets forth the amino acid sequence of the kinase domain from the RLK protein encoded by gene locus Medtr7g062990 (L-Lec; Medicago truncatula).

SEQ ID NO: 176 sets forth the amino acid sequence of the kinase domain from the RLK protein encoded by gene locus Solyc05g053010.1 (L-Lec; Solanum lycopersicum).

SEQ ID NO: 177 sets forth the amino acid sequence of the kinase domain from the RLK protein encoded by gene locus Solyc06g009550.2 (CrRLK1L1; Solanum lycopersicum).

SEQ ID NO: 178 sets forth the amino acid sequence of the kinase domain from the RLK protein encoded by gene locus AT1G53730 (LRR-V; Arabidopsis thaliana).

SEQ ID NO: 179 sets forth the amino acid sequence of the kinase domain from the RLK protein encoded by gene locus LOC_Os03g08550 (LRR-V; Oryza sativa).

SEQ ID NO: 180 sets forth the amino acid sequence of the kinase domain from the RLK protein encoded by gene locus Medtr7g117520 (LRR-V; Medicago truncatula).

SEQ ID NO: 181 sets forth the amino acid sequence of the kinase domain from the RLK protein encoded by gene locus Solyc03g123740.2 (LRR-V; Solanum lycopersicum).

SEQ ID NO: 182 sets forth the amino acid sequence of the kinase domain from the RLK protein encoded by gene locus AT5G38280 (LRK10L-2/thaumatin; Arabidopsis thaliana).

SEQ ID NO: 183 sets forth the amino acid sequence of the kinase domain from the RLK protein encoded by gene locus AT1G48480 (LRR-III; Arabidopsis thaliana).

SEQ ID NO: 184 sets forth the amino acid sequence of the kinase domain from the RLK protein encoded by gene locus LOC_Os03g50450 (LRR-III; Oryza sativa).

SEQ ID NO: 185 sets forth the amino acid sequence of the kinase domain from the RLK protein encoded by gene locus Medtr8g107470 (LRR-III; Medicago truncatula).

SEQ ID NO: 186 sets forth the amino acid sequence of the kinase domain from the RLK protein encoded by gene locus Solyc03g118510.2 (LRR-III; Solanum lycopersicum).

SEQ ID NO: 187 sets forth the amino acid sequence of the kinase domain from the RLK protein encoded by gene locus LOC_Os06g18000 (unassigned; Oryza sativa).

SEQ ID NO: 188 sets forth the amino acid sequence of the kinase domain from the RLK protein encoded by gene locus AT3G47090 (LRR-XII; Arabidopsis thaliana).

SEQ ID NO: 189 sets forth the amino acid sequence of the kinase domain from the RLK protein encoded by gene locus AT5G20480 (LRR-XII-AtEFR; Arabidopsis thaliana), which is the locus that encodes AtEFR. The full-length AtEFR amino acid sequence is set forth in SEQ ID NO: 6.

SEQ ID NO: 190 sets forth the amino acid sequence of the kinase domain from the RLK protein encoded by gene locus LOC_Os12g42520 (LRR-XII; Oryza sativa).

SEQ ID NO: 191 sets forth the amino acid sequence of the kinase domain from the RLK protein encoded by gene locus Medtr5g019070v2 (LRR-XII; Medicago truncatula).

SEQ ID NO: 192 sets forth the amino acid sequence of the kinase domain from the RLK protein encoded by gene locus Medtr5g044680 (LRR-XII; Medicago truncatula).

SEQ ID NO: 193 sets forth the amino acid sequence of the kinase domain from the RLK protein encoded by gene locus Solyc03g019980.1v2 (LRR-XII; Solanum lycopersicum).

SEQ ID NO: 194 sets forth the amino acid sequence of the kinase domain from the RLK protein encoded by gene locus Solyc06g076910.1 (LRR-XII; Solanum lycopersicum).

SEQ ID NO: 195 sets forth the amino acid sequence of the kinase domain from the RLK protein encoded by gene locus AT2G16250 (LRR-XIV; Arabidopsis thaliana).

SEQ ID NO: 196 sets forth the amino acid sequence of the kinase domain from the RLK protein encoded by gene locus LOC_Os03g51440 (LRR-XIV; Oryza sativa).

SEQ ID NO: 197 sets forth the amino acid sequence of the kinase domain from the RLK protein encoded by gene locus Medtr3g116640 (LRR-XIV; Medicago truncatula).

SEQ ID NO: 198 sets forth the amino acid sequence of the kinase domain from the RLK protein encoded by gene locus Solyc01g109650.2 (LRR-XIV; Solanum lycopersicum).

SEQ ID NO: 199 sets forth the amino acid sequence of the kinase domain from the RLK protein encoded by gene locus AT3G46420 (LRR-Ia; Arabidopsis thaliana).

SEQ ID NO: 200 sets forth the amino acid sequence of the kinase domain from the RLK protein encoded by gene locus LOC_Os05g44970 (LRR-Ia; Oryza sativa).

SEQ ID NO: 201 sets forth the amino acid sequence of the kinase domain from the RLK protein encoded by gene locus Medtr8g015340 (LRR-Ia; Medicago truncatula).

SEQ ID NO: 202 sets forth the amino acid sequence of the kinase domain from the RLK protein encoded by gene locus Solyc03g121230.2 (LRR-Ia; Solanum lycopersicum).

SEQ ID NO: 203 sets forth the amino acid sequence of the kinase domain from the RLK protein encoded by gene locus AT4G33430.1 (LRR-II; Arabidopsis thaliana).

SEQ ID NO: 204 sets forth the amino acid sequence of the kinase domain from the RLK protein encoded by gene locus LOC_Os08g07760 (LRR-II; Oryza sativa).

SEQ ID NO: 205 sets forth the amino acid sequence of the kinase domain from the RLK protein encoded by gene locus Medtr2g008360 (LRR-II; Medicago truncatula).

SEQ ID NO: 206 sets forth the amino acid sequence of the kinase domain from the RLK protein encoded by gene locus Solyc04g039730.2 (LRR-II; Solanum lycopersicum).

SEQ ID NO: 207 sets forth the amino acid sequence of the kinase domain from the RLK protein encoded by gene locus Solyc10g047140.1 (LRR-II; Solanum lycopersicum).

SEQ ID NO: 208 sets forth the amino acid sequence of the kinase domain from the RLK protein encoded by gene locus AT2G45340 (LRR-IV; Arabidopsis thaliana).

SEQ ID NO: 209 sets forth the amino acid sequence of the kinase domain from the RLK protein encoded by gene locus LOC_Os06g04370 (LRR-IV; Oryza sativa).

SEQ ID NO: 210 sets forth the amino acid sequence of the kinase domain from the RLK protein encoded by gene locus Medtr3g071480 (LRR-IV; Medicago truncatula).

SEQ ID NO: 211 sets forth the amino acid sequence of the kinase domain from the RLK protein encoded by gene locus Medtr6g060230 (LRR-IV; Medicago truncatula).

SEQ ID NO: 212 sets forth the amino acid sequence of the kinase domain from the RLK protein encoded by gene locus Solyc01g091230.2 (LRR-IV; Solanum lycopersicum).

SEQ ID NO: 213 sets forth the amino acid sequence of the kinase domain from the RLK protein encoded by gene locus AT1G66150 (LRR-IX; Arabidopsis thaliana).

SEQ ID NO: 214 sets forth the amino acid sequence of the kinase domain from the RLK protein encoded by gene locus LOC_Os03g50810 (LRR-IX; Oryza sativa).

SEQ ID NO: 215 sets forth the amino acid sequence of the kinase domain from the RLK protein encoded by gene locus Medtr5g077430 (LRR-IX; Medicago truncatula).

SEQ ID NO: 216 sets forth the amino acid sequence of the kinase domain from the RLK protein encoded by gene locus Solyc09g092460.2 (LRR-IX; Solanum lycopersicum).

SEQ ID NO: 217 sets forth the amino acid sequence of the kinase domain from the RLK protein encoded by gene locus Solyc11g006040.1 (LRR-IX; Solanum lycopersicum).

SEQ ID NO: 218 sets forth the amino acid sequence of the kinase domain from the RLK protein encoded by gene locus AT2G40270 (LRR-VI; Arabidopsis thaliana).

SEQ ID NO: 219 sets forth the amino acid sequence of the kinase domain from the RLK protein encoded by gene locus AT5G07150 (LRR-VI; Arabidopsis thaliana).

SEQ ID NO: 220 sets forth the amino acid sequence of the kinase domain from the RLK protein encoded by gene locus LOC_Os03g18370 (LRR-VI; Oryza sativa).

SEQ ID NO: 221 sets forth the amino acid sequence of the kinase domain from the RLK protein encoded by gene locus Solyc06g051030.2 (LRR-VI; Solanum lycopersicum).

SEQ ID NO: 222 sets forth the amino acid sequence of the kinase domain from the RLK protein encoded by gene locus AT4G39400 (LRR-Xb; Arabidopsis thaliana).

SEQ ID NO: 223 sets forth the amino acid sequence of the kinase domain from the RLK protein encoded by gene locus LOC_Os09g12240 (LRR-Xb; Oryza sativa).

SEQ ID NO: 224 sets forth the amino acid sequence of the kinase domain from the RLK protein encoded by gene locus Medtr3g095100 (LRR-Xb; Medicago truncatula).

SEQ ID NO: 225 sets forth the amino acid sequence of the kinase domain from the RLK protein encoded by gene locus Solyc04g051510.1 (LRR-Xb; Solanum lycopersicum).

SEQ ID NO: 226 sets forth the amino acid sequence of the kinase domain from the RLK protein encoded by gene locus AT1G75820 (LRR-XI; Arabidopsis thaliana).

SEQ ID NO: 227 sets forth the amino acid sequence of the kinase domain from the RLK protein encoded by gene locus LOC_Os03g56270 (LRR-XI; Oryza sativa).

SEQ ID NO: 228 sets forth the amino acid sequence of the kinase domain from the RLK protein encoded by gene locus Medtr4g070970 (LRR-XI; Medicago truncatula).

SEQ ID NO: 229 sets forth the amino acid sequence of the kinase domain from the RLK protein encoded by gene locus Solyc04g081590.2 (LRR-XI; Solanum lycopersicum).

SEQ ID NO: 230 sets forth the amino acid sequence of the kinase domain from the RLK protein encoded by gene locus Solyc06g071810.1 (LRR-XV; Solanum lycopersicum).

SEQ ID NO: 231 sets forth the amino acid sequence of the kinase domain from the RLK protein encoded by gene locus LOC_Os04g59320 (URK-I; Oryza sativa).

SEQ ID NO: 232 sets forth the amino acid sequence of the kinase domain from the RLK protein encoded by gene locus Medtr4g085810 (URK-I; Medicago truncatula).

SEQ ID NO: 233 sets forth the amino acid sequence of the kinase domain from the RLK protein encoded by gene locus Solyc01g108000.2 (URK-I; Solanum lycopersicum).

SEQ ID NO: 234 sets forth the amino acid sequence of the kinase domain from the RLK protein encoded by gene locus AT2G26330 (LRR-XIIIb; Arabidopsis thaliana).

SEQ ID NO: 235 sets forth the amino acid sequence of the kinase domain from the RLK protein encoded by gene locus LOC_Os02g53720 (LRR-XIIIb; Oryza sativa).

SEQ ID NO: 236 sets forth the amino acid sequence of the kinase domain from the RLK protein encoded by gene locus Medtr1g015530 (LRR-XIIIb; Medicago truncatula).

SEQ ID NO: 237 sets forth the amino acid sequence of the kinase domain from the RLK protein encoded by gene locus Solyc08g061560.2 (LRR-XIIIb; Solanum lycopersicum).

SEQ ID NO: 238 sets forth the amino acid sequence of the kinase domain from the RLK protein encoded by gene locus AT3G21630 (LysM-I; Arabidopsis thaliana).

SEQ ID NO: 239 sets forth the amino acid sequence of the kinase domain from the RLK protein (OsCERK1) encoded by gene locus LOC_Os08g42580 (LysM-I; Oryza sativa).

SEQ ID NO: 240 sets forth the amino acid sequence of the kinase domain from the RLK protein encoded by gene locus Medtr3g080050 (LysM-I; Medicago truncatula).

Each of SEQ ID NOS: 241-390, 541-547, and 564-566 sets forth the amino acid sequence of extra-juxtamembrane (eJM) domain from a plant receptor-like kinases (RLK) from Arabidopsis thaliana, Medicago truncatula, Oryza sativa, or Solanum lycopersicum that can be used in the methods and compositions of present invention as described elsewhere herein.

SEQ ID NO: 241 sets forth the amino acid sequence of the eJM domain from the RLK protein encoded by gene locus Medtr7g062990 of Medicago truncatula.

SEQ ID NO: 242 sets forth the amino acid sequence of the eJM domain from the RLK protein encoded by gene locus LOC_Os07g03830 of Oryza sativa.

SEQ ID NO: 243 sets forth the amino acid sequence of the eJM domain from the RLK protein encoded by gene locus AT3G08870 of Arabidopsis thaliana.

SEQ ID NO: 244 sets forth the amino acid sequence of the eJM domain from the RLK protein encoded by gene locus LOC_Os03g35600 of Oryza sativa.

SEQ ID NO: 245 sets forth the amino acid sequence of the eJM domain from the RLK protein encoded by gene locus Medtr4g091670 of Medicago truncatula.

SEQ ID NO: 246 sets forth the amino acid sequence of the eJM domain from the RLK protein encoded by gene locus AT1G65790 of Arabidopsis thaliana.

SEQ ID NO: 247 sets forth the amino acid sequence of the eJM domain from the RLK protein encoded by gene locus LOC_Os07g36780 of Oryza sativa.

SEQ ID NO: 248 sets forth the amino acid sequence of the eJM domain from the RLK protein encoded by gene locus Medtr0280s0040 of Medicago truncatula.

SEQ ID NO: 249 sets forth the amino acid sequence of the eJM domain from the RLK protein encoded by gene locus Medtr8g030500 of Medicago truncatula.

SEQ ID NO: 250 sets forth the amino acid sequence of the eJM domain from the RLK protein encoded by gene locus LOC_Os05g11750 of Oryza sativa.

SEQ ID NO: 251 sets forth the amino acid sequence of the eJM domain from the RLK protein encoded by gene locus AT3G24550 of Arabidopsis thaliana.

SEQ ID NO: 252 sets forth the amino acid sequence of the eJM domain from the RLK protein encoded by gene locus Solyc10g051330.1 of Solanum lycopersicum.

SEQ ID NO: 253 sets forth the amino acid sequence of the eJM domain from the RLK protein encoded by gene locus Solyc06g051030.2 of Solanum lycopersicum.

SEQ ID NO: 254 sets forth the amino acid sequence of the eJM domain from the RLK protein encoded by gene locus Medtr3g071480 of Medicago truncatula.

SEQ ID NO: 255 sets forth the amino acid sequence of the eJM domain from the RLK protein encoded by gene locus AT4G02010 of Arabidopsis thaliana.

SEQ ID NO: 256 sets forth the amino acid sequence of the eJM domain from the RLK protein encoded by gene locus Medtr7g109670 of Medicago truncatula.

SEQ ID NO: 257 sets forth the amino acid sequence of the eJM domain from the RLK protein encoded by gene locus LOC_Os01g14932 of Oryza sativa.

SEQ ID NO: 258 sets forth the amino acid sequence of the eJM domain from the RLK protein encoded by gene locus LOC_Os05g12680 of Oryza sativa.

SEQ ID NO: 259 sets forth the amino acid sequence of the eJM domain from the RLK protein encoded by gene locus Solyc12g007110.1 of Solanum lycopersicum.

SEQ ID NO: 260 sets forth the amino acid sequence of the eJM domain from the RLK protein encoded by gene locus Solyc03g123740.2 of Solanum lycopersicum.

SEQ ID NO: 261 sets forth the amino acid sequence of the eJM domain from the RLK protein encoded by gene locus Medtr7g117520 of Medicago truncatula.

SEQ ID NO: 262 sets forth the amino acid sequence of the eJM domain from the RLK protein encoded by gene locus AT1G53730 of Arabidopsis thaliana.

SEQ ID NO: 263 sets forth the amino acid sequence of the eJM domain from the RLK protein encoded by gene locus LOC_Os03g08550 of Oryza sativa.

SEQ ID NO: 264 sets forth the amino acid sequence of the eJM domain from the RLK protein encoded by gene locus AT1G48480 of Arabidopsis thaliana.

SEQ ID NO: 265 sets forth the amino acid sequence of the eJM domain from the RLK protein encoded by gene locus Medtr8g107470 of Medicago truncatula.

SEQ ID NO: 266 sets forth the amino acid sequence of the eJM domain from the RLK protein encoded by gene locus AT3G28040 of Arabidopsis thaliana.

SEQ ID NO: 267 sets forth the amino acid sequence of the eJM domain from the RLK protein encoded by gene locus Medtr7g022160v2 of Medicago truncatula.

SEQ ID NO: 268 sets forth the amino acid sequence of the eJM domain from the RLK protein encoded by gene locus Solyc09g098290.2 of Solanum lycopersicum.

SEQ ID NO: 269 sets forth the amino acid sequence of the eJM domain from the RLK protein encoded by gene locus LOC_Os01g72700 of Oryza sativa.

SEQ ID NO: 270 sets forth the amino acid sequence of the eJM domain from the RLK protein encoded by gene locus AT1G66150 of Arabidopsis thaliana.

SEQ ID NO: 271 sets forth the amino acid sequence of the eJM domain from the RLK protein encoded by gene locus Medtr5g077430 of Medicago truncatula.

SEQ ID NO: 272 sets forth the amino acid sequence of the eJM domain from the RLK protein encoded by gene locus LOC_Os03g50810 of Oryza sativa.

SEQ ID NO: 273 sets forth the amino acid sequence of the eJM domain from the RLK protein encoded by gene locus AT5G56890 of Arabidopsis thaliana.

SEQ ID NO: 274 sets forth the amino acid sequence of the eJM domain from the RLK protein encoded by gene locus Medtr2g039290 of Medicago truncatula.

SEQ ID NO: 275 sets forth the amino acid sequence of the eJM domain from the RLK protein encoded by gene locus AT3G46420 of Arabidopsis thaliana.

SEQ ID NO: 276 sets forth the amino acid sequence of the eJM domain from the RLK protein encoded by gene locus Medtr8g015340 of Medicago truncatula.

SEQ ID NO: 277 sets forth the amino acid sequence of the eJM domain from the RLK protein encoded by gene locus LOC_Os05g44970 of Oryza sativa.

SEQ ID NO: 278 sets forth the amino acid sequence of the eJM domain from the RLK protein encoded by gene locus LOC_Os03g21540 of Oryza sativa.

SEQ ID NO: 279 sets forth the amino acid sequence of the eJM domain from the RLK protein encoded by gene locus Medtr7g073660 of Medicago truncatula.

SEQ ID NO: 280 sets forth the amino acid sequence of the eJM domain from the RLK protein encoded by gene locus Solyc01g108000.2 of Solanum lycopersicum.

SEQ ID NO: 281 sets forth the amino acid sequence of the eJM domain from the RLK protein encoded by gene locus Medtr4g085810 of Medicago truncatula.

SEQ ID NO: 282 sets forth the amino acid sequence of the eJM domain from the RLK protein encoded by gene locus AT1g49730 of Arabidopsis thaliana.

SEQ ID NO: 283 sets forth Solyc06g071810.1 of Solanum lycopersicum. the amino acid sequence of the eJM domain from the RLK protein encoded by gene locus

SEQ ID NO: 284 sets forth the amino acid sequence of the eJM domain from the RLK protein encoded by gene locus Medtr3g075440 of Medicago truncatula.

SEQ ID NO: 285 sets forth the amino acid sequence of the eJM domain from the RLK protein encoded by gene locus LOC_Os06g18000 of Oryza sativa.

SEQ ID NO: 286 sets forth the amino acid sequence of the eJM domain from the RLK protein encoded by gene locus AT1G18390 of Arabidopsis thaliana.

SEQ ID NO: 287 sets forth the amino acid sequence of the eJM domain from the RLK protein encoded by gene locus Medtr7g082470v2 of Medicago truncatula.

SEQ ID NO: 288 sets forth the amino acid sequence of the eJM domain from the RLK protein encoded by gene locus Solyc02g086210.2 of Solanum lycopersicum.

SEQ ID NO: 289 sets forth the amino acid sequence of the eJM domain from the RLK protein encoded by gene locus AT1G66930 of Arabidopsis thaliana.

SEQ ID NO: 290 sets forth the amino acid sequence of the eJM domain from the RLK protein encoded by gene locus Medtr1g064560 of Medicago truncatula.

SEQ ID NO: 291 sets forth the amino acid sequence of the eJM domain from the RLK protein encoded by gene locus AT2G39180 of Arabidopsis thaliana.

SEQ ID NO: 292 sets forth the amino acid sequence of the eJM domain from the RLK protein encoded by gene locus LOC_Os04g51040 of Oryza sativa.

SEQ ID NO: 293 sets forth the amino acid sequence of the eJM domain from the RLK protein encoded by gene locus LOC_Os04g51050 of Oryza sativa.

SEQ ID NO: 294 sets forth the amino acid sequence of the eJM domain from the RLK protein encoded by gene locus Solyc11g072140.1 of Solanum lycopersicum.

SEQ ID NO: 295 sets forth the amino acid sequence of the eJM domain from the RLK protein encoded by gene locus Solyc01g109650.2 of Solanum lycopersicum.

SEQ ID NO: 296 sets forth the amino acid sequence of the eJM domain from the RLK protein encoded by gene locus Medtr3g116640 of Medicago truncatula.

SEQ ID NO: 297 sets forth the amino acid sequence of the eJM domain from the RLK protein encoded by gene locus AT2G16250 of Arabidopsis thaliana.

SEQ ID NO: 298 sets forth the amino acid sequence of the eJM domain from the RLK protein encoded by gene locus AT4G00340 of Arabidopsis thaliana.

SEQ ID NO: 299 sets forth the amino acid sequence of the eJM domain from the RLK protein encoded by gene locus Solyc01g094830.2 of Solanum lycopersicum.

SEQ ID NO: 300 sets forth the amino acid sequence of the eJM domain from the RLK protein encoded by gene locus Medtr2g084120 of Medicago truncatula.

SEQ ID NO: 301 sets forth the amino acid sequence of the eJM domain from the RLK protein encoded by gene locus Solyc03g019980.1 of Solanum lycopersicum.

SEQ ID NO: 302 sets forth the amino acid sequence of the eJM domain from the RLK protein encoded by gene locus OsXa21LRR of Oryza sativa.

SEQ ID NO: 303 sets forth the amino acid sequence of the eJM domain from the RLK protein encoded by gene locus Medtr5g044680 of Medicago truncatula.

SEQ ID NO: 304 sets forth the amino acid sequence of the eJM domain from the RLK protein encoded by gene locus Medtr5g019070v2 of Medicago truncatula.

SEQ ID NO: 305 sets forth the amino acid sequence of the eJM domain from the RLK protein encoded by gene locus Solyc06g076910.1 of Solanum lycopersicum.

SEQ ID NO: 306 sets forth the amino acid sequence of the eJM domain from the RLK protein encoded by gene locus LOC_Os12g42520 of Oryza sativa.

SEQ ID NO: 307 sets forth the amino acid sequence of the eJM domain from the RLK protein encoded by gene locus AT3G47090 of Arabidopsis thaliana.

SEQ ID NO: 308 sets forth the amino acid sequence of the eJM domain from the RLK protein encoded by gene locus AT5G20480 of Arabidopsis thaliana. AT5G20480 is the locus that encodes AtEFR. The full-length AtEFR amino acid sequence is set forth in SEQ ID NO: 6.

SEQ ID NO: 309 sets forth the amino acid sequence of the eJM domain from the RLK protein encoded by gene locus Solyc04g051510.1 of Solanum lycopersicum.

SEQ ID NO: 310 sets forth the amino acid sequence of the eJM domain from the RLK protein encoded by gene locus Medtr3g095100 of Medicago truncatula.

SEQ ID NO: 311 sets forth the amino acid sequence of the eJM domain from the RLK protein encoded by gene locus AT4G39400 of Arabidopsis thaliana.

SEQ ID NO: 312 sets forth the amino acid sequence of the eJM domain from the RLK protein encoded by gene locus LOC_Os09g12240 of Oryza sativa.

SEQ ID NO: 313 sets forth the amino acid sequence of the eJM domain from the RLK protein encoded by gene locus AT2G26330 of Arabidopsis thaliana.

SEQ ID NO: 314 sets forth the amino acid sequence of the eJM domain from the RLK protein encoded by gene locus Solyc08g061560.2 of Solanum lycopersicum.

SEQ ID NO: 315 sets forth the amino acid sequence of the eJM domain from the RLK protein encoded by gene locus LOC_Os03g56270 of Oryza sativa.

SEQ ID NO: 316 sets forth the amino acid sequence of the eJM domain from the RLK protein encoded by gene locus Solyc04g081590.2 of Solanum lycopersicum.

SEQ ID NO: 317 sets forth the amino acid sequence of the eJM domain from the RLK protein encoded by gene locus AT1G75820 of Arabidopsis thaliana.

SEQ ID NO: 318 sets forth the amino acid sequence of the eJM domain from the RLK protein encoded by gene locus Medtr4g070970 of Medicago truncatula.

SEQ ID NO: 319 sets forth the amino acid sequence of the eJM domain from the RLK protein encoded by gene locus Solyc10g047140.1 of Solanum lycopersicum.

SEQ ID NO: 320 sets forth the amino acid sequence of the eJM domain from the RLK protein encoded by gene locus AT4G33430 of Arabidopsis thaliana.

SEQ ID NO: 321 sets forth the amino acid sequence of the eJM domain from the RLK protein encoded by gene locus Medtr2g008360 of Medicago truncatula.

SEQ ID NO: 322 sets forth the amino acid sequence of the eJM domain from the RLK protein encoded by gene locus LOC_Os08g07760 of Oryza sativa.

SEQ ID NO: 323 sets forth the amino acid sequence of the eJM domain from the RLK protein encoded by gene locus LOC_Os05g40770 of Oryza sativa.

SEQ ID NO: 324 sets forth the amino acid sequence of the eJM domain from the RLK protein encoded by gene locus Medtr8g070880 of Medicago truncatula.

SEQ ID NO: 325 sets forth the amino acid sequence of the eJM domain from the RLK protein encoded by gene locus LOC_Os12g10740 of Oryza sativa.

SEQ ID NO: 326 sets forth the amino acid sequence of the eJM domain from the RLK protein encoded by gene locus LOC_Os01g72990 of Oryza sativa.

SEQ ID NO: 327 sets forth the amino acid sequence of the eJM domain from the RLK protein encoded by gene locus Medtr3g062590 of Medicago truncatula.

SEQ ID NO: 328 sets forth the amino acid sequence of the eJM domain from the RLK protein encoded by gene locus AT3G21630 of Arabidopsis thaliana.

SEQ ID NO: 329 sets forth the amino acid sequence of the eJM domain from the RLK protein encoded by gene locus Medtr3g080050 of Medicago truncatula.

SEQ ID NO: 330 sets forth the amino acid sequence of the eJM domain from the RLK protein encoded by gene locus Solyc07g049180.2 of Solanum lycopersicum.

SEQ ID NO: 331 sets forth the amino acid sequence of the eJM domain from the RLK protein encoded by gene locus LOC_Os08g42580 of Oryza sativa.

SEQ ID NO: 332 sets forth the amino acid sequence of the eJM domain from the RLK protein encoded by gene locus Solyc12g014350.1 of Solanum lycopersicum.

SEQ ID NO: 333 sets forth the amino acid sequence of the eJM domain from the RLK protein encoded by gene locus Medtr2g075010 of Medicago truncatula.

SEQ ID NO: 334 sets forth the amino acid sequence of the eJM domain from the RLK protein encoded by gene locus AT3G14840 of Arabidopsis thaliana.

SEQ ID NO: 335 sets forth the amino acid sequence of the eJM domain from the RLK protein encoded by gene locus LOC_Os09g17630 of Oryza sativa.

SEQ ID NO: 336 sets forth the amino acid sequence of the eJM domain from the RLK protein encoded by gene locus Medtr7g092050 of Medicago truncatula.

SEQ ID NO: 337 sets forth the amino acid sequence of the eJM domain from the RLK protein encoded by gene locus AT2G41890 of Arabidopsis thaliana.

SEQ ID NO: 338 sets forth the amino acid sequence of the eJM domain from the RLK protein encoded by gene locus Solyc03g063650.1 of Solanum lycopersicum.

SEQ ID NO: 339 sets forth the amino acid sequence of the eJM domain from the RLK protein encoded by gene locus AT1G11050 of Arabidopsis thaliana.

SEQ ID NO: 340 sets forth the amino acid sequence of the eJM domain from the RLK protein encoded by gene locus Medtr2g006910 of Medicago truncatula.

SEQ ID NO: 341 sets forth the amino acid sequence of the eJM domain from the RLK protein encoded by gene locus LOC_Os05g34950 of Oryza sativa.

SEQ ID NO: 342 sets forth the amino acid sequence of the eJM domain from the RLK protein encoded by gene locus Medtr2g043380 of Medicago truncatula.

SEQ ID NO: 343 sets forth the amino acid sequence of the eJM domain from the RLK protein encoded by gene locus Solyc02g068370.2 of Solanum lycopersicum.

SEQ ID NO: 344 sets forth the amino acid sequence of the eJM domain from the RLK protein encoded by gene locus Solyc07g055180.2 of Solanum lycopersicum.

SEQ ID NO: 345 sets forth the amino acid sequence of the eJM domain from the RLK protein encoded by gene locus Solyc05g053010.1 of Solanum lycopersicum.

SEQ ID NO: 346 sets forth the amino acid sequence of the eJM domain from the RLK protein encoded by gene locus Solyc03g118510.2 of Solanum lycopersicum.

SEQ ID NO: 347 sets forth the amino acid sequence of the eJM domain from the RLK protein encoded by gene locus LOC_Os01g51290 of Oryza sativa.

SEQ ID NO: 348 sets forth the amino acid sequence of the eJM domain from the RLK protein encoded by gene locus LOC_Os05g33080 of Oryza sativa.

SEQ ID NO: 349 sets forth the amino acid sequence of the eJM domain from the RLK protein encoded by gene locus Solyc11g006040.1 of Solanum lycopersicum.

SEQ ID NO: 350 sets forth the amino acid sequence of the eJM domain from the RLK protein encoded by gene locus AT1G52310 of Arabidopsis thaliana.

SEQ ID NO: 351 sets forth the amino acid sequence of the eJM domain from the RLK protein encoded by gene locus LOC_Os03g51440 of Oryza sativa.

SEQ ID NO: 352 sets forth the amino acid sequence of the eJM domain from the RLK protein encoded by gene locus Solyc09g007750.2 of Solanum lycopersicum.

SEQ ID NO: 353 sets forth the amino acid sequence of the eJM domain from the RLK protein encoded by gene locus Solyc03g119240.2 of Solanum lycopersicum.

SEQ ID NO: 354 sets forth the amino acid sequence of the eJM domain from the RLK protein encoded by gene locus Medtr6g088610 of Medicago truncatula.

SEQ ID NO: 355 sets forth the amino acid sequence of the eJM domain from the RLK protein encoded by gene locus AT2G40270 of Arabidopsis thaliana.

SEQ ID NO: 356 sets forth the amino acid sequence of the eJM domain from the RLK protein encoded by gene locus LOC_Os04g59320 of Oryza sativa.

SEQ ID NO: 357 sets forth the amino acid sequence of the eJM domain from the RLK protein encoded by gene locus LOC_Os03g50450 of Oryza sativa.

SEQ ID NO: 358 sets forth the amino acid sequence of the eJM domain from the RLK protein encoded by gene locus LOC_Os01g01410 of Oryza sativa.

SEQ ID NO: 359 sets forth the amino acid sequence of the eJM domain from the RLK protein encoded by gene locus Solyc04g039730.2 of Solanum lycopersicum.

SEQ ID NO: 360 sets forth the amino acid sequence of the eJM domain from the RLK protein encoded by gene locus Solyc02g080040.2 of Solanum lycopersicum.

SEQ ID NO: 361 sets forth the amino acid sequence of the eJM domain from the RLK protein encoded by gene locus Solyc01g079340.2 of Solanum lycopersicum.

SEQ ID NO: 362 sets forth the amino acid sequence of the eJM domain from the RLK protein encoded by gene locus LOC_Os03g16260 of Oryza sativa.

SEQ ID NO: 363 sets forth the amino acid sequence of the eJM domain from the RLK protein encoded by gene locus AT3g59350 of Arabidopsis thaliana.

SEQ ID NO: 364 sets forth the amino acid sequence of the eJM domain from the RLK protein (WAK1) encoded by gene locus AT1G21250 of Arabidopsis thaliana.

SEQ ID NO: 365 sets forth the amino acid sequence of the eJM domain from the RLK protein encoded by gene locus Solyc09g015240.1 of Solanum lycopersicum.

SEQ ID NO: 366 sets forth the amino acid sequence of the eJM domain from the RLK protein encoded by gene locus AT5G07150 of Arabidopsis thaliana.

SEQ ID NO: 367 sets forth the amino acid sequence of the eJM domain from the RLK protein encoded by gene locus AT2G31880 of Arabidopsis thaliana. AT2G31880 is the locus that encodes AtSOBIR1. The full-length AtSOBIR1 amino acid sequence is set forth in SEQ ID NO: 8.

SEQ ID NO: 368 sets forth the amino acid sequence of the eJM domain from the RLK protein encoded by gene locus LOC_Os01g04409 of Oryza sativa.

SEQ ID NO: 369 sets forth the amino acid sequence of the eJM domain from the RLK protein encoded by gene locus LOC_Os01g49614 of Oryza sativa.

SEQ ID NO: 370 sets forth the amino acid sequence of the eJM domain from the RLK protein encoded by gene locus AT3G51550 of Arabidopsis thaliana.

SEQ ID NO: 371 sets forth the amino acid sequence of the eJM domain from the RLK protein encoded by gene locus Solyc01g104050.2 of Solanum lycopersicum.

SEQ ID NO: 372 sets forth the amino acid sequence of the eJM domain from the RLK protein encoded by gene locus LOC_Os08g01830 of Oryza sativa.

SEQ ID NO: 373 sets forth the amino acid sequence of the eJM domain from the RLK protein encoded by gene locus AT1G66980 of Arabidopsis thaliana.

SEQ ID NO: 374 sets forth the amino acid sequence of the eJM domain from the RLK protein encoded by gene locus AT5G38210 of Arabidopsis thaliana.

SEQ ID NO: 375 sets forth the amino acid sequence of the eJM domain from the RLK protein encoded by gene locus Medtr1g105650 of Medicago truncatula.

SEQ ID NO: 376 sets forth the amino acid sequence of the eJM domain from the RLK protein encoded by gene locus AT4G23190 of Arabidopsis thaliana.

SEQ ID NO: 377 sets forth the amino acid sequence of the eJM domain from the RLK protein encoded by gene locus LOC_Os07g35310 of Oryza sativa.

SEQ ID NO: 378 sets forth the amino acid sequence of the eJM domain from the RLK protein encoded by gene locus LOC_Os01g45520 of Oryza sativa.

SEQ ID NO: 379 sets forth the amino acid sequence of the eJM domain from the RLK protein encoded by gene locus Medtr1g082580 of Medicago truncatula.

SEQ ID NO: 380 sets forth the amino acid sequence of the eJM domain from the RLK protein encoded by gene locus AT2G45340 of Arabidopsis thaliana.

SEQ ID NO: 381 sets forth the amino acid sequence of the eJM domain from the RLK protein encoded by gene locus Solyc01g091230.2 of Solanum lycopersicum.

SEQ ID NO: 382 sets forth the amino acid sequence of the eJM domain from the RLK protein encoded by gene locus LOC_Os06g04370 of Oryza sativa.

SEQ ID NO: 383 sets forth the amino acid sequence of the eJM domain from the RLK protein encoded by gene locus Medtr6g060230 of Medicago truncatula.

SEQ ID NO: 384 sets forth the amino acid sequence of the eJM domain from the RLK protein encoded by gene locus Solyc03g121230.2 of Solanum lycopersicum.

SEQ ID NO: 385 sets forth the amino acid sequence of the eJM domain from the RLK protein encoded by gene locus Solyc02g079590.2 of Solanum lycopersicum.

SEQ ID NO: 386 sets forth the amino acid sequence of the eJM domain from the RLK protein encoded by gene locus AT1g80640 of Arabidopsis thaliana.

SEQ ID NO: 387 sets forth the amino acid sequence of the eJM domain from the RLK protein encoded by gene locus AT3G59700 of Arabidopsis thaliana.

SEQ ID NO: 388 sets forth the amino acid sequence of the eJM domain from the RLK protein encoded by gene locus LOC_Os03g18370 of Oryza sativa.

SEQ ID NO: 389 sets forth the amino acid sequence of the eJM domain from the RLK protein encoded by gene locus AT5G38280 of Arabidopsis thaliana.

SEQ ID NO: 390 sets forth the amino acid sequence of the eJM domain from the RLK protein encoded by gene locus Solyc07g039340.2 of Solanum lycopersicum.

Each of SEQ ID NOS: 391-540, 548-553, and 567-569 sets forth the amino acid sequence of transmembrane (TM) domain from a plant receptor-like kinases (RLK) from Arabidopsis thaliana, Medicago truncatula, Oryza sativa, or Solanum lycopersicum that can be used in the methods and compositions of present invention as described elsewhere herein. In the following descriptions for each of these sequences, the RLK subgroup (if assigned or otherwise known) and the plant species from which each sequence the RLK was derived is provided in parentheses.

SEQ ID NO: 391 sets forth the amino acid sequence of the TM domain from the RLK protein encoded by gene locus LOC_Os09g17630 of Oryza sativa.

SEQ ID NO: 392 sets forth the amino acid sequence of the TM domain from the RLK protein encoded by gene locus Solyc01g094830.2v2 of Solanum lycopersicum.

SEQ ID NO: 393 sets forth the amino acid sequence of the TM domain from the RLK protein encoded by gene locus AT2G26330 of Arabidopsis thaliana.

SEQ ID NO: 394 sets forth the amino acid sequence of the TM domain from the RLK protein encoded by gene locus Solyc08g061560.2 of Solanum lycopersicum.

SEQ ID NO: 395 sets forth the amino acid sequence of the TM domain from the RLK protein encoded by gene locus Medtr3g080050 of Medicago truncatula.

SEQ ID NO: 396 sets forth the amino acid sequence of the TM domain from the RLK protein encoded by gene locus Medtr7g062990 of Medicago truncatula.

SEQ ID NO: 397 sets forth the amino acid sequence of the TM domain from the RLK protein encoded by gene locus LOC_Os07g03830 of Oryza sativa.

SEQ ID NO: 398 sets forth the amino acid sequence of the TM domain from the RLK protein encoded by gene locus Solyc11g072140.1 of Solanum lycopersicum.

SEQ ID NO: 399 sets forth the amino acid sequence of the TM domain from the RLK protein encoded by gene locus LOC_Os04g51040 of Oryza sativa.

SEQ ID NO: 400 sets forth the amino acid sequence of the TM domain from the RLK protein encoded by gene locus Solyc04g039730.2 of Solanum lycopersicum.

SEQ ID NO: 401 sets forth the amino acid sequence of the TM domain from the RLK protein encoded by gene locus Medtr3g071480 of Medicago truncatula.

SEQ ID NO: 402 sets forth the amino acid sequence of the TM domain from the RLK protein encoded by gene locus AT2G40270 of Arabidopsis thaliana.

SEQ ID NO: 403 sets forth the amino acid sequence of the TM domain from the RLK protein encoded by gene locus Medtr4g091670 of Medicago truncatula.

SEQ ID NO: 404 sets forth the amino acid sequence of the TM domain from the RLK protein encoded by gene locus Solyc09g015240.1 of Solanum lycopersicum.

SEQ ID NO: 405 sets forth the amino acid sequence of the TM domain from the RLK protein encoded by gene locus LOC_Os03g50450 of Oryza sativa.

SEQ ID NO: 406 sets forth the amino acid sequence of the TM domain from the RLK protein encoded by gene locus Medtr8g107470 of Medicago truncatula.

SEQ ID NO: 407 sets forth the amino acid sequence of the TM domain from the RLK protein encoded by gene locus AT1G48480 of Arabidopsis thaliana.

SEQ ID NO: 408 sets forth the amino acid sequence of the TM domain from the RLK protein encoded by gene locus Solyc03g118510.2 of Solanum lycopersicum.

SEQ ID NO: 409 sets forth the amino acid sequence of the TM domain from the RLK protein encoded by gene locus AT3G21630 of Arabidopsis thaliana.

SEQ ID NO: 410 sets forth the amino acid sequence of the TM domain from the RLK protein encoded by gene locus LOC_Os01g04409 of Oryza sativa.

SEQ ID NO: 411 sets forth the amino acid sequence of the TM domain from the RLK protein encoded by gene locus AT1G65790 of Arabidopsis thaliana.

SEQ ID NO: 412 sets forth the amino acid sequence of the TM domain from the RLK protein encoded by gene locus Solyc10g051330.1 of Solanum lycopersicum.

SEQ ID NO: 413 sets forth the amino acid sequence of the TM domain from the RLK protein encoded by gene locus LOC_Os04g51050 of Oryza sativa.

SEQ ID NO: 414 sets forth the amino acid sequence of the TM domain from the RLK protein encoded by gene locus AT3G47090 of Arabidopsis thaliana.

SEQ ID NO: 415 sets forth the amino acid sequence of the TM domain from the RLK protein encoded by gene locus AT5G20480 of Arabidopsis thaliana. AT5G20480 is the locus that encodes AtEFR. The full-length AtEFR amino acid sequence is set forth in SEQ ID NO: 6.

SEQ ID NO: 416 sets forth the amino acid sequence of the TM domain from the RLK protein encoded by gene locus Medtr7g082470v2 of Medicago truncatula.

SEQ ID NO: 417 sets forth the amino acid sequence of the TM domain from the RLK protein encoded by gene locus Medtr6g088610 of Medicago truncatula.

SEQ ID NO: 418 sets forth the amino acid sequence of the TM domain from the RLK protein encoded by gene locus AT3G24550 of Arabidopsis thaliana.

SEQ ID NO: 419 sets forth the amino acid sequence of the TM domain from the RLK protein encoded by gene locus Solyc12g007110.1 of Solanum lycopersicum.

SEQ ID NO: 420 sets forth the amino acid sequence of the TM domain from the RLK protein encoded by gene locus LOC_Os05g12680 of Oryza sativa.

SEQ ID NO: 421 sets forth the amino acid sequence of the TM domain from the RLK protein encoded by gene locus LOC_Os07g36780 of Oryza sativa.

SEQ ID NO: 422 sets forth the amino acid sequence of the TM domain from the RLK protein encoded by gene locus LOC_Os03g18370 of Oryza sativa.

SEQ ID NO: 423 sets forth the amino acid sequence of the TM domain from the RLK protein encoded by gene locus Solyc01g079340.2 of Solanum lycopersicum.

SEQ ID NO: 424 sets forth the amino acid sequence of the TM domain from the RLK protein encoded by gene locus AT4G02010v2 of Arabidopsis thaliana.

SEQ ID NO: 425 sets forth the amino acid sequence of the TM domain from the RLK protein encoded by gene locus Medtr7g109670 of Medicago truncatula.

SEQ ID NO: 426 sets forth the amino acid sequence of the TM domain from the RLK protein encoded by gene locus LOC_Os01g14932 of Oryza sativa.

SEQ ID NO: 427 sets forth the amino acid sequence of the TM domain from the RLK protein encoded by gene locus Solyc06g051030.2 of Solanum lycopersicum.

SEQ ID NO: 428 sets forth the amino acid sequence of the TM domain from the RLK protein encoded by gene locus LOC_Os03g51440 of Oryza sativa.

SEQ ID NO: 429 sets forth the amino acid sequence of the TM domain from the RLK protein encoded by gene locus Solyc10g047140.1 of Solanum lycopersicum.

SEQ ID NO: 430 sets forth the amino acid sequence of the TM domain from the RLK protein encoded by gene locus AT4G33430.1 of Arabidopsis thaliana.

SEQ ID NO: 431 sets forth the amino acid sequence of the TM domain from the RLK protein encoded by gene locus Medtr2g008360 of Medicago truncatula.

SEQ ID NO: 432 sets forth the amino acid sequence of the TM domain from the RLK protein encoded by gene locus LOC_Os08g07760 of Oryza sativa.

SEQ ID NO: 433 sets forth the amino acid sequence of the TM domain from the RLK protein encoded by gene locus LOC_Os08g42580 of Oryza sativa.

SEQ ID NO: 434 sets forth the amino acid sequence of the TM domain from the RLK protein encoded by gene locus LOC_Os05g40770 of Oryza sativa.

SEQ ID NO: 435 sets forth the amino acid sequence of the TM domain from the RLK protein encoded by gene locus AT3G51550 of Arabidopsis thaliana.

SEQ ID NO: 436 sets forth the amino acid sequence of the TM domain from the RLK protein encoded by gene locus LOC_Os03g21540 of Oryza sativa.

SEQ ID NO: 437 sets forth the amino acid sequence of the TM domain from the RLK protein encoded by gene locus Solyc07g049180.2 of Solanum lycopersicum.

SEQ ID NO: 438 sets forth the amino acid sequence of the TM domain from the RLK protein encoded by gene locus Medtr7g073660 of Medicago truncatula.

SEQ ID NO: 439 sets forth the amino acid sequence of the TM domain from the RLK protein encoded by gene locus LOC_Os03g50810 of Oryza sativa.

SEQ ID NO: 440 sets forth the amino acid sequence of the TM domain from the RLK protein encoded by gene locus Solyc11g006040.1 of Solanum lycopersicum.

SEQ ID NO: 441 sets forth the amino acid sequence of the TM domain from the RLK protein encoded by gene locus Medtr5g077430 of Medicago truncatula.

SEQ ID NO: 442 sets forth the amino acid sequence of the TM domain from the RLK protein encoded by gene locus AT1G66150 of Arabidopsis thaliana.

SEQ ID NO: 443 sets forth the amino acid sequence of the TM domain from the RLK protein encoded by gene locus Medtr3g062590 of Medicago truncatula.

SEQ ID NO: 444 sets forth the amino acid sequence of the TM domain from the RLK protein encoded by gene locus LOC_Os01g72990 of Oryza sativa.

SEQ ID NO: 445 sets forth the amino acid sequence of the TM domain from the RLK protein encoded by gene locus Medtr4g070970 of Medicago truncatula.

SEQ ID NO: 446 sets forth the amino acid sequence of the TM domain from the RLK protein encoded by gene locus LOC_Os08g01830 of Oryza sativa.

SEQ ID NO: 447 sets forth the amino acid sequence of the TM domain from the RLK protein encoded by gene locus LOC_Os03g16260 of Oryza sativa.

SEQ ID NO: 448 sets forth the amino acid sequence of the TM domain from the RLK protein encoded by gene locus AT1G66980 of Arabidopsis thaliana.

SEQ ID NO: 449 sets forth the amino acid sequence of the TM domain from the RLK protein encoded by gene locus LOC_Os12g10740 of Oryza sativa.

SEQ ID NO: 450 sets forth the amino acid sequence of the TM domain from the RLK protein encoded by gene locus Medtr2g075010 of Medicago truncatula.

SEQ ID NO: 451 sets forth the amino acid sequence of the TM domain from the RLK protein encoded by gene locus Solyc12g014350.1 of Solanum lycopersicum.

SEQ ID NO: 452 sets forth the amino acid sequence of the TM domain from the RLK protein encoded by gene locus AT3G14840 of Arabidopsis thaliana.

SEQ ID NO: 453 sets forth the amino acid sequence of the TM domain from the RLK protein encoded by gene locus Medtr0280s0040 of Medicago truncatula.

SEQ ID NO: 454 sets forth the amino acid sequence of the TM domain from the RLK protein encoded by gene locus Solyc03g123740.2 of Solanum lycopersicum.

SEQ ID NO: 455 sets forth the amino acid sequence of the TM domain from the RLK protein encoded by gene locus LOC_Os03g08550 of Oryza sativa.

SEQ ID NO: 456 sets forth the amino acid sequence of the TM domain from the RLK protein encoded by gene locus Medtr7g117520 of Medicago truncatula.

SEQ ID NO: 457 sets forth the amino acid sequence of the TM domain from the RLK protein encoded by gene locus AT1G53730 of Arabidopsis thaliana.

SEQ ID NO: 458 sets forth the amino acid sequence of the TM domain from the RLK protein encoded by gene locus AT5G07150 of Arabidopsis thaliana.

SEQ ID NO: 459 sets forth the amino acid sequence of the TM domain from the RLK protein encoded by gene locus Solyc01g104050.2 of Solanum lycopersicum.

SEQ ID NO: 460 sets forth the amino acid sequence of the TM domain from the RLK protein encoded by gene locus Medtr2g006910 of Medicago truncatula.

SEQ ID NO: 461 sets forth the amino acid sequence of the TM domain from the RLK protein encoded by gene locus AT1G11050 of Arabidopsis thaliana.

SEQ ID NO: 462 sets forth the amino acid sequence of the TM domain from the RLK protein encoded by gene locus Solyc03g119240.2 of Solanum lycopersicum.

SEQ ID NO: 463 sets forth the amino acid sequence of the TM domain from the RLK protein encoded by gene locus Medtr3g075440 of Medicago truncatula.

SEQ ID NO: 464 sets forth the amino acid sequence of the TM domain from the RLK protein encoded by gene locus AT2G31880 of Arabidopsis thaliana. AT2G31880 is the locus that encodes AtSOBIR1. The full-length AtSOBIR1 amino acid sequence is set forth in SEQ ID NO: 8.

SEQ ID NO: 465 sets forth the amino acid sequence of the TM domain from the RLK protein encoded by gene locus LOC_Os06g1800 of Oryza sativa.

SEQ ID NO: 466 sets forth the amino acid sequence of the TM domain from the RLK protein encoded by gene locus Solyc06g071810.1 of Solanum lycopersicum.

SEQ ID NO: 467 sets forth the amino acid sequence of the TM domain from the RLK protein encoded by gene locus AT3G59700 of Arabidopsis thaliana.

SEQ ID NO: 468 sets forth the amino acid sequence of the TM domain from the RLK protein encoded by gene locus AT1G18390v2 of Arabidopsis thaliana.

SEQ ID NO: 469 sets forth the amino acid sequence of the TM domain from the RLK protein encoded by gene locus AT5G38210v2 of Arabidopsis thaliana.

SEQ ID NO: 470 sets forth the amino acid sequence of the TM domain from the RLK protein encoded by gene locus Medtr8g070880 of Medicago truncatula.

SEQ ID NO: 471 sets forth the amino acid sequence of the TM domain from the RLK protein encoded by gene locus Medtr7g092050 of Medicago truncatula.

SEQ ID NO: 472 sets forth the amino acid sequence of the TM domain from the RLK protein encoded by gene locus AT2G41890 of Arabidopsis thaliana.

SEQ ID NO: 473 sets forth the amino acid sequence of the TM domain from the RLK protein encoded by gene locus Medtr1g105650 of Medicago truncatula.

SEQ ID NO: 474 sets forth the amino acid sequence of the TM domain from the RLK protein encoded by gene locus LOC_Os03g35600 of Oryza sativa.

SEQ ID NO: 475 sets forth the amino acid sequence of the TM domain from the RLK protein encoded by gene locus Solyc03g121230.2 of Solanum lycopersicum.

SEQ ID NO: 476 sets forth the amino acid sequence of the TM domain from the RLK protein encoded by gene locus Medtr5g044680 of Medicago truncatula.

SEQ ID NO: 477 sets forth the amino acid sequence of the TM domain from the RLK protein encoded by gene locus Medtr5g019070v2 of Medicago truncatula.

SEQ ID NO: 478 sets forth the amino acid sequence of the TM domain from the RLK protein encoded by gene locus Solyc02g080040.2 of Solanum lycopersicum.

SEQ ID NO: 479 sets forth the amino acid sequence of the TM domain from the RLK protein encoded by gene locus AT4G23190 of Arabidopsis thaliana.

SEQ ID NO: 480 sets forth the amino acid sequence of the TM domain from the RLK protein encoded by gene locus Solyc07g055180.2 of Solanum lycopersicum.

SEQ ID NO: 481 sets forth the amino acid sequence of the TM domain from the RLK protein encoded by gene locus Medtr2g084120 of Medicago truncatula.

SEQ ID NO: 482 sets forth the amino acid sequence of the TM domain from the RLK protein encoded by gene locus LOC_Os05g11750 of Oryza sativa.

SEQ ID NO: 483 sets forth the amino acid sequence of the TM domain from the RLK protein encoded by gene locus AT1G66930v2 of Arabidopsis thaliana.

SEQ ID NO: 484 sets forth the amino acid sequence of the TM domain from the RLK protein encoded by gene locus LOC_Os05g34950 of Oryza sativa.

SEQ ID NO: 485 sets forth the amino acid sequence of the TM domain from the RLK protein encoded by gene locus AT3g59350 of Arabidopsis thaliana.

SEQ ID NO: 486 sets forth the amino acid sequence of the TM domain from the RLK protein encoded by gene locus AT3G08870 of Arabidopsis thaliana.

SEQ ID NO: 487 sets forth the amino acid sequence of the TM domain from the RLK protein encoded by gene locus Medtr8g015340 of Medicago truncatula.

SEQ ID NO: 488 sets forth the amino acid sequence of the TM domain from the RLK protein encoded by gene locus LOC_Os12g42520 of Oryza sativa.

SEQ ID NO: 489 sets forth the amino acid sequence of the TM domain from the RLK protein encoded by gene locus Solyc03g019980.1v2 of Solanum lycopersicum.

SEQ ID NO: 490 sets forth the amino acid sequence of the TM domain from the RLK protein encoded by gene locus LOC_Os09g12240 of Oryza sativa.

SEQ ID NO: 491 sets forth the amino acid sequence of the TM domain from the RLK protein encoded by gene locus OsXa21 of Oryza sativa.

SEQ ID NO: 492 sets forth the amino acid sequence of the TM domain from the RLK protein encoded by gene locus Medtr1g064560 of Medicago truncatula.

SEQ ID NO: 493 sets forth the amino acid sequence of the TM domain from the RLK protein encoded by gene locus Solyc06g076910.1 of Solanum lycopersicum.

SEQ ID NO: 494 sets forth the amino acid sequence of the TM domain from the RLK protein encoded by gene locus LOC_Os07g35310 of Oryza sativa.

SEQ ID NO: 495 sets forth the amino acid sequence of the TM domain from the RLK protein encoded by gene locus AT1G75820 of Arabidopsis thaliana.

SEQ ID NO: 496 sets forth the amino acid sequence of the TM domain from the RLK protein encoded by gene locus AT5G38280 of Arabidopsis thaliana.

SEQ ID NO: 497 sets forth the amino acid sequence of the TM domain from the RLK protein encoded by gene locus Medtr4g085810 of Medicago truncatula.

SEQ ID NO: 498 sets forth the amino acid sequence of the TM domain from the RLK protein encoded by gene locus AT1g49730 of Arabidopsis thaliana.

SEQ ID NO: 499 sets forth the amino acid sequence of the TM domain from the RLK protein encoded by gene locus Solyc01g108000.2 of Solanum lycopersicum.

SEQ ID NO: 500 sets forth the amino acid sequence of the TM domain from the RLK protein encoded by gene locus LOC_Os04g59320 of Oryza sativa.

SEQ ID NO: 501 sets forth the amino acid sequence of the TM domain from the RLK protein encoded by gene locus AT3G46420 of Arabidopsis thaliana.

SEQ ID NO: 502 sets forth the amino acid sequence of the TM domain from the RLK protein encoded by gene locus LOC_Os01g45520 of Oryza sativa.

SEQ ID NO: 503 sets forth the amino acid sequence of the TM domain from the RLK protein encoded by gene locus Medtr2g043380 of Medicago truncatula.

SEQ ID NO: 504 sets forth the amino acid sequence of the TM domain from the RLK protein encoded by gene locus Solyc02g068370.2 of Solanum lycopersicum.

SEQ ID NO: 505 sets forth the amino acid sequence of the TM domain from the RLK protein encoded by gene locus LOC_Os01g01410 of Oryza sativa.

SEQ ID NO: 506 sets forth the amino acid sequence of the TM domain from the RLK protein encoded by gene locus AT1G52310 of Arabidopsis thaliana.

SEQ ID NO: 507 sets forth the amino acid sequence of the TM domain from the RLK protein encoded by gene locus AT4G00340 of Arabidopsis thaliana.

SEQ ID NO: 508 sets forth the amino acid sequence of the TM domain from the RLK protein encoded by gene locus Medtr3g116640 of Medicago truncatula.

SEQ ID NO: 509 sets forth the amino acid sequence of the TM domain from the RLK protein encoded by gene locus Solyc01g109650.2 of Solanum lycopersicum.

SEQ ID NO: 510 sets forth the amino acid sequence of the TM domain from the RLK protein encoded by gene locus AT2G16250 of Arabidopsis thaliana.

SEQ ID NO: 511 sets forth the amino acid sequence of the TM domain from the RLK protein encoded by gene locus AT1g80640 of Arabidopsis thaliana.

SEQ ID NO: 512 sets forth the amino acid sequence of the TM domain from the RLK protein (WAK1) encoded by gene locus AT1G21250 of Arabidopsis thaliana.

SEQ ID NO: 513 sets forth the amino acid sequence of the TM domain from the RLK protein encoded by gene locus Solyc04g051510.1 of Solanum lycopersicum.

SEQ ID NO: 514 sets forth the amino acid sequence of the TM domain from the RLK protein encoded by gene locus Medtr3g095100 of Medicago truncatula.

SEQ ID NO: 515 sets forth the amino acid sequence of the TM domain from the RLK protein encoded by gene locus AT4G39400 of Arabidopsis thaliana.

SEQ ID NO: 516 sets forth the amino acid sequence of the TM domain from the RLK protein encoded by gene locus Solyc09g007750.2 of Solanum lycopersicum.

SEQ ID NO: 517 sets forth the amino acid sequence of the TM domain from the RLK protein encoded by gene locus AT2G39180 of Arabidopsis thaliana.

SEQ ID NO: 518 sets forth the amino acid sequence of the TM domain from the RLK protein encoded by gene locus LOC_Os01g49614 of Oryza sativa.

SEQ ID NO: 519 sets forth the amino acid sequence of the TM domain from the RLK protein encoded by gene locus Solyc05g053010.1 of Solanum lycopersicum.

SEQ ID NO: 520 sets forth the amino acid sequence of the TM domain from the RLK protein encoded by gene locus ospi03g56270 of Oryza sativa.

SEQ ID NO: 521 sets forth the amino acid sequence of the TM domain from the RLK protein encoded by gene locus Solyc01g091230.2 of Solanum lycopersicum.

SEQ ID NO: 522 sets forth the amino acid sequence of the TM domain from the RLK protein encoded by gene locus AT2G45340 of Arabidopsis thaliana.

SEQ ID NO: 523 sets forth the amino acid sequence of the TM domain from the RLK protein encoded by gene locus Medtr8g030500 of Medicago truncatula.

SEQ ID NO: 524 sets forth the amino acid sequence of the TM domain from the RLK protein encoded by gene locus LOC_Os06g04370 of Oryza sativa.

SEQ ID NO: 525 sets forth the amino acid sequence of the TM domain from the RLK protein encoded by gene locus Medtr2g039290 of Medicago truncatula.

SEQ ID NO: 526 sets forth the amino acid sequence of the TM domain from the RLK protein encoded by gene locus LOC_Os01g51290 of Oryza sativa.

SEQ ID NO: 527 sets forth the amino acid sequence of the TM domain from the RLK protein encoded by gene locus AT5G56890 of Arabidopsis thaliana.

SEQ ID NO: 528 sets forth the amino acid sequence of the TM domain from the RLK protein encoded by gene locus Solyc07g039340.2 of Solanum lycopersicum.

SEQ ID NO: 529 sets forth the amino acid sequence of the TM domain from the RLK protein encoded by gene locus Solyc09g098290.2 of Solanum lycopersicum.

SEQ ID NO: 530 sets forth the amino acid sequence of the TM domain from the RLK protein encoded by gene locus AT3G28040 of Arabidopsis thaliana.

SEQ ID NO: 531 sets forth the amino acid sequence of the TM domain from the RLK protein encoded by gene locus LOC_Os01g72700 of Oryza sativa.

SEQ ID NO: 532 sets forth the amino acid sequence of the TM domain from the RLK protein encoded by gene locus Medtr7g022160v2 of Medicago truncatula.

SEQ ID NO: 533 sets forth the amino acid sequence of the TM domain from the RLK protein encoded by gene locus Solyc02g086210.2 of Solanum lycopersicum.

SEQ ID NO: 534 sets forth the amino acid sequence of the TM domain from the RLK protein encoded by gene locus Medtr6g060230 of Medicago truncatula.

SEQ ID NO: 535 sets forth the amino acid sequence of the TM domain from the RLK protein encoded by gene locus LOC_Os05g44970 of Oryza sativa.

SEQ ID NO: 536 sets forth the amino acid sequence of the TM domain from the RLK protein encoded by gene locus Solyc04g081590.2 of Solanum lycopersicum.

SEQ ID NO: 537 sets forth the amino acid sequence of the TM domain from the RLK protein encoded by gene locus Medtr1g082580 of Medicago truncatula.

SEQ ID NO: 538 sets forth the amino acid sequence of the TM domain from the RLK protein encoded by gene locus Solyc03g063650.1 of Solanum lycopersicum.

SEQ ID NO: 539 sets forth the amino acid sequence of the TM domain from the RLK protein encoded by gene locus LOC_Os05g33080 of Oryza sativa.

SEQ ID NO: 540 sets forth the amino acid sequence of the TM domain from the RLK protein encoded by gene locus Solyc02g079590.2 of Solanum lycopersicum.

SEQ ID NO: 541 sets forth the amino acid sequence of the synthetic eJM domain, referred to herein as eJM(EEEE/ADQ−).

SEQ ID NO: 542 sets forth the amino acid sequence of the eJM domain from AtRLP1 of Arabidopsis thaliana.

SEQ ID NO: 543 sets forth the amino acid sequence of the eJM domain from AtRLP23 of Arabidopsis thaliana.

SEQ ID NO: 544 sets forth the amino acid sequence of the eJM domain from AtRLP30 of Arabidopsis thaliana.

SEQ ID NO: 545 sets forth the amino acid sequence of the eJM domain from AtRLP42 of Arabidopsis thaliana.

SEQ ID NO: 546 sets forth the amino acid sequence of the eJM domain from Cf-4 of Solanum lycopersicum.

SEQ ID NO: 547 sets forth the amino acid sequence of the eJM domain from Ve1 of Solanum lycopersicum.

SEQ ID NO: 548 sets forth the amino acid sequence of the TM domain from AtRLP1 of Arabidopsis thaliana.

SEQ ID NO: 549 sets forth the amino acid sequence of the TM domain from AtRLP23 of Arabidopsis thaliana.

SEQ ID NO: 550 sets forth the amino acid sequence of the TM domain from AtRLP30 of Arabidopsis thaliana.

SEQ ID NO: 551 sets forth the amino acid sequence of the TM domain from AtRLP42 of Arabidopsis thaliana.

SEQ ID NO: 552 sets forth the amino acid sequence of the TM domain from Cf-4 of Solanum lycopersicum.

SEQ ID NO: 553 sets forth the amino acid sequence of the TM domain from Ve1 of Solanum lycopersicum.

SEQ ID NO: 554 sets forth the amino acid sequence of the SP domain from AtRLP1 of Arabidopsis thaliana.

SEQ ID NO: 555 sets forth the amino acid sequence of the SP domain from AtRLP23 of Arabidopsis thaliana.

SEQ ID NO: 556 sets forth the amino acid sequence of the SP domain from AtRLP30 of Arabidopsis thaliana.

SEQ ID NO: 557 sets forth the amino acid sequence of the SP domain from AtRLP42 of Arabidopsis thaliana.

SEQ ID NO: 558 sets forth the amino acid sequence of the SP domain from Cf-4 of Solanum lycopersicum.

SEQ ID NO: 559 sets forth the amino acid sequence of the SP domain from Ve1 of Solanum lycopersicum.

SEQ ID NO: 560 sets forth an amino acid sequence comprising the amino acid sequence of the LRR domain from AtRLP23 of Arabidopsis thaliana.

SEQ ID NO: 561 sets forth the amino acid sequence of the kinase domain from the RLK protein (OsPi-d2) encoded by gene locus LOC_Os06g29810v2 (Oryza sativa).

SEQ ID NO: 562 sets forth the amino acid sequence of the kinase domain from the RLK protein (AtPEPR1) encoded by gene locus AT1G73080 (Arabidopsis thaliana).

SEQ ID NO: 563 sets forth the amino acid sequence of the kinase domain from the RLK protein (AtLYK5) encoded by gene locus AT2G33580 (Arabidopsis thaliana).

SEQ ID NO: 564 sets forth the amino acid sequence of the eJM domain from the RLK protein (OsPi-d2) encoded by gene locus LOC_Os06g29810v2 (Oryza sativa).

SEQ ID NO: 565 sets forth the amino acid sequence of the eJM domain from the RLK protein (AtPEPR1) encoded by gene locus AT1G73080 (Arabidopsis thaliana).

SEQ ID NO: 566 sets forth the amino acid sequence of the eJM domain from the RLK protein (AtLYK5) encoded by gene locus AT2G33580 (Arabidopsis thaliana).

SEQ ID NO: 567 sets forth the amino acid sequence of the TM domain from the RLK protein (OsPi-d2) encoded by gene locus LOC_Os06g29810v2 (Oryza sativa).

SEQ ID NO: 568 sets forth the amino acid sequence of the TM domain from the RLK protein (AtPEPR1) encoded by gene locus AT1G73080 (Arabidopsis thaliana).

SEQ ID NO: 569 sets forth the amino acid sequence of the TM domain from the RLK protein (AtLYK5) encoded by gene locus AT2G33580 (Arabidopsis thaliana).

SEQ ID NO: 570 sets forth the nucleotide sequence of the AtRLP23-AtPEPR1 polynucleotide construct. The construct comprises a first nucleotide sequence encoding the apoplastic and eJM domains of AtRLP23 (nucleotides 1-2550), operably linked to a second nucleotide sequence comprising a coding sequence for the AtPEPR1 TM domain and kinase domain (nucleotides 2551-3612). If desired, a stop codon (e.g. TAA, TAG, or TGA) can be operably linked to the 3′ end of a nucleic acid molecule comprising SEQ ID NO: 570.

SEQ ID NO: 571 sets forth the amino acid sequence of AtRLP23-AtPEPR1 protein encoded by SEQ ID NO: 19. The AtRLP23-AtPEPR1 protein comprises a first polypeptide comprising the AtRLP23 apoplastic and eJM domains (amino acids 1-850), operably linked to a second polypeptide comprising the OsXA21 TM and cytoplasmic domains (amino acids 851-1204).

SEQ ID NO: 572 sets forth the nucleotide sequence of the AtRLP23-AtPEPR1-3×FLAG polynucleotide construct. The construct comprises a first nucleotide sequence encoding the apoplastic and eJM domains of AtRLP23 (nucleotides 1-2550), operably linked to a second nucleotide sequence comprising a coding sequence for the AtPEPR1 TM domain and kinase domain (nucleotides 2551-3612), operably linked to a third nucleotide sequence encoding the 3×FLAG peptide (nucleotides 3613-3696). If desired, a stop codon (e.g. TAA, TAG, or TGA) can be operably linked to the 3′ end of a nucleic acid molecule comprising SEQ ID NO: 572. It is noted that in the Examples below, the stop codon TGA was used with this construct.

SEQ ID NO: 573 sets forth the amino acid sequence of AtRLP23-AtPEPR1-3×FLAG protein encoded by SEQ ID NO: 21. The AtRLP23-AtPEPR1-3×FLAG protein comprises a first polypeptide comprising the AtRLP23 apoplastic and eJM domains (amino acids 1-850), operably linked to a second polypeptide comprising the AtPEPR1 TM and kinase domains (amino acids 851-1204), operably linked to the 3×FLAG peptide (amino acids 1205-1232).

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention relates to methods for making and using engineered AtRLP23 proteins and nucleic acid molecules encoding such AtRLP23 proteins. Thus, the AtRLP23 proteins and nucleic acid molecules of the present invention are synthetic or artificial (i.e. non-naturally occurring) proteins and nucleic acid molecules. Such synthetic or artificial AtRLP23 proteins of the present invention are also referred to herein as “engineered AtRLP23 proteins”. The AtRLP23 proteins and nucleic acid molecules encoding them find use in enhancing the resistance of plants, particularly crop plants, to plant pathogens. Such crop plants with enhanced resistance to plant pathogens find use in agriculture by limiting or reducing plant disease.

The present invention further relates to compositions comprising at least one engineered AtRLP23 protein of the present invention and/or at least one nucleic acid molecule encoding such an engineered AtRLP23 protein. Such compositions include, but not limited to, plants, plant cells, and other host cells comprising one or more of such engineered AtRLP23 proteins and/or nucleic acid molecules, and expression cassettes and vectors comprising one or more of such nucleic acid molecules.

The present invention provides methods for making engineered AtRLP23 proteins comprising a leucine-rich-repeat (LRR) domain derived from a receptor-like protein (RLP) that is capable of recognizing in a plant a pathogen-associated molecular pattern (PAMP) derived from a necrosis- and ethylene-inducing protein 1 (Nep1)-like protein family of PAMPs. Nep1-like proteins (NLPs) are known to occur in bacteria, fungi, and oomycetes. While the present invention is not bound by a particular biological mechanism, it is believed that NLPs trigger plant defense responses in plants following recognition by a PRR of PAMPs derived from NLPs. Recently, the Arabidopsis RLP, AtRLP23, was identified as the PRR that recognizes the NLPs (Albert et al., 2015. Nat. Plants 1:15140, doi:10.1038/nplants.2015.140). It was shown by Albert et al. that in presence of AtRLP23, Arabidopsis plants can perceive NLP20 from Phytophthora infestans, and NLP24 peptides derived from various NLPs of Hyaloperonospora arabidopsidis (HaNLP3), Botrytis cinerea (BcNEP2), and Bacillus subtilis (BsNPP).

The methods for making engineered AtRLP23 proteins involve producing chimeric proteins comprising an LRR domain derived from AtRLP23 and a kinase domain derived from a receptor-like kinase (RLK). Such methods comprise producing a polypeptide comprising an amino acid sequence having the following domains in operable linkage and in an N-terminal-to-C-terminal direction: an LRR domain derived from an RLP that is capable of recognizing in a plant a pathogen-associated molecular pattern derived from an NLP, an extra-juxtamembrane (eJM) domain, a transmembrane (TM) domain, and a kinase domain derived from an RLK. If desired, the polypeptide can further comprise an SP domain that is operably linked to, and the N-terminal side of the LRR domain. Preferably, such an SP domain is capable of targeting a polypeptide to the plasmalemma of a plant cell when the SP domain is operably linked to the polypeptide.

It is recognized that when a polypeptide comprising an SP domain is expressed in a plant, the SP domain is typically cleaved by a signal peptidase shortly after translation. Thus, the engineered AtRLP23 proteins of the present invention include, not only the engineered AtRLP23 proteins disclosed herein comprising an SP domain, but also engineered AtRLP23 proteins lacking an SP domain. It is recognized that such engineered AtRLP23 proteins that do not comprise an SP domain include, for example, engineered AtRLP23 proteins that were initially synthesized in a plant cell or other host cell and comprised an SP domain that was later cleaved from the engineered AtRLP23 protein and any other engineered AtRLP23 protein lacking all or at least a part of an SP domain.

The methods of the present invention can comprise use of a domain derived from a particular protein of interest (e.g. an LRR domain derived from AtRLP23). By “derived from” it is intended that the domain has the same amino acid sequence as the native domain in the protein of interest or is a modified domain which comprises a modified amino acid sequence that is not identical to the full-length amino acid sequence of the native domain and which has the same biological function (e.g. PAMP recognition, membrane localization, kinase activity) as the native domain from the protein of interest or does not otherwise alter the biological function of the engineered AtRLP23 proteins, relative to the biological function of AtRLP23 (i.e. recognition of a PAMP derived from an NLP). Such a modified domain or variant domain is an artificial or non-naturally occurring protein domain that comprises a modified amino acid sequence and can be produced, for example, by modifying the amino acid sequence of a native domain or even two of more native domains or even my combining portions of two or more native domains. Such “modifying the amino acid sequence” comprises the substitution, addition, and/or deletion of one or more amino acids of the amino acid sequence of the native domain. Preferably, the engineered AtRLP23 proteins of the present invention are capable of recognizing in a plant a PAMP derived from an NLP and comprise increased activity or increased responsiveness when compared to the activity or responsiveness of AtRLP23 in one or more of the assays disclosed elsewhere herein or otherwise known in the art.

The engineered AtRLP23 proteins comprise an LRR domain derived from AtRLP23. In some embodiments of the invention, the LRR domain is the native LRR domain of AtRLP23 comprising the amino acid sequence set forth in SEQ ID NO: 560. In other embodiments, the LRR domain will comprise a variant AtRLP23 LRR domain that has at least about 50%, 55%, 60%, 65% 70%, 75%, 80%, 85%, 90%, 91%, 92%, 93%, 94%, 95%, 96%, 97%, 98%, 99% or more sequence identity to the amino acid sequence of the AtRLP23 LRR domain set forth in SEQ ID NO: 560. Preferably, such variants or modified AtRLP23 LRR domains will retain the biological function of the AtRLP23 LRR domain when such variants or modified AtRLP23 LRR domains are contained in an engineered AtRLP23 protein of the present invention—that is, an engineered AtRLP23 protein comprising a variant AtRLP23 LRR domain will be capable of recognizing in a plant a PAMP derived from an NLP. It is recognized that the biological function of a variant AtRLP23 LRR domain can be determined, for example, by replacing the native LRR domain of AtRLP23 with the variant LRR domain and assaying the activity or function of the chimeric protein by methods disclosed elsewhere herein.

Different kinase domains derived from RLK proteins can be used in the methods and compositions of the present invention. Examples of some RLK kinase domains that can be used in the methods and compositions of the present invention comprise the amino acid sequences set forth in SEQ ID NOS: 77-240 and 561-563, and variants thereof. In certain embodiments of the invention, an engineered AtRLP23 protein of the present invention will comprise a variant RLK kinase domain that has at least about 50%, 55%, 60%, 65% 70%, 75%, 80%, 85%, 90%, 91%, 92%, 93%, 94%, 95%, 96%, 97%, 98%, 99% or more sequence identity to one or more of the kinase domain amino acid sequences set forth in SEQ ID NOS: 77-240 and 561-563. In certain other embodiments of the invention, the kinase domain is derived from OsXA21, AtSOBIR1, AtPEPR1, or AtEFR. The amino acid sequences of the kinase domains of OsXA21, AtSOBIR1, AtPEPR1, and AtEFR are set forth in SEQ ID NOS: 113, 158, 562, and 189, respectively.

In some embodiments of the invention, the engineered AtRLP23 proteins comprise the native AtRLP23 SP, eJM, and TM domains. In other embodiments, the engineered AtRLP23 proteins comprise a polypeptide in which one or more of the SP, eJM, and TM domains is not identical to the corresponding native domain. Preferably, such non-native domains will retain the biological function of the corresponding AtRLP23 domain when such a non-native domains are contained in an engineered AtRLP23 protein of the present invention—that is, an engineered AtRLP23 protein comprising one or more of such non-native domains will be capable of recognizing in a plant a PAMP derived from an NLP. It is recognized that the biological function of a non-native SP, eJM, or TM domain can be determined, for example, by replacing the corresponding native domain of AtRLP23 with a non-native SP, eJM, or TM and assaying the activity or function of the chimeric protein by methods disclosed elsewhere herein.

The amino acid sequence of the native AtRLP23 eJM domain is set forth in SEQ ID NO: 543. Examples of some non-native eJM domains that can be used in the methods and compositions of the present invention comprise the amino acid sequences set forth in SEQ ID NOS: 241-390, 541, 542, 545-547, and 564-566, and variants thereof, and also variants of the amino acid sequence set forth in SEQ ID NO: 543. In certain embodiments of the invention, an engineered AtRLP23 protein of the present invention will comprise a variant eJM domain that has at least about 50%, 55%, 60%, 65% 70%, 75%, 80%, 85%, 90%, 91%, 92%, 93%, 94%, 95%, 96%, 97%, 98%, 99% or more sequence identity to one or more of the eJM domain amino acid sequences set forth in SEQ ID NOS: 241-390, 541-547, and 564-566. In certain other embodiments of the invention, the eJM is an eJM domain derived from an RLP, particularly a SOBIR1-dependent RLP including, but are not limited to, an eJM domain derived from AtRLP1 (SEQ ID NO: 542), AtRLP23 (SEQ ID NO: 543), AtRLP30 (SEQ ID NO: 544), AtRLP42 (SEQ ID NO: 545), Cf-4 (SEQ ID NO: 546), and/or Ve1 (SEQ ID NO: 547). While the naturally occurring versions of the eJM domain can be used in the methods and compositions, non-naturally occurring or synthetic eJM domains can be used such as, for example, eJM(EEEE/ADQ−) (SEQ ID NO: 541), which is a chimera of between the eJMs of AtRLP23 and AtRLP42 as described in Example 2 below.

As indicated above, the engineered AtRLP23 proteins produced by the methods of the present invention can comprise an SP domain and an TM domain. Preferably, the SP domain or is derived from a plasmalemma-bound protein. More preferably, the SP domain and the TM domain are each derived from a plasmalemma-bound protein. More preferably, the SP domain and the TM domain are derived from the same PRR or two different PRRs. In some preferred embodiments of the method of the invention, the SP domain is derived from AtRLP23 and the TM domain and the kinase domain are derived from AtEFR, AtPEPR1, or OsXA21. In other preferred embodiments, the SP, LRR, and TM domains are derived from AtRLP23 and the kinase domain is derived from AtSOBIR1.

The amino acid sequence of the native AtRLP23 TM domain is set forth in SEQ ID NO: 549. Examples of some non-native TM domains that can be used in the methods and compositions of the present invention comprise the amino acid sequences set forth in SEQ ID NOS: 391-540, 548, 550-553, and 567-569, and variants thereof, and also variants of the amino acid sequence set forth in SEQ ID NO: 549. In certain embodiments of the invention, an engineered AtRLP23 protein of the present invention will comprise a variant TM domain that has at least about 50%, 55%, 60%, 65% 70%, 75%, 80%, 85%, 90%, 91%, 92%, 93%, 94%, 95%, 96%, 97%, 98%, 99% or more sequence identity to one or more of the TM domain amino acid sequences set forth in SEQ ID NOS: 391-541, 548-553, and 567-569.

The amino acid sequence of the native AtRLP23 TM domain is set forth in SEQ ID NO: 555. Examples of some non-native SP domains that can be used in the methods and compositions of the present invention comprise the amino acid sequences set forth in SEQ ID NOS: 554, and 556-559 and variants thereof, and also variants of the amino acid sequence set forth in SEQ ID NO: 555. In certain embodiments of the invention, an engineered AtRLP23 protein of the present invention will comprise a variant SP domain that has at least about 50%, 55%, 60%, 65% 70%, 75%, 80%, 85%, 90%, 91%, 92%, 93%, 94%, 95%, 96%, 97%, 98%, 99% or more sequence identity to one or more of the TM domain amino acid sequences set forth in SEQ ID NOS: 554-559. Preferably, the SP domains that used in the engineered AtRLP23 proteins of the present invention are capable of targeting an operably linked engineered AtRLP23 protein to the plasmalemma of a plant cell.

Amino acid sequences encoding engineered AtRLP23 proteins produced by the methods of the present invention include, but are not limited to, the amino acid sequences set forth in SEQ ID NOS: 16, 20, 32, 40, and 60. In some embodiments of the invention, the engineered AtRLP23 proteins further comprise a 3×FLAG tag operably linked to the C-terminal end of the kinase domain. The 3×FLAG tags were added to certain of the engineered AtRLP23 proteins of the present invention to aid in detection and/or purification the engineered AtRLP23 proteins and are not believed to alter the biological function and/or membrane localization of the engineered AtRLP23 proteins. Examples of engineered AtRLP23 proteins of the present invention comprising a kinase domain and a 3×FLAG tag have the amino acid sequences set forth in SEQ ID NO: 18, 22, 34, 42, and 62.

The methods for making an engineered AtRLP23 protein comprise, or further comprise, modifying the amino acid sequence of the AtRLP23 protein whereby an engineered AtRLP23 protein is produced for which the portion of the amino acid sequence corresponding to the AtRLP23 protein (i.e. the amino acid sequence without the kinase domain and any adaptor or linker attached thereto) comprises an amino acid sequence that is not identical to the full-length amino acid sequence of AtRLP23. Such “modifying the amino acid sequence” comprises the substitution, addition, and/or deletion of one or more amino acids of amino acid sequence of the AtRLP23 protein. As noted above, a protein comprising a modified amino acid sequence is typically produced by modifying the nucleotide sequence of a nucleic acid molecule whereby a modified nucleotide sequence that encodes the desired modified amino acid sequence is produced and then by expressing the nucleotide sequence in a cell or in vitro, whereby a protein comprising the desired modified amino acid sequence is produced.

In some embodiments of the invention, the methods comprise replacing one or more of the SP, eJM, and TM domains of the AtRLP23 protein or part or parts thereof with the corresponding domain derived from another PRR or part or parts of such a corresponding domain.

In one embodiment of the invention, the engineered AtRLP23 protein comprises a modified eJM domain that is derived from the eJM domain of another RLP including, but not limited to, the native eJM domains of AtRLP1, AtRLP23, AtRLP30, AtRLP42, Cf-4, and Ve1 and modified versions thereof that do reduce the biological activity of the engineered AtRLP23 protein, relative to the activity of the AtRLP23 protein. In another embodiment of the invention, the engineered AtRLP23 protein comprises a modified eJM domain that is derived from the eJM domains of two or more PRRs. An example of such an engineered AtRLP23 protein is the AtRLP23-eJM(EEEE/ADQ−) protein (SEQ ID NO: 44) which comprises the modified eJM domain referred to herein as eJM(EEEE/ADQ−) (SEQ ID NO: 541) and which is described further in Example 2 below.

It is recognized that in making the engineered AtRLP23 proteins, it may be desirable or necessary to add adapters or linkers between any one or more of the various domains of such proteins to maintain or improve the biological activity or function of the engineered protein, relative to such an engineered protein lacking such an adaptor or linker. Such adapters or linkers may be employed to prohibit unwanted interactions between the discrete domains in a protein. Such adaptors or linkers can be oligopeptides (i.e. 2-20 amino acids), but in some situations, the adaptors and linkers can be relatively short polypeptides are between about 21 and 50 amino acids in length, preferably between 21-31 amino acids.

The methods of the present invention comprise producing an engineered AtRLP23 protein comprising a modified amino acid sequence. The engineered AtRLP23 protein can be produced, for example, by chemically synthesizing a polypeptide comprising the modified amino acid sequence or by producing a nucleic acid molecule encoding a polypeptide comprising the modified amino acid sequence and expressing the nucleotide acid molecule in a cell or in vitro, whereby an engineered AtRLP23 protein comprising the modified amino acid sequence is produced. It is recognized that such nucleic acid molecules can be produced, for example, by routine molecular biology methods disclosed elsewhere or otherwise known in the art or by chemical synthesis using a DNA synthesizer. Such molecular biology methods include, but are not limited to, gene editing, PCR amplification, cloning, site-directed mutagenesis, restriction nuclease enzyme digestion, ligation, and the like. It is further recognized that a nucleic acid molecule encoding an engineered AtRLP23 protein comprising a modified amino acid sequence can be produced within the genome of a plant cell or other host cell using genome-editing methods that are disclosed herein below or are otherwise known in the art.

Amino acid sequences encoding engineered AtRLP23 proteins produced by the methods of the present invention include, but are not limited to, the amino acid sequences set forth in SEQ ID NOS: 44, 48, 52, and 56. In some embodiments of the invention, the engineered AtRLP23 proteins further comprise a 3×FLAG tag operably linked to the C-terminal end of the engineered AtRLP23 protein. The 3×FLAG tags were added to certain of the engineered AtRLP23 proteins of the present invention to aid in detection and/or purification the engineered AtRLP23 proteins and are not believed to alter the biological function and/or membrane localization of the engineered AtRLP23 proteins. Examples of engineered AtRLP23 proteins of the present invention lacking a kinase but comprising a 3×FLAG tag have the amino acid sequences set forth in SEQ ID NO: 46, 50, 54, and 58.

The present invention further provides methods for making nucleic acid molecules encoding engineered AtRLP23 proteins of the present invention that are capable of conferring to a plant enhanced resistance to at least one plant pathogen. The methods comprise synthesizing a nucleic acid molecule encoding the amino acid sequence of an engineered AtRLP23 of the present invention. It is recognized that it is routine to make a nucleic acid molecule encoding a protein of interest and that such a nucleic acid molecule can be synthesized using, for example, a DNA synthesizer and/or using standard molecular biology methods described hereinbelow or otherwise known the art such as, for example, restriction endonuclease digestion, ligation, polymerase chain reaction (PCR) amplification, site-directed mutagenesis, sequencing, and the like. Examples of nucleic acid molecules encoding such engineered AtRLP23 proteins that are producible by the methods of the present invention are nucleic molecules comprising at least one of the nucleotide sequences set forth in SEQ ID NOS: 15, 17, 19, 21, 31, 33, 39, 41, 43, 45, 47, 49, 51, 53, 55, 57, 59, and 61.

The present invention provides not only engineered AtRLP23 proteins comprising a kinase domain, but also engineered AtRLP23 proteins lacking a kinase domain. Such engineered AtRLP23 proteins of the present invention that lack a kinase domain comprise or consist of an amino acid sequence that is not identical to the full-length amino acid sequence of AtRLP23 and can be produced by the methods disclosed herein.

The present invention further relates to compositions comprising at least one engineered AtRLP23 protein of the present invention and/or at least one nucleic acid molecule encoding such an engineered AtRLP23 protein. Such compositions include, but not limited to, plants, plant cells, and other host cells comprising one or more of such engineered AtRLP23 proteins and/or one or more nucleic acid molecules encoding such engineered AtRLP23 proteins, and expression cassettes and vectors comprising one or more of such nucleic acid molecules.

The present invention additionally provides methods for enhancing the resistance of a plant to a plant pathogen, particularly a plant comprising partial resistance to the plant pathogen. As used herein, full or complete resistance is defined as the inability of the pathogen to spread within the host plant genotype. With full resistance, localized cell death is observed on the plant after being contacted by the pathogen but there are no spreading lesions. In contrast with partial resistance, the pathogen may still be able to infect the host plant and cause a spreading lesion, but the spread of the lesion is restricted or limited, when compared to a susceptible plant.

Such methods for enhancing the resistance of a plant comprise modifying a plant cell to be capable of expressing (also referred to herein as overexpression) of at least one engineered AtRLP23 protein. The methods optionally further comprise regenerating the modified plant cell into a modified plant comprising enhanced resistance to the plant pathogen.

In some embodiments, the methods comprise introducing into at least one plant cell a polynucleotide construct comprising a promoter that drives expression in a plant and an operably linked nucleic acid molecule encoding the engineered AtRLP23 protein using plant transformation methods described elsewhere herein or otherwise known in the art. Preferred promoters for enhancing the resistance of a plant to a plant pathogen are promoters known to drive high-level gene expression such as, for example, the CaMV 35S promoter. Additional promoters that are suitable for use in the methods of the present invention are described hereinbelow.

The methods of the present invention find use in producing plants with enhanced resistance to a plant disease caused by a plant pathogen. Typically, the methods of the present invention will enhance or increase the resistance of the subject plant to one strains of a plant pathogen or to each of two or more strains of the plant pathogen by at least 25%, 50%, 75%, 100%, 150%, 200%, 250%, 500% or more when compared to the resistance of a control to same strain or strains of the plant pathogen. Unless stated otherwise or apparent from the context of a use, a control plant for the present invention is a plant that does not comprise the polynucleotide construct of the present invention. Preferably, the control plant is essentially identical (e.g. same species, subspecies, and variety) to the plant comprising the polynucleotide construction of the present invention accept the control does not comprise the polynucleotide construct. In some embodiments, the control plant will comprise a polynucleotide construct but not comprise one or more nucleotide sequences encoding an engineered AtRLP23 protein that are in a polynucleotide construct of the present invention. In other embodiments, the control plant will not comprise an engineered AtRLP23 protein of the present invention.

The plants of the present invention comprising an engineered AtRLP23 protein disclosed herein find use in methods for limiting plant disease caused by at least one plant pathogen in agricultural crop production, particularly in regions where such a plant disease is prevalent and is known to negatively impact, or at least has the potential to negatively impact, agricultural yield. The methods of the invention comprise planting a plant (e.g. a seedling), seed, or tuber of the present invention, wherein the plant, seed, or tuber comprises at least one engineered AtRLP23 protein of the present invention and/or at least one nucleotide molecule encoding engineered AtRLP23 protein. The methods further comprise growing the plant that is derived from the seedling, seed, or tuber under environmental conditions favorable for the growth and development of the plant, and optionally harvesting at least one fruit, tuber, leaf, or seed from the plant. Such environmental conditions can include, for example, air temperature, soil temperature, soil water content, photoperiod, light intensity, soil pH, and soil fertility. It is recognized that the environmental conditions favorable for the growth and development of a plant of interest will vary depending on, for example, the plant species or even the particular variety (e.g. cultivar) or genotype of the plant of interest. It is further recognized that the environmental conditions that are favorable for the growth and development of the plants of interest of the present invention are known in the art.

Additionally, the present invention provides plants, seeds, and plant cells produced by the methods of present invention and/or comprising a polynucleotide construct of the present invention. Also provided are progeny plants and seeds thereof comprising a polynucleotide construct of the present invention. The present invention also provides seeds, vegetative parts, and other plant parts produced by the transformed plants and/or progeny plants of the invention as well as food products and other agricultural products produced from such plant parts that are intended to be consumed or used by humans and other animals including, but not limited to pets (e.g., dogs and cats) and livestock (e.g., pigs, cows, chickens, turkeys, and ducks).

The present invention encompasses isolated or substantially purified polynucleotide (also referred to herein as “nucleic acid molecule”, “nucleic acid” and the like) or protein (also referred to herein as “polypeptide”) compositions including, for example, polynucleotides and proteins comprising the sequences set forth in the accompanying Sequence Listing as well as variants and fragments of such polynucleotides and proteins. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence protein encoded thereby. Fragments of polynucleotides comprising coding sequences may encode protein fragments that retain biological activity of the full-length or native protein. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the engineered AtRLP23 proteins of the invention. Variant polynucleotides include synthetically derived polynucleotides, such as those generated, for example, by using site-directed mutagenesis but which still encode an engineered AtRLP23 protein of the invention. Generally, variants of a polynucleotide of the invention will have at least about 80%, 85%, 90%, 91%, 92%, 93%, 94%, 95%, 96%, 97%, 98%, 99% or more sequence identity to that polynucleotide as determined by sequence alignment programs and parameters as described elsewhere herein. In certain embodiments of the invention, variants of a particular polynucleotide of the invention will have at least about 80%, 85%, 90%, 91%, 92%, 93%, 94%, 95%, 96%, 97%, 98%, 99% or more sequence identity to at least one nucleotide sequence selected from the group consisting of SEQ ID NOS: 15, 17, 19, 21, 31, 33, 39, 41, 43, 45, 47, 49, 51, 53, 55, 57, 59, and 61.

Variants of a polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptide of SEQ ID NO: 16, 18, 20, 22, 32, 34, 40, 42, 44, 46, 48, 50, 52, 54, 56, 58, 60, or 62 is disclosed. Percent sequence identity between any two polypeptides or between the corresponding parts (e.g. domains) of any two peptides can be calculated using sequence alignment programs and parameters described elsewhere herein. Where any given pair of polynucleotides of the invention or corresponding parts thereof is evaluated by comparison of the percent sequence identity shared by the two polypeptides they encode, the percent sequence identity between the two encoded polypeptides is at least about 50%, 55%, 60%, 65%, 70%, 75%, 80%, 85%, 86%, 87%, 88%, 89%, 90%, 91%, 92%, 93%, 94%, 95%, 96%, 97%, 98%, 99% or more sequence identity. In certain embodiments of the invention, variants of a particular polypeptide of the invention or domain there will have at least about 80%, 85%, 90%, 91%, 92%, 93%, 94%, 95%, 96%, 97%, 98%, 99% or more sequence identity to at least one of the full-length amino acid sequences set forth in SEQ ID NOS: 16, 18, 20, 22, 32, 34, 40, 42, 44, 46, 48, 50, 52, 54, 56, 58, 60, and 62. Preferably, the variants of a particular polypeptide of the invention will have one or more domains (e.g. SP domain, LRR domain, eJM domain, TM domain, kinase domain) that will have at least about 50%, 55%, 60%, 65% 70%, 75%, 80%, 85%, 90%, 91%, 92%, 93%, 94%, 95%, 96%, 97%, 98%, 99% or more sequence identity to the amino acid of the corresponding domain in the amino acid sequences set forth in SEQ ID NOS: 77-560.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. A biologically active variant of a protein of the invention may differ from that protein by as few as 1-15 amino acid residues, as few as 1-10, such as 6-10, as few as 5, as few as 4, 3, 2, or even 1 amino acid residue. Biologically active variants of an engineered AtRLP23 protein of the present invention will have at least about 80%, 85%, 86%, 87%, 88%, 89%, 90%, 91%, 92%, 93%, 94%, 95%, 96%, 97%, 98%, 99% or more sequence identity to the amino acid sequence for an engineered AtRLP23 protein set forth in the sequence listing (e.g. the amino acid sequence set forth in SEQ ID NO: 16, 18, 20, 22, 32, 34, 40, 42, 44, 46, 48, 50, 52, 54, 56, 58, 60, or 62) as determined by sequence alignment programs and parameters described elsewhere herein. Such biologically active variants of an engineered AtRLP23 protein of the present invention will typically comprise domains (e.g. SP domain, LRR domain, eJM domain, TM domain, kinase domain). The amino acid sequence of any one or more of the domains of such biologically active variants will comprise at least about 50%, 55%, 60%, 65% 70%, 75%, 80%, 85%, 90%, 91%, 92%, 93%, 94%, 95%, 96%, 97%, 98%, 99% or more sequence identity to the amino acid sequence of the corresponding domain of an engineered AtRLP23 protein set forth in the sequence listing as determined by sequence alignment programs and parameters described elsewhere herein. A biologically active variant of a protein of the invention or of a domain thereof may differ from that protein or domain by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by, for example, assays for monitoring transient changes in cytosolic Ca²⁺ concentration after the addition of an elicitor to a population of plant protoplasts expressing an engineered AtRLP23 protein. Such assays are described in detail in Examples 1-5 hereinbelow. Preferably, the engineered AtRLP23 proteins of the present invention comprise increased activity or increased responsiveness when compared to the activity or responsiveness of AtRLP23 (or AtRLP23-3×FLAG or other suitable control) in one or more of the assays disclosed elsewhere herein or otherwise known in the art.

For example, a plant that is susceptible to a plant disease caused by a plant pathogen of interest can be transformed with a polynucleotide construct encoding an engineered AtRLP23 protein of the present invention, regenerated into a transformed or transgenic plant comprising the polynucleotide constructs, and tested for resistance using standard disease resistance assays known in the art or described elsewhere herein.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997)J Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Preferably, the engineered AtRLP23 proteins of the present invention and the polynucleotides encoding them confer, or are capable of conferring, upon a plant comprising such a protein or polynucleotide, enhanced resistance to at least one plant pathogen, but preferably to two, three, four, five, or more plant pathogens.

PCR amplification can be used in certain embodiments of the methods of the present invention. Methods for designing PCR primers and PCR amplification are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR amplification include, but are not limited to, methods using paired primers, nested primers, single specific primers, degenerate primers, gene-specific primers, vector-specific primers, partially mismatched primers, and the like.

It is recognized that the nucleic acid molecules encoding an engineered AtRLP23 protein of the present invention encompass nucleic acid molecules comprising a nucleotide sequence that is sufficiently identical to the nucleotide sequence of SEQ ID NO: 15, 17, 19, 21, 31, 33, 39, 41, 43, 45, 47, 49, 51, 53, 55, 57, 59, and/or 61. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain(s) and/or common functional activity, such as, for example, disease resistance. For example, amino acid or nucleotide sequences that contain a common structural domain(s) and/or sequences having at least about 45%, 55%, or 65% identity, preferably 75% identity, more preferably 85%, 86%, 87%, 88%, 89%, 90%, 95%, 96%, 97%, 98% or 99% identity, can be as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and JessAltschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and)(BLAST programs of Altschul et al. (1990) J Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the)(BLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST; available on the world-wide web at ncbi.nlm.nih.gov). Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, Md., USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website on the world-wide web at: ebi.ac.uk/Tools/clustalw/index.html).

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotide constructs comprising engineered AtRLP23 protein coding regions can be provided in expression cassettes for expression in the plant or other organism or in a host cell of interest. The cassette will include 5′ and 3′ regulatory sequences operably linked to the protein coding region. “Operably linked” is intended to mean a functional linkage between two or more elements. For example, an operable linkage between a polynucleotide or gene of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the protein coding region to be under the transcriptional regulation of the regulatory regions. The expression cassette may additionally contain selectable marker genes.

The expression cassette will include in the 5′-3′ direction of transcription, a transcriptional and translational initiation region (i.e., a promoter), an engineered AtRLP23 protein coding region of the invention, and a transcriptional and translational termination region (i.e., termination region) functional in plants or other organism or non-human host cell. The regulatory regions (i.e., promoters, transcriptional regulatory regions, and translational termination regions) and/or the engineered AtRLP23 protein coding region or of the invention may be native/analogous to the host cell or to each other. Alternatively, the regulatory regions and/or the engineered AtRLP23 protein coding region of the invention may be heterologous to the host cell or to each other.

As used herein, “heterologous” in reference to a nucleic acid molecule or nucleotide sequence is a nucleic acid molecule or nucleotide sequence that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The present invention provides host cells comprising at least of the nucleic acid molecules, expression cassettes, and vectors of the present invention. In preferred embodiments of the invention, a host cell is a plant cell. In other embodiments, a host cell is selected from the group consisting of a bacterium, a fungal cell, and an animal cell. In certain embodiments, a host cell is non-human animal cell. However, in some other embodiments, the host cell is an in-vitro cultured human cell.

The termination region may be native with the transcriptional initiation region, may be native with the operably linked engineered AtRLP23 protein coding region of interest, may be native with the plant host, or may be derived from another source (i.e., foreign or heterologous to the promoter, the protein of interest, and/or the plant host), or any combination thereof. Convenient termination regions are available from the Ti-plasmid of A. tumefaciens, such as the octopine synthase and nopaline synthase termination regions. See also Guerineau et al. (1991)Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

Additionally, the polynucleotides can be modified to alter the amino acid sequences of the engineered AtRLP23 proteins, for example, to improve translational efficiency, protein stability and/or any other desired property or properties, and/or to reduce any one or more undesirable properties, while improving or at least not reducing significantly the biological activity of the engineered AtRLP23 proteins. For example, the polynucleotides can be modified to remove potential allergenic regions in the proteins encoded thereby. See, the AllergenOnline database for a comprehensive list of known and putative allergens (Goodman et al. (2016) Mol. Nutr. Food Res. 60(5):1183-1198; available on the World Wide Web at: allergenonline.org).

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters (also referred to as “adaptors) or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mot. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the engineered AtRLP23 protein coding sequences within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant 4(3):495-505. Such promoters can be modified, if necessary, for weak expression.

The transgene can be expressed using an inducible promoter, such as, for example, a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990)Mol. Cell. Biol. 10:3343-3356; Zambretti et al. (1992) Proc. Natl. Acad. Sci. USA 89:3952-3956; Bairn et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not intended to be limiting. Any selectable marker gene can be used in the present invention.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P:119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

Plant transformation vectors that find use in the present invention include, for example, T-DNA vectors or plasmids, which are suitable for use in Agrobacterium-mediated transformation methods that are disclosed elsewhere herein or otherwise known in the art.

The methods of the invention involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In specific embodiments, the polynucleotide constructs and expression cassettes of the invention can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91: 2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described elsewhere herein.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

Such methods known in the art for modifying DNA in the genome of a plant include, for example, mutation breeding and genome editing techniques, such as, for example, methods involving targeted mutagenesis, site-directed integration (SDI), and homologous recombination. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for gene modification or gene replacement comprising homologous recombination can involve inducing single-strand or double-strand breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Res. 33:5978-90; Mani et al. (2005) Biochem. Biophys. Res. Comm 335:447-57; U.S. Pat. Nos. 7,163,824, 7,001,768, and 6,453,242; Arnould et al. (2006) J Mol. Biol. 355:443-58; Ashworth et al., (2006) Nature 441:656-9; Doyon et al. (2006) J Am Chem Soc 128:2477-84; Rosen et al., (2006) Nucleic Acids Res. 34:4791-800; and Smith et al., (2006) Nucleic Acids Res. 34:e149; U.S. Pat. App. Pub. No. 2009/0133152; and U.S. Pat. App. Pub. No. 2007/0117128; all of which are herein incorporated in their entirety by reference.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze and Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

The CRISPR/Cas nuclease system can also be used to make single-strand or double-strand breaks at specific recognition sequences in the genome of a plant for gene modification or gene replacement through homologous recombination. The CRISPR/Cas nuclease is an RNA-guided (simple guide RNA, sgRNA in short) DNA endonuclease system performing sequence-specific double-stranded breaks in a DNA segment homologous to the designed RNA. It is possible to design the specificity of the sequence (Cho S. W. et al., Nat. Biotechnol. 31:230-232, 2013; Cong L. et al., Science 339:819-823, 2013; Mali P. et al., Science 339:823-826, 2013; Feng Z. et al., Cell Research: 1-4, 2013).

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov F. D. et al., Nat Rev Genet. 11:636-46, 2010; Carroll D., Genetics. 188:773-82, 2011).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

Unless expressly stated or apparent from the context of usage, the methods and compositions of the present invention can be used with any plant species including, for example, monocotyledonous plants, dicotyledonous plants, and conifers. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), triticale (x Triticosecale or Triticum x Secale) sorghum (Sorghum bicolor, Sorghum vulgare), teff (Eragrostis tej), millet (e.g., pearl millet (Pennisetum glaucum), proso millet (Panicum miliaceum), foxtail millet (Setaria italica), finger millet (Eleusine coracana)), switchgrass (Panicum virgatum), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), strawberry (e.g. Fragaria x ananassa, Fragaria vesca, Fragaria moschata, Fragaria virginiana, Fragaria chiloensis), sweet potato (Ipomoea batatus), yam (Dioscorea spp., D. rotundata, D. cayenensis, D. alata, D. polystachya, D. bulbifera, D. esculenta, D. dumetorum, D. trifida), cassava (Manihot esculenta), coffee (Coffea spp.), coconut (Cocos nucifera), oil palm (e.g. Elaeis guineensis, Elaeis oleifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), date (Phoenix dactylifera), cultivated forms of Beta vulgaris (sugar beets, garden beets, chard or spinach beet, mangelwurzel or fodder beet), sugarcane (Saccharum spp.), oat (Avena sativa), barley (Hordeum vulgare), cannabis (Cannabis sativa, C. indica, C. ruderalis), poplar (Populus spp.), eucalyptus (Eucalyptus spp.), Arabidopsis thaliana, Arabidopsis rhizogenes, Nicotiana benthamiana, Brachypodium distachyon vegetables, ornamentals, and conifers and other trees. In specific embodiments, plants of the present invention are crop plants (e.g. maize, sorghum, wheat, millet, rice, barley, oats, sugarcane, alfalfa, soybean, peanut, sunflower, cotton, safflower, Brassica spp., lettuce, strawberry, apple, citrus, etc.).

Vegetables include tomatoes (Lycopersicon esculentum), eggplant (also known as “aubergine” or “brinjal”) (Solanum melongena), pepper (Capsicum annuum), lettuce (e.g., Lactuca sativa), green beans (Phaseolus vulgaris), lima beans (Phaseolus limensis), peas (Lathyrus spp.), chickpeas (Cicer arietinum), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Fruit trees and related plants include, for example, apples, pears, peaches, plums, oranges, grapefruits, limes, pomelos, palms, and bananas. Nut trees and related plants include, for example, almonds, cashews, walnuts, pistachios, macadamia nuts, filberts, hazelnuts, and pecans.

In specific embodiments, the plants of the present invention are crop plants such as, for example, maize (corn), soybean, wheat, rice, cotton, alfalfa, sunflower, canola (Brassica spp., particularly Brassica napus, Brassica rapa, Brassica juncea), rapeseed (Brassica napus), sorghum, millet, barley, triticale, safflower, peanut, sugarcane, tobacco, potato, tomato, and pepper.

The term “plant” is intended to encompass plants at any stage of maturity or development, as well as any cells, tissues or organs (plant parts) taken or derived from any such plant unless otherwise clearly indicated by context. Plant parts include, but are not limited to, fruits, stems, tubers, roots, flowers, ovules, stamens, petals, leaves, hypocotyls, epicotyls, cotyledons, embryos, meristematic regions, callus tissue, anther cultures, gametophytes, sporophytes, pollen, microspores, protoplasts, seeds, and the like. It is recognized that the plant protoplasts of the present invention can be prepared from any one or more of the aforementioned plant parts and at any stage of development and/or maturity.

Likewise, the term “plant cell” is intended to encompass plant cells obtained from or in plants at any stage of maturity or development unless otherwise clearly indicated by context. Plant cells can be from or in plant parts including, but are not limited to, fruits, stems, tubers, roots, flowers, ovules, stamens, leaves, embryos, meristematic regions, callus tissue, anther cultures, gametophytes, sporophytes, pollen, microspores, in vitro-cultured tissues, organs or cells and the like. It is recognized that the plant protoplasts of the present invention can be prepared from any one or more of the aforementioned plant cells and at any stage of development and/or maturity. As used herein, unless expressly stated otherwise or apparent from the context of usage, the term “plant cell” is intended to encompass a plant protoplast.

In some embodiments of the present invention, a plant cell is transformed with a polynucleotide construct encoding an engineered an engineered AtRLP23 protein of the present invention. The term “expression” as used herein refers to the biosynthesis of a gene product, including the transcription and/or translation of said gene product. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide. Examples of polynucleotide constructs and nucleic acid molecules that encode engineered AtRLP23 proteins are described elsewhere herein.

The use of the terms “DNA” or “RNA” herein is not intended to limit the present invention to polynucleotide molecules comprising DNA or RNA. Those of ordinary skill in the art will recognize that the methods and compositions of the invention encompass polynucleotide molecules comprised of deoxyribonucleotides (i.e., DNA), ribonucleotides (i.e., RNA) or combinations of ribonucleotides and deoxyribonucleotides. Such deoxyribonucleotides and ribonucleotides include both naturally occurring molecules and synthetic analogues including, but not limited to, nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The polynucleotide molecules of the invention also encompass all forms of polynucleotide molecules including, but not limited to, single-stranded forms, double-stranded forms, hairpins, stem-and-loop structures, and the like. Furthermore, it is understood by those of ordinary skill in the art that the nucleotide sequences disclosed herein also encompasses the complement of that exemplified nucleotide sequence.

The invention is drawn to compositions and methods for producing a plant with enhanced resistance to a plant disease caused by one, two, three, four or more plant pathogens. By “resistance to a plant disease” or “disease resistance” is intended that the plants avoid the disease symptoms that are the outcome of plant-pathogen interactions. That is, one or more pathogens are prevented from causing a plant disease or plant diseases and the associated disease symptoms, or alternatively, the disease symptoms caused by the one or more pathogens is minimized or lessened.

The present invention encompasses the polynucleotide constructs disclosed herein or in the accompanying sequence listing and/or drawings including, but not limited to, a polynucleotide construct comprising a nucleotide sequence selected from the group consisting of SEQ ID NOS: 15, 17, 19, 21, 31, 33, 39, 41, 43, 45, 47, 49, 51, 53, 55, 57, 59, and 61 and variants thereof as disclosed elsewhere herein. The present invention further encompasses plants, plant cells, host cells, and vectors comprising at least one of such polynucleotide constructs, as well as food products produced from such plants. Additionally encompassed by the present invention are uses of plants comprising at least one of such polynucleotide constructs in the methods disclosed elsewhere herein such as, for example, methods of limiting plant diseases in agricultural crop production.

Plant pathogens include, for example, bacteria, fungi, oomycetes, viruses, nematodes, and the like. Specific pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassicicola, Pythium ultimum, Peronospora parasitica, Fusarium roseum, Alternaria alternata; Alfalfa: Clavibacter michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Verticillium albo-atrum, Xanthomonas campestris p.v. alfalfae, Aphanomyces euteiches, Stemphylium herbarum, Stemphylium alfalfae, Colletotrichum trifolii, Leptosphaerulina briosiana, Uromyces striatus, Sclerotinia trifoliorum, Stagonospora meliloti, Stemphylium botryosum, Leptotrichila medicaginis; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f. sp. tritici, Puccinia graminis f sp. tritici, Puccinia recondita f sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Plasmopora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Colletotrichum graminicola, Fusarium moniliforme var. subglutinans, Erwinia stewartii, Gibberella zeae (Fusarium graminearum), Fusarium verticilloides, Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, C. sublineolum, Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium verticillioides, Fusarium oxysporum, Pythium arrhenomanes, Pythium graminicola, etc.; Tomato: Corynebacterium michiganense pv. michiganense, Pseudomonas syringae pv. tomato, Ralstonia solanacearum, Xanthomonas vesicatoria, Xanthomonas perforans, Alternaria solani, Alternaria porri, Collectotrichum spp., Fulvia fulva Syn. Cladosporium fulvum, Fusarium oxysporum f. lycopersici, Leveillula taurica/Oidiopsis taurica, Phytophthora infestans, other Phytophthora spp., Pseudocercospora fuligena Syn. Cercospora fuligena, Sclerotium rolfsii, Septoria lycopersici, Meloidogyne spp.; Potato: Ralstonia solanacearum, Pseudomonas solanacearum, Erwinia carotovora subsp. Atroseptica Erwinia carotovora subsp. Carotovora, Pectobacterium carotovorum subsp. Atrosepticum, Pseudomonas fluorescens, Clavibacter michiganensis subsp. Sepedonicus, Corynebacterium sepedonicum, Streptomyces scabiei, Colletotrichum coccodes, Alternaria alternate, Mycovellosiella concors, Cercospora solani, Macrophomina phaseolina, Sclerotium bataticola, Choanephora cucurbitarum, Puccinia pittieriana, Aecidium cantensis, Alternaria solani, Fusarium spp., Phoma solanicola f. foveata, Botrytis cinerea, Botryotinia fuckeliana, Phytophthora infestans, Pythium spp., Phoma andigena var. andina, Pleospora herbarum, Stemphylium herbarum, Erysiphe cichoracearum, Spongospora subterranean Rhizoctonia solani, Thanatephorus cucumeris, Rosellinia sp. Dematophora sp., Septoria lycopersici, Helminthosporium solani, Polyscytalum pustulans, Sclerotium rolfsii, Athelia rolfsii, Angiosorus solani, Ulocladium atrum, Verticillium albo-atrum, V. dahlia, Synchytrium endobioticum, Sclerotinia sclerotiorum, Candidatus Liberibacter solanacearum; Banana: Fusarium oxysporum f. sp. cubense, Colletotrichum musae, Armillaria mellea, Armillaria tabescens, Pseudomonas solanacearum, Phyllachora musicola, Mycosphaerella fijiensis, Rosellinia bunodes, Pseudomas spp., Pestalotiopsis leprogena, Cercospora hayi, Pseudomonas solanacearum, Ceratocystis paradoxa, Verticillium theobromae, Trachysphaera fructigena, Cladosporium musae, Junghuhnia vincta, Cordana johnstonii, Cordana musae, Fusarium pallidoroseum, Colletotrichum musae, Verticillium theobromae, Fusarium spp., Acremonium spp., Cylindrocladium spp., Deightoniella torulosa, Nattrassia mangiferae, Dreschslera gigantean, Guignardia musae, Botryosphaeria ribis, Fusarium solani, Nectria haematococca, Fusarium oxysporum, Rhizoctonia spp., Colletotrichum musae, Uredo musae, Uromyces musae, Acrodontium simplex, Curvularia eragrostidis, Drechslera musae-sapientum, Leptosphaeria musarum, Pestalotiopsis disseminate, Ceratocystis paradoxa, Haplobasidion musae, Marasmiellus inoderma, Pseudomonas solanacearum, Radopholus similis, Lasiodiplodia theobromae, Fusarium pallidoroseum, Verticillium theobromae, Pestalotiopsis palmarum, Phaeoseptoria musae, Pyricularia grisea, Fusarium moniliforme, Gibberella fujikuroi, Erwinia carotovora, Erwinia chrysanthemi, Cylindrocarpon musae, Meloidogyne arenaria, Meloidogyne incognita, Meloidogyne javanica, Pratylenchus coffeae, Pratylenchus goodeyi, Pratylenchus brachyurus, Pratylenchus reniformia, Sclerotinia sclerotiorum, Nectria foliicola, Mycosphaerella musicola, Pseudocercospora musae, Limacinula tenuis, Mycosphaerella musae, Helicotylenchus multicinctus, Helicotylenchus dihystera, Nigrospora sphaerica, Trachysphaera frutigena, Ramichloridium musae, Verticillium theobromae, Phytophthora infestans, Phytophthora parasitica, Phytophthora ramorum, Phytophthora ipomoeae, Phytophthora mirabilis, Phytophthora capsici, Phytophthora porri, Phytophthora sojae, Phytophthora palmivora, and Phytophthora phaseoli.

Bacterial pathogens include, but are not limited to, i Agrobacterium tumefaciens, Candidatus Liberibacter asiaticus, Candidatus Liberibacter solanacearum, Clavibacter michiganensis, Clavibacter sepedonicus, Dickeya dadantii, Dickeya solani, Erwinia amylovora, Pectobacterium atrosepticum, Pectobacterium carotovorum, Pseudomonas andropogonis, Pseudomonas avenae, Pseudomonas alboprecipitans, Pseudomonas fluorescens, Pseudomonas savastanoi, Pseudomonas solanacearum, Pseudomonas syringae, Ralstonia solanacearum, Xanthomonas axonopodis, Xanthomonas campestris, Xanthomonas citri, Xanthomonas perforans, Xanthomonas vesicatoria, Xanthomonas oryzae, and Xylella fastidiosa.

Oomycete pathogens include, but are not limited to, Phytophthora infestans, Phytophthora ipomoeae, Phytophthora mirabilis, Phytophthora phaseoli, Phytophthora megasperma fsp. glycinea, Phytophthora megasperma, Phytophthora cryptogea, Peronospora spp. and Pythium spp.

Additional embodiments of the methods and compositions of the present invention are described elsewhere herein.

The following examples are offered by way of illustration and not by way of limitation.

### EXAMPLES

### Example 1: Heterologous Expression of AtRLP23-3×FLAG in Corn Confers Perception of PpNLP20

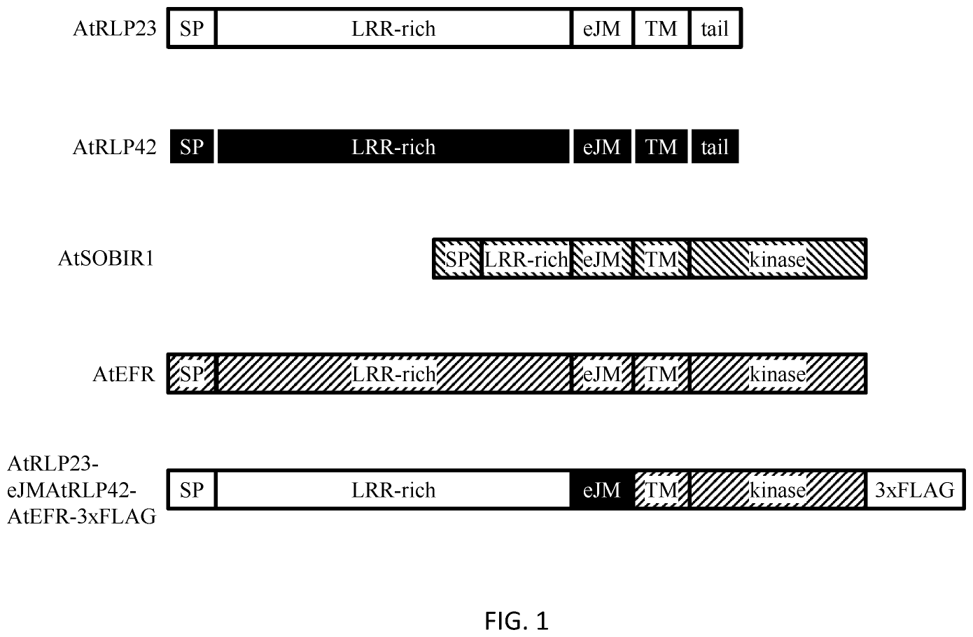

Plants employ receptor-like kinases (RLKs) and receptor-like proteins (RLPs) as pattern recognition receptors (PRRs) to monitor their apoplastic environment and detect non-self and damaged-self patterns as signs of potential danger (Boutrot and Zipfel, 2017. Annu. Rev. Phytopathol. 55:257, doi:10.1146/annurev-phyto-080614-120106). RLKs and RLPs contain a ligand-binding ectodomain and a single-pass transmembrane domain. RLKs have an intracellular kinase domain, whereas RLPs lack any obvious intracellular signalling domains (FIG. 1).

Following ligand (also known as elicitor) binding, PRRs are activated and induce a transient increase of cytosolic Ca²⁺ concentration as part of a PTI response. The transient increase of cytosolic Ca²⁺ concentration in response to a given elicitor can be monitored in a population of plant protoplasts expressing a calcium-binding fluorescent protein such as RGECO1.2 or luminescent protein such as aequorin. In a high-throughput method, the kinetics of PRR-dependent transient variation of cytosolic Ca²⁺ concentration can be measured with a fluorimeter or luminometer and in microplate formats of 96 or 384 wells.

The Necrosis- and Ethylene-inducing Protein 1 (Nep1)-like Protein (NLP) family of PAMPs are found in bacteria, fungi, and oomycetes (Bohm et al., 2014. PLoS Pathog. 10:e1004491, doi:10.1371/journal.ppat.1004491; Oome et al., 2014. Proc. Natl. Acad. Sci. USA 111:16955, doi:10.1073/pnas.1410031111; Albert et al., 2015. Nat. Plants 1:15140, doi:10.1038/nplants.2015.140). The 24-kDa Nep1 protein was originally discovered in the fungus Fusarium oxysporum (Bailey, 1995. Cell Biol. 85:1250, doi:10.1094/Phyto-85-1250) and NLPs were later identified by homology in the oomycetes Pythium aphanidermatum (Veit et al., 2001. Plant Physiol. 127:832, doi:10.1104/pp. 010350) and Phytophthora sojae (Qutob et al., 2002. Plant 32:361, doi:10.1046/j.1365-313X.2002.01439.x) as well as in the bacteria Bacillus halodurans and Streptomyces coelicolor (Qutob et al., 2002. Plant J. 32:361, doi:10.1046/j.1365-313X.2002.01439.x) and Erwinia carotovora (Pemberton et al., 2005. Mol. Plant Microbe. Interact. 18:343, doi:10.1094/MPMI-18-0343).

NLPs carry a conserved pattern of 24 amino acids (NLP24) which is sufficient to trigger plant immune responses (Böhm et al., 2014. PLoS Pathog. 10:e1004491, doi:10.1371/journal.ppat.1004491; Oome et al., 2014. Proc. Natl. Acad. Sci. USA 111:16955, doi:10.1073/pnas.1410031111; Albert et al., 2015. Nat. Plants 1:15140, doi:10.1038/nplants.2015.140). Within this 24-amino acid region, smaller epitopes have also the ability to induce defence responses in plants (Böhm et al., 2014. PLoS Pathog. 10:e1004491, doi:10.1371/journal.ppat.1004491; Oome et al., 2014. Proc. Natl. Acad. Sci. USA 111:16955, doi:10.1073/pnas.1410031111; Albert et al., 2015. Nat. Plants 1:15140, doi:10.1038/nplants.2015.140). A conserved 20-mer fragment (NLP20) derived from Phytophthora parasitica (PpNLP20; SEQ ID NO: 63) was confirmed to have a potent eliciting activity in Arabidopsis (Böhm et al., 2014. PLoS Pathog. 10:e1004491, doi:10.1371/journal.ppat.1004491).

Despite their diverse phylogenetic distribution, NLPs share a high degree of sequence similarity and several members of the family have the remarkable ability to induce cell death in as many as 20 dicotyledonous plants (Pemberton and Salmond, 2004. Mol. Plant Pathol. 5:353, doi:10.1111/j.1364-3703.2004.00235.x). Interestingly, no monocotyledonous plant tested produced any detectable response when treated with the protein NEP1 purified from F. oxysporum (tested in i Zea mays, Triticum aestivum, Stenotaphrum secundatum and Phalaris arundinacea) (Bailey, 1995. Cell Biol. 85:1250, doi:10.1094/Phyto-85-1250) or to the recombinant protein PaNie from Pythium aphanidermatum (tested in Zea mays, Avena sativa and Tradescantia zebrina) (Veit et al., 2001. Plant Physiol. 127:832, doi:10.1104/pp. 010350).

Recently, the Arabidopsis receptor-like protein (RLP) AtRLP23 was identified as the PRR that recognizes the NLPs (Albert et al., 2015. Nat. Plants 1:15140, doi:10.1038/nplants.2015.140). In presence of AtRLP23, Arabidopsis plants can perceive NLP20 from Phytophthora infestans, and NLP24 peptides derived from various NLPs of Hyaloperonospora arabidopsidis (HaNLP3), Botrytis cinerea (BcNEP2), and Bacillus subtilis (BsNPP).

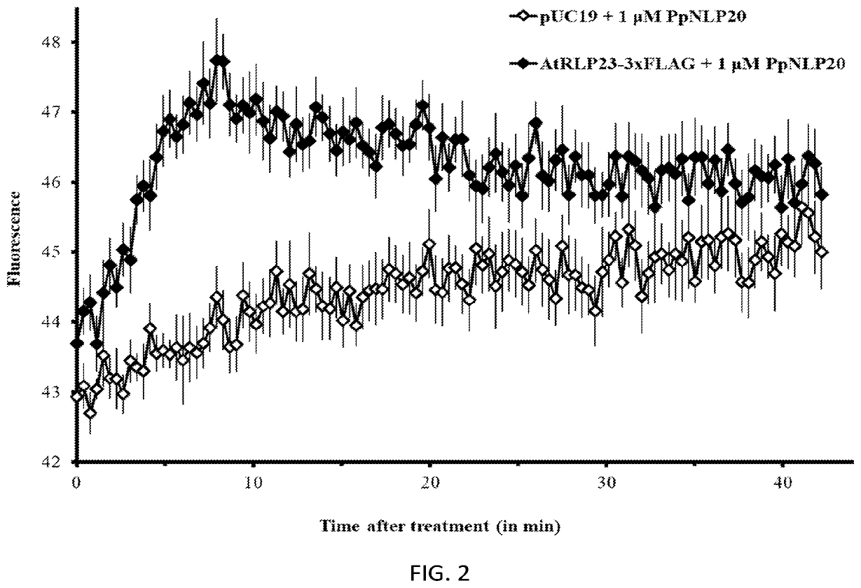

To examine the function of AtRLP23 in corn, corn protoplasts were isolated from leaves of 10-day old maize seedlings that had been kept in the dark at 25° C. essentially as described in Sheen et al. (1990) Plant Cell 2:1027-1038. 32×10⁴ corn (Zea mays) protoplasts were co-transfected with either 10 μg of the AtRLP23-3×FLAG construct (SEQ ID NO: 13) under the control of an operably linked 2×35S+Ω promoter construct (SEQ ID NO: 72) and containing an operably linked rbcS terminator (SEQ ID NO: 73) or 10 μg of control DNA (pUC19, SEQ ID NO: 74), and 10 μg of reporter construct (ZmUbi::R-GECO1.2::rbcS, SEQ ID NO: 75) by PEG-mediated transformation using an adaptation of the method of Yoo et al. (2007) Nat Protocols 2:1565-1572. 100 μL of transfected protoplasts (32×10⁴ protoplasts) were transferred to each well of a white 96-well microplate (Greiner Bio-One, Lumitrac, model 655075), and then 50 μL of 3 μM PpNLP20 peptide (AIMYSWYFPKDSPVTGLGHR, SEQ ID NO: 63; prepared by diluting a 100 μM stock solution with protoplast incubation buffer) was added to each well. Fluorescence was monitored using a fluorimeter equipped with a 100 Hz xenon flash lamp with excitation at 556 nm and emission measured at 585 nm for 100 ms, every 22.6 seconds for 42 minutes. As shown in FIG. 2, transient expression of AtRLP23-3×FLAG in corn protoplasts led to a significant Ca²⁺ increase in response to PpNLP20 treatment. These results demonstrate that AtRLP23-3×FLAG expression in corn protoplast leads to PpNLP20 perception.

### Example 2: Swapping the Extra-Juxtamembrane (eJM) Domain of AtRLP23 with Another eJM Domain Improves Activity

Recent evidence indicates that AtRLP23 constitutively interacts with the RLK, AtSOBIR1 (Gust and Felix, 2014. Curr. Opin. Plant Biol. 21:104, doi:10.1016/j.pbi.2014.07.007). As AtSOBIR1 only harbors a short ectodomain, an RLP/SOBIR1 heterodimer may function as a bi-modular RLK where the RLP ectodomain recognizes the elicitor and the SOBIR1 kinase domain functions in intracellular signalling (Gust and Felix, 2014. Curr. Opin. Plant Biol. 21:104, doi:10.1016/j.pbi.2014.07.007). Interestingly, all the RLPs from the Leucine-Rich Repeat family that are known to function in a SOBIR1-dependent manner (i.e. AtRLP1, AtRLP23, AtRLP30, AtRLP42, Cf-4, and Ve1 (Shibuya and Desaki, 2015. Nat. Plants 1:15149, doi:10.1038/nplants.2015.149)) contain a highly negatively charged eJM, while the SOBIR1 eJM is highly positively charged. These complementary charges may be important for the physical interaction and stabilisation of the RLPs/SOBIR1 heterodimers (Gust and Felix, 2014. Curr. Opin. Plant Biol. 21:104, doi:10.1016/j.pbi.2014.07.007).

We engineered a series of chimeric AtRLP23 variants which contain an eJM domain chosen from the following three SOBIR1-dependent RLPs: Ve1 (from Solanum lycopersicum, GenBank: AAK58682.1), AtRLP1 (AT1G07390.4, from Arabidopsis thaliana Col-0), AtRLP42 (AT3G25020.1 from Arabidopsis thaliana Col-0). A fourth eJM, present in the construct AtRLP23-eJM(EEEE/ADQ−)-3×FLAG (SEQ ID NO: 45), is a chimera between the eJMs of AtRLP23 and AtRLP42.

32×10⁴ corn protoplasts, that were prepared as described in Example 1, were co-transfected with 10 μg of reporter construct (ZmUbi::R-GECO1.2::rbcS, SEQ ID NO: 75) and 10 μg of one of the following AtRLP23 constructs: AtRLP23-3×FLAG (SEQ ID NO: 13), AtRLP23-eJM(EEEE/ADQ−)-3×FLAG (SEQ ID NO: 45), AtRLP23-eJMAtRLP1-3×FLAG (SEQ ID NO: 49), AtRLP23-eJMAtRLP42-3×FLAG (SEQ ID NO: 57) or AtRLP23-eJMVe1-3×FLAG (SEQ ID NO: 53). All of the AtRLP23 constructs have an operably linked 2×35S+Ω promoter construct (SEQ ID NO: 72) and an operably linked rbcS terminator (SEQ ID NO: 73)

100 μL of protoplasts (32×10⁴ protoplasts) were transferred to each well of a white 96-well microplate (Greiner Bio-One, Lumitrac, model 655075), and then 50 μL of protoplast incubation buffer or 3 μM PpNLP20 peptide (AIMYSWYFPKDSPVTGLGHR, SEQ ID NO: 63; prepared by diluting a 100 μM stock solution with protoplast incubation buffer) was added to each well. Fluorescence was monitored using a fluorimeter equipped with a 100 Hz xenon flash lamp with excitation at 556 nm and emission measured at 585 nm for 30 ms, every 20.2 seconds for 40 minutes. For each fluorescence measurement, the transient change in R-GECO1.2 fluorescence (ΔF) are calculated from background corrected intensity values as (F-Fo), where F represents the average fluorescence intensity of the a batch of protoplasts treated with elicitor, and Fo represents the average fluorescence intensity of the same batch of protoplasts treated with a control solution that lacks the elicitor but is otherwise identical or essentially identical in composition to the solution comprising the elicitor.

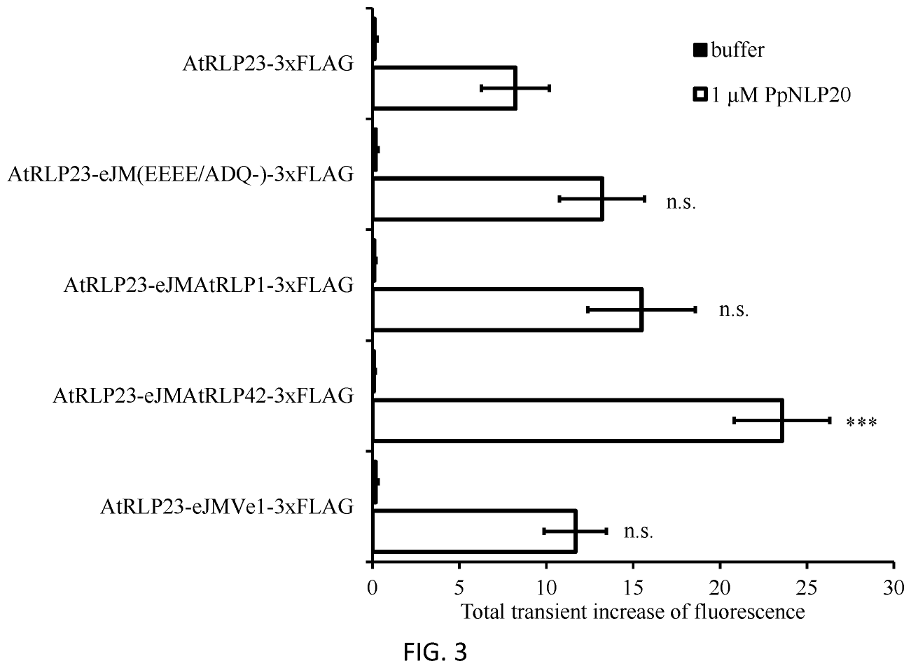

As shown in FIG. 3, transient expression of AtRLP23-3×FLAG, AtRLP23-eJM(EEEE/ADQ−)-3×FLAG, AtRLP23-eJMAtRLP1-3×FLAG, AtRLP23-eJMAtRLP42-3×FLAG or AtRLP23-eJMVe1-3×FLAG, in corn protoplasts led to a Ca²⁺ increase in response to PpNLP20 treatment. Following PpNLP20 treatment, the transient expression of AtRLP23-eJMAtRLP42-3×FLAG conferred a statistically significant increase of response compared to the transient expression of AtRLP23-3×FLAG.

### Example 3: Fusing the AtRLP23 Ectodomain to the Kinase Domain of an RLK can Improve the Activity of AtRLP23

The functionality of several Leucine-Rich Repeat (LRR)-type RLPs depends on their association with the common adaptor kinase SOBIR1. These RLP/adaptor complexes, formed in the absence of ligands, have been described as bimolecular equivalents of RLKs (Gust and Felix, 2014. Curr. Opin. Plant Biol. 21:104, doi:10.1016/j.pbi.2014.07.007).

To determine whether RLPs could function in heterologous hosts independently of the presence of their native common adaptor kinase SOBIR1, we evaluated the function of chimeric receptors containing the ectodomain of AtRLP23 and the kinase of AtSOBIR1. Additionally, we also evaluated the function of chimeras containing the ectodomain of AtRLP23 and the kinase domain of other RLKs involved in plant defence to explore whether the function of the AtRLP23 PRR could be improved by other kinase fusions.

32×10⁴ corn protoplasts, that were prepared as described in Example 1, were co-transfected with 10 μg of reporter construct (ZmUbi::R-GECO1.2::rbcS, SEQ ID NO: 75) and 10 μg of pUC19 (SEQ ID NO: 74) or one of the following AtRLP23 constructs: AtRLP23-3×FLAG (SEQ ID NO: 13), AtRLP23-OsXA21-3×FLAG (SEQ ID NO: 21), AtRLP23-AtEFR-3×FLAG (SEQ ID NO: 17), AtRLP23+TM-AtEFR-3×FLAG (SEQ ID NO: 33), AtRLP23-AtBAK1-3×FLAG (SEQ ID NO: 25), AtRLP23+TM-AtBAK1-3×FLAG (SEQ ID NO: 37), AtRLP23-AtSOBIR1-3×FLAG (SEQ ID NO: 29) or AtRLP23+TM-AtSOBIR1-3×FLAG (SEQ ID NO: 41). All of the AtRLP23 constructs were expressed under the control of an operably linked 2×35S+Ω promoter construct (SEQ ID NO: 72) and also contained an operably linked rbcS terminator (SEQ ID NO: 73).

25 μL of protoplasts (8×10⁴ protoplasts) were transferred to each well of a white 384-well microplate (Corning, low volume non-binding surface, model 3824), and then 12.5 μL of protoplast incubation buffer or 3 μM PpNLP20 peptide (AIMYSWYFPKDSPVTGLGHR, SEQ ID NO: 63; prepared by diluting a 100 μM stock solution with protoplast incubation buffer) was added to each well. Fluorescence was monitored using a fluorimeter equipped with a 100 Hz xenon flash lamp with excitation at 556 nm and emission measured at 585 nm for 30 ms, every 22.6 seconds for 40 minutes. For each fluorescence measurement, the transient increase of fluorescence was measured over background. Background was determined, for a given construct, as the average signal observed in response to buffer. Total transient increase of fluorescence was measured for 40 minutes from the time of treatment.

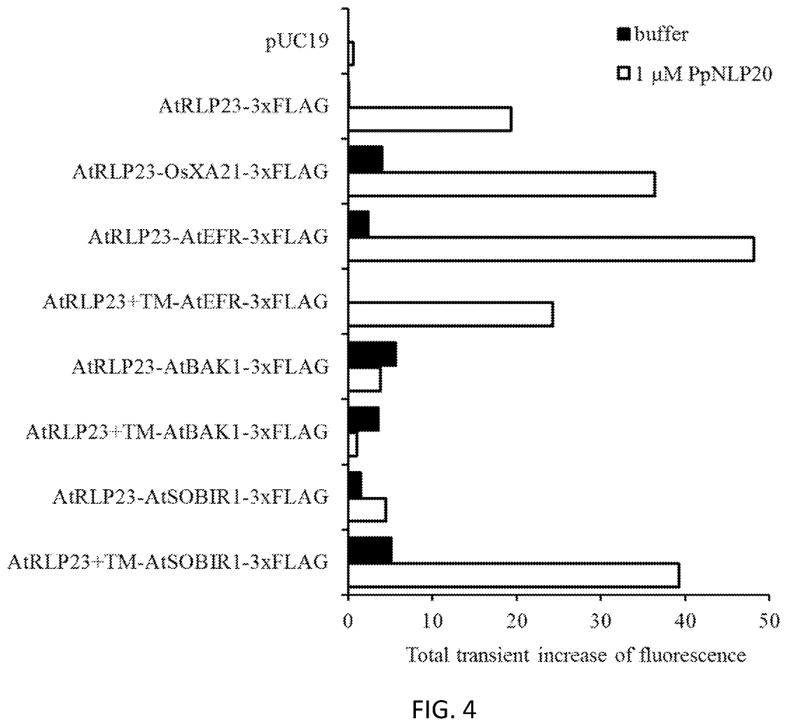

As shown in FIG. 4, transient expression of AtRLP23-AtBAK1-3×FLAG, AtRLP23+TM-AtBAK1-3×FLAG or AtRLP23-AtSOBIR1-3×FLAG in corn protoplasts led to a reduced responsiveness to PpNLP20 treatment compared to AtRLP23-3×FLAG. Transient expression of AtRLP23+TM-AtEFR-3×FLAG or AtRLP23-3×FLAG in corn protoplasts led to comparable responsiveness to PpNLP20 treatment. Transient expression of AtRLP23-OsXA21-3×FLAG, AtRLP23-AtEFR-3×FLAG or AtRLP23+TM-AtSOBIR1-3×FLAG in corn protoplasts led to an increased responsiveness to PpNLP20 treatment when compared to AtRLP23-3×FLAG.

Plant kinases comprise a large multigene family in plants; for example, 940 kinases are present in the Arabidopsis thaliana genome, comprising 3.4% of gene models in this species. The kinase domains of RLKs form a monophyletic group that shares a common origin with animal Pelle and related cytoplasmic kinases (Shiu and Bleeker, 2001. Proc. Natl. Acad. Sci. 98: 10763-10768). In Arabidopsis, 620 sequences related to RLKs were identified and further divided into 46 structural classes defined by their extracellular domains, with the largest group (comprising 14 subgroups) containing leucine-rich repeat (LRR) domains (Shiu and Bleecker, 2001). Some cytoplasmic kinases are also found within the RLK monophyletic grouping. Similar phylogenetic relationships and division into groups and subgroups of RLKs was shown in multiple plant species including monocots and eudicots (reviewed in Lehti-Shiu and Shiu, 2012. Phil. Trans. R. Soc. B. 367: 2619-2639).

Fusion of the AtRLP23 ectodomain with the kinase domains of AtEFR or OsXA21, belonging to LRR-XII subgroup, and the AtSOBIR kinase domain, from a distinct subgroup with a distant phylogenetic relationship to AtEFR and OsXA21 within the RLKs (Dufayard et al., 2017. Front Plant Sci. 2017. doi: 10.3389/fpls.2017.00381; Fischer et al., 2016. Plant Physiol. 170:1595-610) led to an increased responsiveness to PpNLP20 treatment. Therefore, we expect that many kinase domains within the RLK superfamily, such as, for example, any one or more of those presented in SEQ ID NOS: 77-240 and 561-563 would also perform similarly when fused with AtRLP23 ectodomain. To generate a list of kinase domains representing the RLK superfamily, we used 46 Arabidopsis RLKs representatives as defined by Shiu and Bleecker, 2003. Plant. Physiol. 132: 530-543, supplemented with AtSOBIR1 (At2G31880), OsXA21 (Os11g0559200), and with AtFSL2 replaced by AtEFR (At5G20480). Using full-length protein sequences, we performed a blastp search against a manually curated RLK collection from rice, tomato and Medicago. The hit with highest score from each plant species was selected and all the kinase domains were extracted, resulting in 164 kinase domains from these four plant species.

### Example 4: AtRLP23-eJMAtRLP42-AtEFR Function can be Assayed in Protoplasts Using an Aequorin Reporter

Following ligand detection by PRR, the transient increase of cytosolic Ca²⁺ concentration can be monitored in a population of plant protoplasts expressing the genetically encoded proteinaceous bioluminescent Ca²⁺ sensor apoaequorin from the jellyfish Aequorea victoria. The complex formed between apoaequorin and its prosthetic group, the luciferin coelenterazine, is termed aequorin, which upon binding of three Ca²⁺ ions emits light at a wavelength of 470 nm.

Corn protoplasts were isolated from leaves of 10-day old maize seedlings that had been kept in the dark at 25° C. essentially as described in Sheen, 1990. Plant Cell 2:1027, doi:10.1105/tpc.2.10.1027. 32×10⁴ corn (Zea mays) protoplasts were co-transfected with 10 μg of PRR construct (AtRLP23-eJMAtRLP42-AtEFR-3×FLAG, SEQ ID NO: 61) and 10 μg of reporter construct (ZmUbi::Apoaequorin::rbcS, SEQ ID NO: 76) by PEG-mediated transformation using an adaptation of the method of Yoo et al. (2007) Nat Protocols 2:1565-1572. After overnight-resting, transiently transfected corn protoplasts were incubated for 2 hours with 1 μM coelenterazine essentially as described for Arabidopsis protoplasts in Maintz et al., 2014. Plant Cell Physiol. 55:1813, doi:10.1093/pcp/pcu112. 100 μL of transfected protoplasts (32×10⁴ protoplasts) were transferred to each well of a white 96-well microplate (Greiner Bio-One, Lumitrac, model 655075), and then 50 μL of protoplast incubation buffer or 3 uM PpNLP20 peptide (AIMYSWYFPKDSPVTGLGHR, SEQ ID NO: 63; prepared by diluting a 100 μM stock solution with protoplast incubation buffer) was added to each well. Luminescence was monitored immediately and for 60 minutes using a High Resolution Photon Counting System (HRPCS) camera.

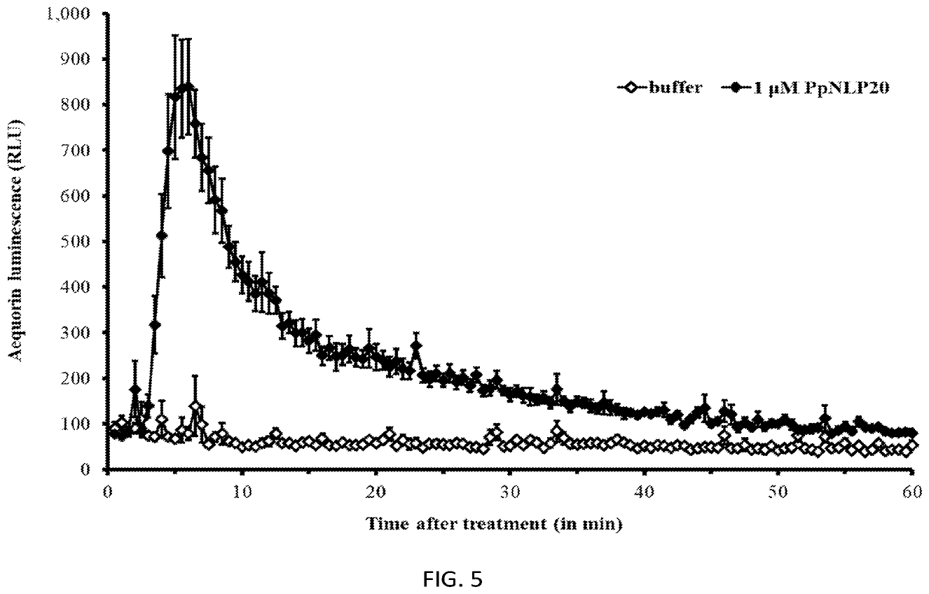

As shown in FIG. 5, transient expression of AtRLP23-eJMAtRLP42-AtEFR-3×FLAG (schematically represented in FIG. 1) in corn protoplasts led to a significant Ca²⁺ increase in response to PpNLP20 treatment but not in response to the protoplast incubation buffer. These results demonstrate that AtRLP23-eJMAtRLP42-AtEFR-3×FLAG expression in corn protoplast led to PpNLP20 perception.

### Example 5: AtRLP23 Also Confers Recognition of NLP20 Peptides from Four Corn Pathogens

Corn stalk and ear rots are caused by Colletotrichum graminicola, Fusarium verticillioides, Fusarium graminearum and Stenocarpella maydis while Aspergillus ear rot is caused by Aspergillus flavus. To determine if the ectopic expression of AtRLP23 in corn could lead to the perception of NLP epitopes present in these fungal pathogen of corn, we have searched their genomes for candidate proteins with homology to the NEP1 protein from Fusarium oxysporum f. sp. erythroxyli. (GenBank: AAC97382.1). We identified several candidate genes (Table 1) and ordered the synthetic peptides corresponding to the orthologous regions of the PpNLP24 epitope (described as 10 times more active than PpNLP20 in Bohm et al., 2014. PLoS Pathog. 10:e1004491, doi:10.1371/journal.ppat.1004491).

TABLE 1
NLP peptides from identified from corn pathogens

| | | |
|---|---|---|
| Gene | Peptide | Sequence |

Phytophthora parasitica

| | |
|---|---|
| PpNLP20 | AIMYSWYFPKDSPVTGLGHR |
| | (SEQ ID NO: 63) |

Colletotrichum graminicola M1.001

| | | |
|---|---|---|
| GLRG_10303 | CgNLP24b | AIMYAYYMPKDSPSPGLGHRHDWE |
| | | (SEQ ID NO: 64) |

Fusarium graminearum PH-1

| | | |
|---|---|---|
| FGSG_06017 | FgNLP24c | AIMYSWYMPKDSPSPGLGHRHDWE |
| | | (SEQ ID NO: 65) |

Fusarium verticillioides 7600

| | | |
|---|---|---|
| FVEG_04647 | FvNLP24a | IMYSWYMPKDSPSPGLGHRHDWE |
| | | (SEQ ID NO: 66) |

Stenocarpella maydis A1-1

| | |
|---|---|
| SmNLP24 | VVMYCWYMPKDQPLDGNTAGGHRHE |
| | FE (SEQ ID NO: 67) |

Aspergillus flavus NRRL3357

| | | |
|---|---|---|
| AFLA_096450 | AfNLP24a | GIMYAWYMPKDMPNSGVSTGAHRHD |
| | | WE (SEQ ID NO: 68) |
| AFLA_054320 | AfNLP24b | ALMYSWYFPKDEPSTGLGHRHDWE |
| | | (SEQ ID NO: 69) |
| AFLA_013750 | AfNLP24c | ALMYSWYFPKDQAAPGMGHRHDWE |
| | | (SEQ ID NO: 70) |

Aspergillus parasiticus SU-1

| | | |
|---|---|---|
| P875_00075739 | ApNLP24a | GIMYAWYMPKDMPNSGVSAGAHRHD |
| | | WE (SEQ ID NO: 71) |

A sample comprising 32×10⁴ corn protoplasts, that were prepared as described in Example 4, were co-transfected with 10 μg of reporter construct ZmUbi::Apoaequorin::rbcS (SEQ ID NO: 76) and 10 μg of either AtRLP23-eJMAtRLP42-3×FLAG (SEQ ID NO: 57) or AtRLP23-eJMAtRLP42-AtEFR-3×FLAG (SEQ ID NO: 61). After incubation as described in Example 4, the protoplasts were treated with 50 μL of either protoplast incubation buffer, 3 μM PpNLP20 peptide (AIMYSWYFPKDSPVTGLGHR, SEQ ID NO: 63), 3 μM CgNLP24b peptide (AIMYAYYMPKDSPSPGLGHRHDWE, SEQ ID NO: 64), 3 μM FgNLP24c peptide (AIMYSWYMPKDSPSPGLGHRHDWE, SEQ ID NO: 65), 3 μM FvNLP24a peptide (IMYSWYMPKDSPSPGLGHRHDWE, SEQ ID NO: 66), or 3 μM SmNLP24 peptide (VVMYCWYMPKDQPLDGNTAGGHRHEFE, SEQ ID NO: 67). Luminescence was assayed as described above.

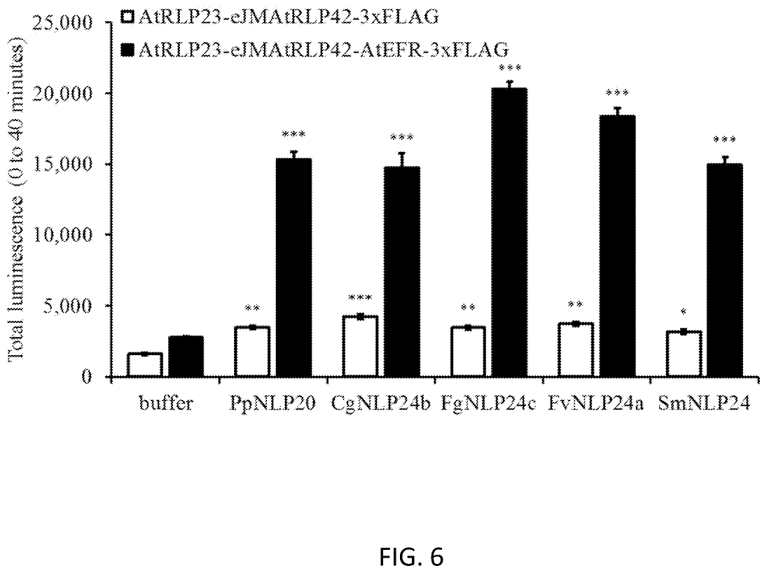

As shown in FIG. 6, transient expression of AtRLP23-eJMAtRLP42-3×FLAG (SEQ ID NO: 57) or AtRLP23-eJMAtRLP42-AtEFR-3×FLAG (SEQ ID NO: 61) in corn protoplasts led to a Ca²⁺ increase in response to PpNLP20 (from Phytophthora parasitica), CgNLP24b (from Colletotrichum graminicola M1.001), FgNLP24c (from Fusarium graminearum PH-1), FvNLP24a (from Fusarium verticillioides 7600) and SmNLP24 (from Stenocarpella maydis A1-1). For these five peptides, a stronger response was observed in the presence of AtRLP23-eJMAtRLP42-AtEFR-3×FLAG than in the presence of AtRLP23-eJMAtRLP42-3×FLAG.

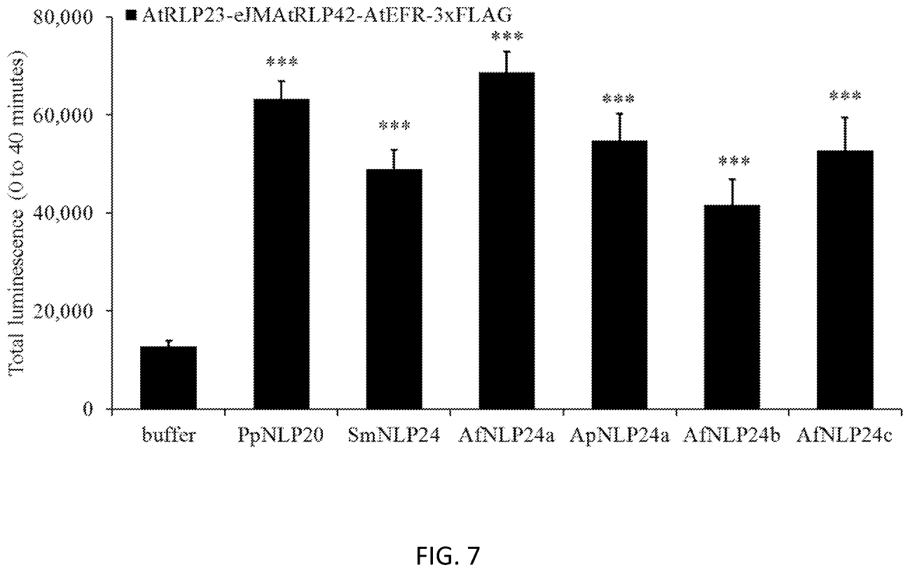

The response of AtRLP23-eJMAtRLP42-AtEFR-3×FLAG (SEQ ID NO: 61) to further NLPs from corn pathogens was then assayed as described above. As shown in FIG. 7, transient expression of AtRLP23-eJMAtRLP42-AtEFR-3×FLAG (SEQ ID NO: 61) in corn protoplasts led to a Ca²⁺ increase in response to PpNLP20 (from Phytophthora parasitica), SmNLP24 (from Stenocarpella maydis A1-1), AfNLP24a (from Aspergillus flavus NRRL3357), ApNLP24a (from Aspergillus parasiticus SU-1), AfNLP24b (from Aspergillus flavus NRRL3357 and Aspergillus parasiticus SU-1) and AfNLP24c (from Aspergillus flavus NRRL3357 and Aspergillus parasiticus SU-1).

### Example 6: Fusions of the AtRLP23 Ectodomain with Kinase Domains of RLK Superfamily Members

To test if fusion of other kinases within the RLK superfamily (described above in Example 3) with the AtRLP23 ectodomain leads to recognition of at least one of NLP sequences, we generated chimeric constructs fusing AtRLP23 ectodomain (SEQ ID NO: 560) and eJM with TM and kinase domains from OsCERK1 (SEQ ID NOS: 331, 433, and 239, respectively), Os01g49614 (SEQ ID NOS: 369, 518, and 122, respectively), OsPi-d2 (SEQ ID NOS: 564, 567, and 561, respectively), Medtr3g3g095100 (SEQ ID NOS: 310, 514, and 224, respectively), Mt7g073660 (SEQ ID NOS: 279, 438, and 172, respectively), AtWAK1 (SEQ ID NOS: 364, 512, and 107, respectively), AtPEPR1 (SEQ ID NOS: 565, 568, and 562, respectively), AtLYK5 (SEQ ID NOS: 566, 568, and 563, respectively), AtExtensin (SEQ ID NOS: 273, 527, and 133, respectively), AtC_lectin (SEQ ID NOS: 350, 506, and 128, respectively), Solyc01g108000 (SEQ ID NOS: 280, 499, and 233, respectively), or Solyc06g051030 (SEQ ID NOS: 253, 427, and 221, respectively).

32×10⁴ corn protoplasts samples prepared as described in Example 4, were co-transfected with 10 μg of reporter construct ZmUbi::Apoaequorin::rbcS (SEQ ID NO: 76) and 10 μg of each one of the chimeras described above. After incubation as described in Example 4, the protoplasts were treated with 50 μL of either protoplast incubation buffer, or 3 μM CgNLP24b peptide (SEQ ID NO: 64), 3 μM FgNLP24c peptide (SEQ ID NO: 65), 3 μM FvNLP24a peptide (SEQ ID NO: 66) or 3 μM SmNLP24 peptide (SEQ ID NO: 67). Luminescence was assayed as described above.

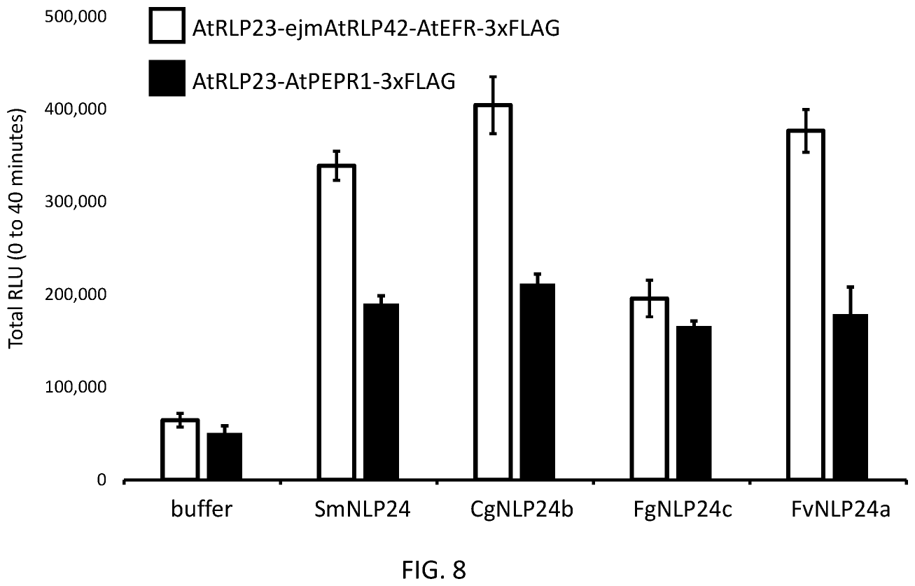

As shown in FIG. 8, only transient expression of AtRLP23-AtPEPR1-3×FLAG chimera (SEQ ID NO: 572) in corn protoplasts led to a Ca²⁺ burst in response to all tested NLP peptides (data not shown for the other chimeras). The response with the AtRLP23-AtPEPR1-3×FLAG chimera was similar (FgNLP24c) or weaker (all other NLPs) in strength to the response to control construct AtRLP23-ejmAtRLP42-AtEFR-3×FLAG. However, the increase in Ca²⁺ for AtRLP23-AtPEPR1-3×FLAG chimera was statistically significant compared to control buffer treatment for all peptides tested.

### Example 7: Transgenic Corn Plants Expressing Modified AtRLP23 Genes have Enhanced Resistance to Diplodia Stalk Rot

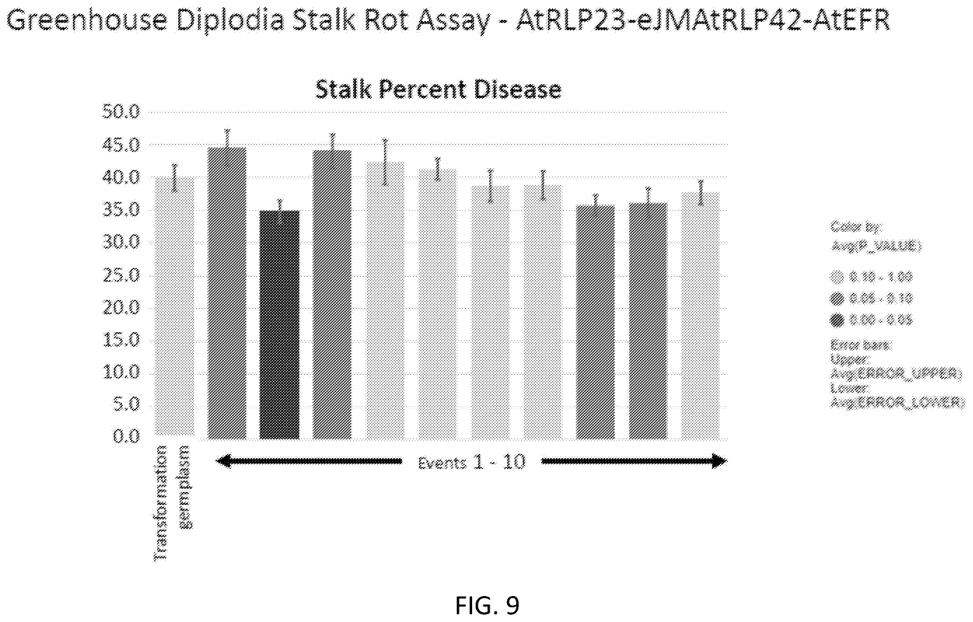

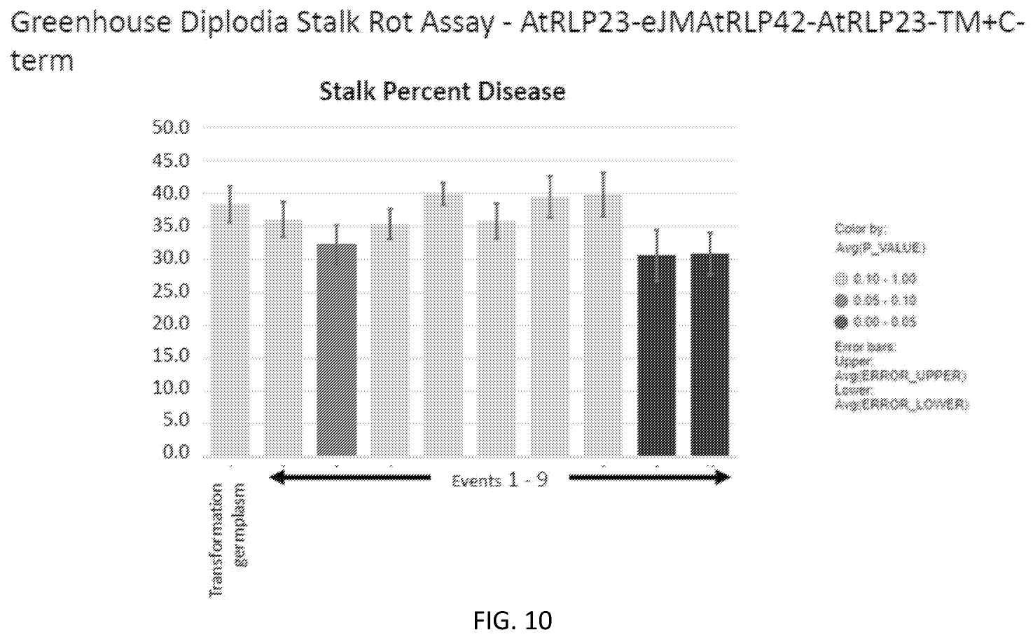

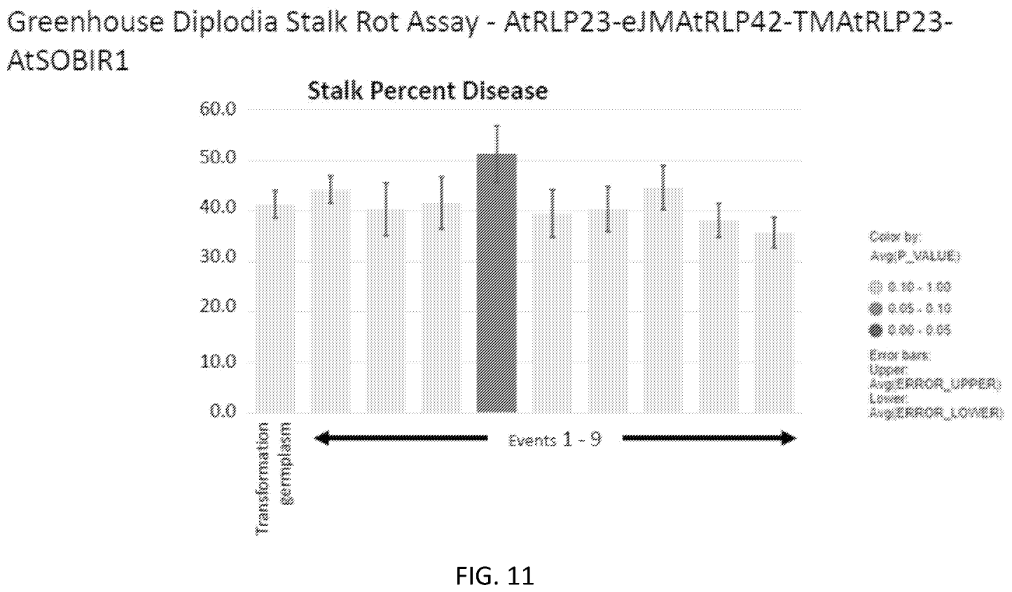

Transgenic corn (Zea mays) plants at the T1 stage expressing modified AtRLP23 genes under the control of a constitutive promoter were generated and grown in the greenhouse. At the VT growth stage, two nodes were infected by wounding the stalk and injecting a suspension of Diplodia maydis pathogen inoculum into the wound. Sixteen plants from each event were inoculated. Corn plants were evaluated for Diplodia stalk rot approximately 2-3 weeks after inoculation. Stalks were harvested, leaves were removed, and the stalks were split longitudinally. Disease severity was reported as the percent necrosis of the cut surface and is compared to the percent necrosis of non-transformed plants as shown in FIGS. 9-11. Transgenic corn plants expressing modified AtRLP23 genes have enhanced disease resistance to Diplodia maydis.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. An isolated nucleic acid molecule encoding an engineered AtRLP23 protein, the nucleic acid molecule comprising a nucleotide sequence encoding a polypeptide which comprises in operable linkage: a leucine-rich-repeat (LRR) domain derived from AtRLP23, an extra-juxtamembrane (eJM) domain, a transmembrane (TM) domain, and optionally a kinase domain derived from a receptor-like kinase (RLK).

2. The nucleic acid molecule of claim 1, wherein the engineered AtRLP23 protein is capable of recognizing in a plant a pathogen-associated molecular pattern derived from a Nep1-like protein (NLP).

3. The nucleic acid molecule of claim 1, wherein:
(a) the kinase domain is a kinase domain derived from OsXA21, AtSOBIR1, AtPEPR1, or AtEFR;
(b) the eJM domain is an eJM domain derived from at least one of AtRLP1, AtRLP23, AtRLP30, AtRLP42, Cf-4, and Ve1;
(c) the eJM domain is eJM(EEEE/ADQ−); and/or
(d) the polypeptide further comprises a signal peptide (SP) domain operably linked to the LRR domain.

4-6. (canceled)

7. The nucleic acid molecule of claim 3, wherein at least one of the SP domain and the TM domain is derived from a plasmalemma-bound protein and optionally, wherein the plasmalemma-bound protein is a pattern recognition receptor (PRR).

8. (canceled)

9. The nucleic acid molecule of claim 7, wherein the PRR is selected from the group consisting of AtRLP1, AtRLP23, AtRLP30, AtRLP42, Cf-4, Ve1, AtSOBIR1, and AtEFR.

10-12. (canceled)

13. An engineered AtRLP23 protein encoded by the encoded by the nucleic acid molecule of claim 1.

14. An expression cassette comprising the nucleic acid molecule of claim 1 and an operably linked promoter for expression in a host cell of interest or a vector comprising the nucleic acid molecule or the expression cassette.

15-16. (canceled)

17. A plant, plant cell, or other host cell comprising the nucleic acid molecule of claim 1.

18. A plant or plant cell comprising stably incorporated in its genome a polynucleotide construct comprising a nucleotide sequence encoding an engineered AtRLP23 protein, wherein the nucleotide sequence encodes a polypeptide comprising in operable linkage: a leucine-rich-repeat (LRR) domain derived from AtRLP23, an extra-juxtamembrane (eJM) domain, a transmembrane (TM) domain, and a kinase domain derived from a receptor-like kinase (RLK).

19. The plant or plant cell of claim 18, wherein the polypeptide further comprises a signal peptide (SP) domain operably linked to the LRR domain.

20. The plant or plant cell of claim 19, wherein at least one of the SP domain and the TM domain is derived from a plasmalemma-bound protein.

21. The plant or plant cell of claim 20, wherein the plasmalemma-bound protein is a pattern recognition receptor (PRR).

22-24. (canceled)

25. The plant or plant cell of claim 18, wherein the engineered AtRLP23 protein is capable of recognizing in a plant or plant cell a pathogen-associated molecular pattern derived from a Nep1-like protein (NLP).

26. The plant or plant cell of claim 18, wherein the polynucleotide construct further comprises a promoter operably linked for the expression of the nucleotide sequence in the plant or plant cell.

27. The plant or plant cell of any one of claim 18, wherein the plant or a plant regenerated from the plant cell comprises enhanced resistance to at least one plant disease caused by a plant pathogen, relative to the resistance of a control plant lacking the polynucleotide construct.

28. (canceled)

29. The plant or plant cell of claim 27, wherein the plant pathogen comprises an NLP.

30. A method for making an engineered AtRLP23 protein, the method comprising producing a polypeptide comprising in operable linkage: a leucine-rich-repeat (LRR) domain derived from AtRLP23, an extra-juxtamembrane (eJM) domain, a transmembrane (TM) domain, and a kinase domain derived from a receptor-like kinase (RLK).

31-39. (canceled)

40. An isolated, engineered AtRLP23 protein produced by the method of claim 30, an isolated nucleic acid molecule encoding the engineered AtRLP23 protein, or a plant or plant cell comprising the engineered AtRLP23 protein and/or the nucleic acid molecule.

41. A method for making a nucleic acid molecule encoding an engineered AtRLP23 protein, the method comprising synthesizing a nucleic acid molecule which encodes a polypeptide comprising in operable linkage: a leucine-rich-repeat (LRR) domain derived from AtRLP23, an extra-juxtamembrane (eJM) domain, a transmembrane (TM) domain, and a kinase domain derived from a receptor-like kinase (RLK).

42-50. (canceled)

51. An isolated nucleic acid molecule produced by the method of claim 41, an isolated, engineered AtRLP23 protein encoded by the nucleic acid molecule, or a plant or plant cell comprising the nucleic acid molecule and/or the engineered AtRLP23 protein.

52. (canceled)

53. A method for enhancing the resistance of a plant to at least one plant pathogen, the method comprising modifying a plant cell to be capable of expressing
the engineered AtRLP23 protein encoded by the nucleic acid molecule of claim 1.

54-58. (canceled)

59. A plant or plant cell produced by the method of claim 53.
